SEQUENCE LISTING (82 sequences)

SEQ ID NO: 1 - protein, 11 aa, synthetic construct
RASQSVSSYLA

SEQ ID NO: 2 - protein, 7 aa, synthetic construct
DASNRAT

SEQ ID NO: 3 - protein, 9 aa, synthetic construct
QQRSNWPPT

SEQ ID NO: 4 - protein, 7 aa, synthetic construct
NARMGVS

SEQ ID NO: 5 - protein, 16 aa, synthetic construct
HIFSNDDKSYSPSLKS

SEQ ID NO: 6 - protein, 10 aa, synthetic construct
MRYSGGPFDY

SEQ ID NO: 7 - protein, 5 aa, synthetic construct
RSGMH

SEQ ID NO: 8 - protein, 17 aa, synthetic construct
LISFDGSNKYYADSVKG

SEQ ID NO: 9 - protein, 13 aa, synthetic construct
GGFGVVIYDAFDI

SEQ ID NO: 10 - protein, 7 aa, synthetic construct
SNNYYWA

SEQ ID NO: 11 - protein, 16 aa, synthetic construct
SLYYTGRAYYKSSLKS

SEQ ID NO: 12 - protein, 13 aa, synthetic construct
HDCGNTCLNWFDR

SEQ ID NO: 13 - protein, 7 aa, synthetic construct
NIRMGVS

SEQ ID NO: 14 - protein, 16 aa, synthetic construct
HIFSNDDKSYSPSLKN

SEQ ID NO: 15 - protein, 10 aa, synthetic construct
MKYSGGPFDY

SEQ ID NO: 16 - protein, 7 aa, synthetic construct
NIRMGVS

SEQ ID NO: 17 - protein, 16 aa, synthetic construct
HIFSNDDKSYSTSLKS

SEQ ID NO: 18 - protein, 10 aa, synthetic construct
MRYSGGPFDY

SEQ ID NO: 19 - protein, 11 aa, synthetic construct
RASQSVSSYLA

SEQ ID NO: 20 - protein, 7 aa, synthetic construct
DASNRAT

SEQ ID NO: 21 - protein, 9 aa, synthetic construct
QQRSNWPPT

SEQ ID NO: 22 - protein, 9 aa, synthetic construct
TFSLSNARM

SEQ ID NO: 23 - protein, 5 aa, synthetic construct
FSNDD

SEQ ID NO: 24 - protein, 10 aa, synthetic construct
MRYSGGPFDY

SEQ ID NO: 25 - protein, 7 aa, synthetic construct
GFTFSRS

SEQ ID NO: 26 - protein, 6 aa, synthetic construct
SFDGSN

SEQ ID NO: 27 - protein, 13 aa, synthetic construct
GGFGVVIYDAFDI

SEQ ID NO: 28 - protein, 9 aa, synthetic construct
GGSISSNNY

SEQ ID NO: 29 - protein, 5 aa, synthetic construct
YYTGR

SEQ ID NO: 30 - protein, 13 aa, synthetic construct
HDCGNTCLNWFDR

SEQ ID NO: 31 - protein, 9 aa, synthetic construct
GFSLSNIRM

SEQ ID NO: 32 - protein, 5 aa, synthetic construct
FSNDD

SEQ ID NO: 33 - protein, 10 aa, synthetic construct
MKYSGGPFDY

SEQ ID NO: 34 - protein, 9 aa, synthetic construct
GFSLSNIRM

SEQ ID NO: 35 - protein, 5 aa, synthetic construct
FSNDD

SEQ ID NO: 36 - protein, 10 aa, synthetic construct
MRYSGGPFDY

SEQ ID NO: 37 - protein, 107 aa, synthetic construct [sequence omitted]

SEQ ID NO: 38 - protein, 120 aa, synthetic construct
QVQLVQSGPVLVKPTETLTLTCTVSTFSLSNARMGVSWIRQPPGKALEWLAHIFSNDDKSYSPSLKSRLTISKDTSKTQVVLTMTNMDPVDTATYYCSRMRYSGGPFDYWGQGSLVTVSS

SEQ ID NO: 39 - protein, 122 aa, synthetic construct
QVQLVESGGGVVQPGKSLRLSCAASGFTFSRSGMHWVRQAPGKGLEWVALISFDGSNKYYADSVKGRFTISRDNSKNTLNLQMNSLRAEDTAVYYCAKGGFGVVIYDAFDIWGHGTMVTVSS

SEQ ID NO: 40 - protein, 123 aa, synthetic construct
EVQLVQSGPGLVKPSETLSLTCTVSGGSISSNNYYWAWIRQPPGKGLEWIGSLYYTGRAYYKSSLKSRVTISVDTSNDQFSLRLTSVTAADTAIYYCARHDCGNTCLNWFDRWGQGTLVTVSS

SEQ ID NO: 41 - protein, 120 aa, synthetic construct
EVQLVQSGPVLAKPTETLTLTCTVSGFSLSNIRMGVSWIRQPPGKALEWLAHIFSNDDKSYSPSLKNRLTISKDTSKSQVVLTMTTMDPMDTATYYCTRMKYSGGPFDYWGQGTLVTVSS

SEQ ID NO: 42 - protein, 120 aa, synthetic construct
QVQLVQSGPVLVKPTETLTLTCTVSGFSLSNIRMGVSWIRQPPGKALEWLAHIFSNDDKSYSTSLKSRLTISKDTSKSQVVLTMTNVDPVDTATYYCTRMRYSGGPFDYWGQGTLVTVSS

SEQ ID NO: 43 - protein, 624 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLT

SEQ ID NO: 44 - protein, 624 aa, Macaca mulatta
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWKDIVRDQDAEIVVKDNGRSCPLCHEVCKGRCWGPGPEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMYNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLT

SEQ ID NO: 45 - protein, 1338 aa, Canis lupus
MRATAPLQVLGFLLSLVRASYVGNSQAVCPGTLNGLSVTGDAENQYQTLYKLYERCEVVMGNLEIVLTGHNADLSFLQWIREVTGYVLVAMNEFPTLPLPNLRVVRGTQVYDGKFAIFVMLNYNTNSSHALRQLRFTQLTEILAGGVYIEKNDKLCHMDTIDWRDIVRDRDAEIVVKDNGRSCPPCHETCKGRCWGPRPEDCQTLTKTICAPQCNGHCFGPNPNQCCHDECAGGCSGPQDTDCFACRLFNDSGACVRQCPQPLVYNKLTFQLEPNPHTKYQYGGVCVASCPHNFVVDQTSCVRACPPQMEVDKNGSKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNITSLGLRSLKEISAGRIYISANKQLCYHHSLNWTRLLRGPPEERLDIKHNRPRRDCVAEGKVCDPLCSSGGCWGPGPGQCLSCRNYSRGGVCVTHCNFLNGEPREFAHEAECFSCHPECQPMEGTATCNGSGSDACAQCAHFRDGPHCVSSCPNGVLGAKGPIYKYPDTHNECRPCHENCTQGCKGPELQDCLGQTLALISKTHLAVGLTVVVGLAVIFLILGGTLLYWRGRRIQNKRAMRRYLERGESIEPLDPSEKANKVLARIFKETELRKLKVLGSGVFGTVHKGVWIPEGESIKIPVCIKVIEDKSGRQSFQDVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLHSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIPPGAEPPALTNKELEEVELEPELELDLDLETEEDGLAATLNSALGLPVGTLNRPRGSQSLLSPSSGYMPMNQGNLGDTCQESAICGTGERCPRPASLHPMPRGRLASESSEGHVTGSEAELQEKASMCRSRSRSPRPRGDSAYHSQRHSLLTPVTPLSPPGLEEEDVNGYVMPDAHLKGTPSSREGTLSSVGISSVLGTEEEEEDEEYEYMNRRRRHSPPRHPRPSSLEELGYEYMDVGSDLSASLGSTQSCPLNPVPLMPAAGTTPDEDYEYMNRRHAGGAPGGDYAAMGACPAAEQGYEEMRAFQGPGNHAPHVHCARLKPLRSLEATDSAFDNPDYWHSRLFPKADAQRT

SEQ ID NO: 46 - protein, 622 aa, Mus musculus
SEMGNSQAVCPGTLNGLSVTGDADNQYQTLYKLYEKCEVVMGNLEIVLTGHNADLSFLQWIREVTGYVLVAMNEFSVLPLPNLRVVRGTQVYDGKFAIFVMLNYNTNSSHALRQLRFTQLTEILLGGVYIEKNDKLCHMDTIDWRDIVRVPDAEIVVKNNGGNCPPCHEVCKGRCWGPGPEDCQILTKTICAPQCNGRCFGPNPNQCCHDECAGGCSGPQDTDCFACRHFNDSGACVPRCPAPLVYNKLTFQLEPNPHIKYQYGGVCVASCPHNFVVDQTFCVRACPADKMEVDKNGLKMCEPCRGLCPKACEGTGSGSRYQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRVYISANQQLCYHHSLNWTRLLRGPAEERLDIKYNRPLGECVAEGKVCDPLCSSGGCWGPGPGQCLSCRNYSREGVCVTHCNVLQGEPREFVHEAHCFSCHPECQPMEGTSTCNGSGSDACARCAHFRDGPHCVNSCPHGILGAKGPIYKYPDAQNECRPCHENCTQGCKGPELQDCLGQAEVLMSKPH

SEQ ID NO: 47 - protein, 117 aa, synthetic construct [sequence omitted]

SEQ ID NO: 48 - protein, 113 aa, synthetic construct [sequence omitted]

SEQ ID NO: 49 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 5 aa, synthetic construct
SNYWS

SEQ ID NO: 52 - protein, 16 aa, synthetic construct
YIYHSGSTNYNPSLKS

SEQ ID NO: 53 - protein, 14 aa, synthetic construct
GPVSIFGVVKYIDL

SEQ ID NO: 54 - protein, 7 aa, synthetic construct
TSGVAVA

SEQ ID NO: 55 - protein, 16 aa, synthetic construct
LIYWDDDKWYTPSLKT

SEQ ID NO: 56 - protein, 13 aa, synthetic construct
RGSFNWNYGYFDY

SEQ ID NO: 57 - protein, 7 aa, synthetic construct
GGSISSN

SEQ ID NO: 58 - protein, 5 aa, synthetic construct
YHSGS

SEQ ID NO: 59 - protein, 14 aa, synthetic construct
GPVSIFGVVKYIDL

SEQ ID NO: 60 - protein, 9 aa, synthetic construct
GFSLYTSGV

SEQ ID NO: 61 - protein, 5 aa, synthetic construct
YWDDD

SEQ ID NO: 62 - protein, 13 aa, synthetic construct
RGSFNWNYGYFDY

SEQ ID NO: 63 - protein, 122 aa, synthetic construct
EVQLVESGPGLVKPSETLSLTCTVSGGSISSNYWSWIRQPPGKGLEWIGYIYHSGSTNYNPSLKSRVTISVDTSKNQFSLKLSSVTAADTAVYFCARGPVSIFGVVKYIDLWGRGTLVTVSS

SEQ ID NO: 64 - protein, 123 aa, synthetic construct
QVQLVQSGPALVKPTQTLTLTCTFSGFSLYTSGVAVAWIRQPPGKALEWLALIYWDDDKWYTPSLKTRLSITKDTSKNQVVLTMTNMDPVDTATYYCAHRGSFNWNYGYFDYWGQGTLVTVSS

SEQ ID NO: 65 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 66 - protein, 1400 aa, Macaca mulatta
MKSKSKGLAECLAHDKPLTMKAPAVLVPGILVLLFTLVQRSNGECKEALAKSEMNVNMKYQLPNFTAETAIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPNQCPDCVVSALGAKVLSSVKDRFINFFVGNTINSSYFPHHPLHSISVRRLKETKDGFMFLTDQSYIDVLPEFRDSYPIKYIHAFESNNFIYFLTVQRETLNAQTFHTRIIRFCSLNSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISIALLLLLGLFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDT

SEQ ID NO: 67 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 68 - protein, 111 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 69 - protein, 5 aa, synthetic construct
GHYWS

SEQ ID NO: 70 - protein, 16 aa, synthetic construct
YITYSGSTNYNPSLKS

SEQ ID NO: 71 - protein, 14 aa, synthetic construct
IAVAGRVYYYGVDV

SEQ ID NO: 72 - protein, 5 aa, synthetic construct
GYYWT

SEQ ID NO: 73 - protein, 16 aa, synthetic construct
ESNHGGSTNFNPSLKS

SEQ ID NO: 74 - protein, 13 aa, synthetic construct
GGTIFGLKDAFDI

SEQ ID NO: 75 - protein, 7 aa, synthetic construct
GGSISGH

SEQ ID NO: 76 - protein, 5 aa, synthetic construct
TYSGS

SEQ ID NO: 77 - protein, 14 aa, synthetic construct
IAVAGRVYYYGVDV

SEQ ID NO: 78 - protein, 7 aa, synthetic construct
GGSFSGY

SEQ ID NO: 79 - protein, 5 aa, synthetic construct
NHGGS

SEQ ID NO: 80 - protein, 13 aa, synthetic construct
GGTIFGLKDAFDI

SEQ ID NO: 81 - protein, 122 aa, synthetic construct
QVQLQESGPGLVKPSETLSLTCTVSGGSISGHYWSWIRQSPGKGLEWIGYITYSGSTNYNPSLKSRVTISVDTSKNQFSLKLSSVTAADTAVYYCGTIAVAGRVYYYGVDVWGRGTTVTVSS

SEQ ID NO: 82 - protein, 121 aa, synthetic construct
QVQLQQWGAGLLKPSETLSLTCAVYGGSFSGYYWTWIRQPPGKGLEWIGESNHGGSTNFNPSLKSRVTISVDTSKNHFSLKLSPVTAADTAVYYCTRGGTIFGLKDAFDIWGQGTMVTVSS

(19) United States
(12) Patent Application Publication
(10) Pub. No.: US 2026/0250418 A1
(43) Pub. Date: Aug. 27, 2026

(54) ANTI-HER3 AND ANTI-MET ANTIBODIES AND USES THEREOF

(71) Applicant: Biocytogen Pharmaceuticals (Beijing) Co., Ltd., Beijing (CN)

(72) Inventors: Zhuolin Li, Beijing (CN); Chengzhang Shang, Beijing (CN); Yi Yang, Beijing (CN); Yuelei Shen, Beijing (CN)

(21) Appl. No.: 19/160,296

(22) PCT Filed: Mar. 15, 2024
(86) PCT No.: PCT/CN2024/081833

§ 371 Date: Aug. 27, 2025

(30) Foreign Application Priority Data
Mar. 17, 2023 (WO) PCT/CN2023/082295
Mar. 24, 2023 (WO) PCT/CN2023/083790
Oct. 19, 2023 (WO) PCT/CN2023/125481
Feb. 2, 2024 (WO) PCT/CN2024/075481

(51) Int. Cl. C07K 16/32; A61K 38/00; A61K 39/395; A61K 47/68; A61P 35/00; C07K 14/705; C07K 16/28

(52) U.S. Cl. CPC C07K 16/32; A61K 39/3955; A61K 47/68031; A61K 47/68037; A61K 47/6849; A61K 47/6889; A61P 35/00; C07K 14/7051; C07K 16/2818; C07K 16/2827; C07K 16/2863; C07K 16/2878; A61K 38/00; C07K 2317/31; C07K 2317/622

(57) ABSTRACT

Provided are anti-HER3 (human epidermal growth factor receptor 3) and anti-MET antibodies, and the related anti-gen-binding fragments thereof, antibody-drug conjugate (ADC) derived therefrom, and the uses thereof.

### CLAIM OF PRIORITY

This application claims priority to PCT/CN2023/082295, filed on Mar. 17, 2023; PCT/CN2023/083790, filed on Mar. 24, 2023; PCT/CN2023/125481, filed on Oct. 19, 2023; and PCT/CN2024/075481, filed on Feb. 2, 2024. The entire contents of the foregoing applications are incorporated herein by reference.

### TECHNICAL FIELD

This disclosure relates to antibodies, antigen-binding fragments thereof, antibody-drug conjugate (ADC) derived therefrom, and the uses thereof.

### BACKGROUND

ERBB family members are aberrantly activated in multiple cancers and hence serve as drug targets and biomarkers in modern precision therapy. HER3 has received attention in recent years as it is a crucial heterodimeric partner for other EGFR family members and has the potential to regulate EGFR/HER2-mediated resistance. Upregulation of HER3 is associated with several malignancies where it fosters tumor progression via interaction with different receptor tyrosine kinases (RTKs).

MET, also called tyrosine-protein kinase MET or hepatocyte growth factor receptor (HGFR) is a protein that in humans is encoded by the MET gene. MET pathway plays an important role in the development of cancer through: activation of key oncogenic pathways (RAS, PI3K, STAT3, beta-catenin), angiogenesis (sprouting of new blood vessels from pre-existing ones to supply a tumor with nutrients), and scatter (cells dissociation due to metalloprotease production).

Considering the important role of HER3 and MET in tumors, there is a need to develop a therapeutic agent targeting HER3 and/or MET.

### SUMMARY

This disclosure relates to anti-HER3 antibodies, antigen-binding fragment thereof, and the uses thereof.

In one aspect, the disclosure is related to an antibody or antigen-binding fragment thereof that binds to HER3 (human epidermal growth factor receptor 3) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 19-21, respectively;
(7) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 19-21, respectively;
(8) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 19-21, respectively;
(9) the selected VH CDRs 1, 2, 3 amino acid sequences are set forth in SEQ ID NOs: 31-33, respectively, and the selected VL CDRs 1, 2, 3 amino acid sequences are set forth in SEQ ID NOs: 19-21, respectively; and
(10) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 19-21, respectively.

In some embodiments, the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 19-21, respectively, according to Chothia definition.

In some embodiments, the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 19-21, respectively, according to Chothia definition.

In some embodiments, the VH comprises CDRs 1, 2, 3 with the amino acid sequences set forth in SEQ ID NOs: 28-30, respectively, and the VL comprises CDRs 1, 2, 3 with the amino acid sequences set forth in SEQ ID NOs: 19-21, respectively, according to Chothia definition.

In some embodiments, the VH comprises CDRs 1, 2, 3 with the amino acid sequences set forth in SEQ ID NOs: 31-33, respectively, and the VL comprises CDRs 1, 2, 3 with the amino acid sequences set forth in SEQ ID NOs: 19-21, respectively, according to Chothia definition.

In some embodiments, the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 19-21, respectively, according to Chothia definition.

In some embodiments, the antibody or antigen-binding fragment thereof specifically binds to human, mouse, monkey, or dog HER3.

In some embodiments, the antibody or antigen-binding fragment thereof is a human antibody or antigen-binding fragment thereof, a single-chain variable fragment (scFv), a one-armed antibody, and/or a multi-specific antibody (e.g., a bispecific antibody).

In some embodiments, the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

In one aspect, the disclosure is related to an antibody or antigen-binding fragment thereof that binds to HER3 comprising a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In some embodiments, the VH comprises the sequence of SEQ ID NO: 38 and the VL comprises the sequence of SEQ ID NO: 37.

In some embodiments, the VH comprises the sequence of SEQ ID NO: 39 and the VL comprises the sequence of SEQ ID NO: 37.

In some embodiments, the VH comprises the sequence of SEQ ID NO: 40 and the VL comprises the sequence of SEQ ID NO: 37.

In some embodiments, the VH comprises the sequence of SEQ ID NO: 41 and the VL comprises the sequence of SEQ ID NO: 37.

In some embodiments, the VH comprises the sequence of SEQ ID NO: 42 and the VL comprises the sequence of SEQ ID NO: 37.

In one aspect, the disclosure is related to an antibody or antigen-binding fragment thereof that binds to HER3 comprising a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37. In some embodiments, the antibody or antigen-binding fragment thereof specifically binds to human, mouse, monkey, or dog HER3.

In some embodiments, the antibody or antigen-binding fragment thereof is a human antibody or antigen-binding fragment thereof, a single-chain variable fragment (scFv), a one-armed antibody, and/or a multi-specific antibody (e.g., a bispecific antibody).

In some embodiments, the antibody or antigen-binding fragment is a human IgG1 antibody or antigen-binding fragment thereof, a human IgG2 antibody or antigen-binding fragment thereof, or a human IgG4 antibody or antigen-binding fragment thereof.

In one aspect, the disclosure is related to an antibody or antigen-binding fragment thereof, comprising: a first antigen-binding domain that specifically binds to a first epitope of HER3; and a second antigen-binding domain that specifically binds to a second epitope of HER3.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively;
(7) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 19-21, respectively;
(8) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 19-21, respectively;
(9) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively; and
(10) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 4-6, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 10-12, respectively, and the selected VL2 CDRs 1, 2, 3 amino acid sequences are set forth in SEQ ID NOs: 1-3, respectively;
(4) the selected VH2 CDRs 1, 2, 3 amino acid sequences are set forth in SEQ ID NOs: 13-15, respectively, and the selected VL2 CDRs 1, 2, 3 amino acid sequences are set forth in SEQ ID NOs: 1-3, respectively;
(5) the selected VH2 CDRs 1, 2, 3 amino acid sequences are set forth in SEQ ID NOs: 16-18, respectively, and the selected VL2 CDRs 1, 2, 3 amino acid sequences are set forth in SEQ ID NOs: 1-3, respectively;
(6) the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 19-21, respectively;
(7) the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively;
(8) the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 19-21, respectively;
(9) the selected VH2 CDRs 1, 2, 3 amino acid sequences are set forth in SEQ ID NOs: 31-33, respectively, and the selected VL2 CDRs 1, 2, 3 amino acid sequences are set forth in SEQ ID NOs: 19-21, respectively; and
(10) the selected VH2 CDRs 1, 2, 3 amino acid sequences are set forth in SEQ ID NOs: 34-36, respectively, and the selected VL2 CDRs 1, 2, 3 amino acid sequences are set forth in SEQ ID NOs: 19-21, respectively.

In some embodiments, the selected VH2 CDRs 1, 2, 3 amino acid sequences are set forth in SEQ ID NOs: 4-6, respectively, and the selected VL2 CDRs 1, 2, 3 amino acid sequences are set forth in SEQ ID NOs: 1-3, respectively.

In some embodiments, the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively.

In some embodiments, the selected VH2 CDRs 1, 2, 3 amino acid sequences are set forth in SEQ ID NOs: 10-12, respectively, and the selected VL2 CDRs 1, 2, 3 amino acid sequences are set forth in SEQ ID NOs: 1-3, respectively.

In some embodiments, the selected VH2 CDRs 1, 2, 3 amino acid sequences are set forth in SEQ ID NOs: 13-15, respectively, and the selected VL2 CDRs 1, 2, 3 amino acid sequences are set forth in SEQ ID NOs: 1-3, respectively.

In some embodiments, the selected VH2 CDRs 1, 2, 3 amino acid sequences are set forth in SEQ ID NOs: 16-18, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively.

In some embodiments, the VH1 comprises an amino acid sequence that is at least 80%, 85%, 90%, 95%, 99%, or 100% identical to a selected VH sequence, and the VL1 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In some embodiments, the VH2 comprises an amino acid sequence that is at least 80%, 85%, 90%, 95%, 99%, or 100% identical to a selected VH sequence, and the VL2 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In one aspect, the disclosure is related to an antibody or antigen-binding fragment thereof, comprising: a first antigen-binding domain that specifically binds to HER3; and a second antigen-binding domain that specially binds to MET.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively;
(7) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 19-21, respectively;
(8) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 19-21, respectively;
(9) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively; and
(10) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 19-21, respectively;
(4) the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 19-21, respectively;
(5) the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(6) the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively;
(7) the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 19-21, respectively; and
(8) the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively.

In some embodiments, the VH1 comprises an amino acid sequence that is at least 90% identical to a selected VH sequence, and the VL1 comprises an amino acid sequence that is at least 90% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In some embodiments, the VH2 comprises an amino acid sequence that is at least 90% identical to a selected VH sequence, and the VL2 comprises an amino acid sequence that is at least 90% identical to a selected VL sequence, wherein the selected VH sequence and the selected VL sequence are one of the following:
(1) the selected VH sequence is SEQ ID NO: 63, and the selected VL sequence is SEQ ID NO: 37;
(2) the selected VH sequence is SEQ ID NO: 64, and the selected VL sequence is SEQ ID NO: 37;
(3) the selected VH sequence is SEQ ID NO: 81, and the selected VL sequence is SEQ ID NO: 37; and
(4) the selected VH sequence is SEQ ID NO: 82, and the selected VL sequence is SEQ ID NO: 37.

In one aspect, the disclosure is related to an antibody or antigen-binding fragment thereof that cross-competes with the antibody or antigen-binding fragment thereof described herein.

In some embodiments, the antibody or antigen-binding fragment thereof comprises a fragment crystallizable region (Fc region).

In some embodiments, the Fc region has increased complement-dependent cytotoxicity (CDC) or antibody-dependent cellular cytotoxicity (ADCC).

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 4-6, respectively, and wherein the VH, when paired with a light chain variable region (VL) comprising the amino acid sequence set forth in SEQ ID NO: 37 binds to HER3;
(2) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 38 binds to HER3;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7-9, respectively, and wherein the VH, when paired with a light chain variable region (VL) comprising the amino acid sequence set forth in SEQ ID NO: 37 binds to HER3;
(4) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 39 binds to HER3;
(5) an immunoglobulin heavy chain or a fragment thereof comprising a VH comprising complementarity determining regions (CDRs) 1, 2, and 3 comprising the amino acid sequences set forth in SEQ ID NOs: 10-12, respectively, and wherein the VH, when paired with a light chain variable region (VL) comprising the amino acid sequence set forth in SEQ ID NO: 37 binds to HER3;
(6) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 40 binds to HER3;
(7) an immunoglobulin heavy chain or a fragment thereof comprising a VH comprising complementarity determining regions (CDRs) 1, 2, and 3 comprising the amino acid sequences set forth in SEQ ID NOs: 13-15, respectively, and wherein the VH, when paired with a light chain variable region (VL) comprising the amino acid sequence set forth in SEQ ID NO: 37 binds to HER3;
(8) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 41 binds to HER3;
(9) an immunoglobulin heavy chain or a fragment thereof comprising a VH comprising complementarity determining regions (CDRs) 1, 2, and 3 comprising the amino acid sequences set forth in SEQ ID NOs: 16-18, respectively, and wherein the VH, when paired with a light chain variable region (VL) comprising the amino acid sequence set forth in SEQ ID NO: 37 binds to HER3;
(10) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 42 binds to HER3;
(11) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 22-24, respectively, and wherein the VH, when paired with a light chain variable region (VL) comprising the amino acid sequence set forth in SEQ ID NO: 37 binds to HER3;
(12) an immunoglobulin light chain or a fragment thereof comprising a VL comprising complementarity determining regions (CDRs) 1, 2, and 3 comprising the amino acid sequences set forth in SEQ ID NOs: 19-21, respectively, and wherein the VL, when paired with a VH comprising the amino acid sequence set forth in SEQ ID NO: 38 binds to HER3;
(13) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25-27, respectively, and wherein the VH, when paired with a light chain variable region (VL) comprising the amino acid sequence set forth in SEQ ID NO: 37 binds to HER3;
(14) an immunoglobulin light chain or a fragment thereof comprising a VL comprising complementarity determining regions (CDRs) 1, 2, and 3 comprising the amino acid sequences set forth in SEQ ID NOs: 19-21, respectively, and wherein the VL, when paired with a VH comprising the amino acid sequence set forth in SEQ ID NO: 39 binds to HER3;
(15) an immunoglobulin heavy chain or a fragment thereof comprising a VH comprising complementarity determining regions (CDRs) 1, 2, and 3 comprising the amino acid sequences set forth in SEQ ID NOs: 28-30, respectively, and wherein the VH, when paired with a light chain variable region (VL) comprising the amino acid sequence set forth in SEQ ID NO: 37 binds to HER3;
(16) an immunoglobulin light chain or a fragment thereof comprising a VL comprising CDRs 1, 2, and 3 comprising the amino acid sequences set forth in SEQ ID NOs: 19-21, respectively, and wherein the VL, when paired with a VH comprising the amino acid sequence set forth in SEQ ID NO: 40 binds to HER3;
(17) an immunoglobulin heavy chain or a fragment thereof comprising a VH comprising complementarity determining regions (CDRs) 1, 2, and 3 comprising the amino acid sequences set forth in SEQ ID NOs: 31-33, respectively, and wherein the VH, when paired with a light chain variable region (VL) comprising the amino acid sequence set forth in SEQ ID NO: 37 binds to HER3;
(18) an immunoglobulin light chain or a fragment thereof comprising a VL comprising CDRs 1, 2, and 3 comprising the amino acid sequences set forth in SEQ ID NOs: 19-21, respectively, and wherein the VL, when paired with a VH comprising the amino acid sequence set forth in SEQ ID NO: 41 binds to HER3;
(19) an immunoglobulin heavy chain or a fragment thereof comprising a VH comprising complementarity determining regions (CDRs) 1, 2, and 3 comprising the amino acid sequences set forth in SEQ ID NOs: 34-36, respectively, and wherein the VH, when paired with a light chain variable region (VL) comprising the amino acid sequence set forth in SEQ ID NO: 37 binds to HER3; or
(20) an immunoglobulin light chain or a fragment thereof comprising a VL comprising CDRs 1, 2, and 3 comprising the amino acid sequences set forth in SEQ ID NOs: 19-21, respectively, and wherein the VL, when paired with a VH comprising the amino acid sequence set forth in SEQ ID NO: 42 binds to HER3.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1-3, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 19-21, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 4-6, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7-9, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 10-12, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13-15, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 16-18, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 22-24, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 25-27, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 28-30, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 31-33, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 34-36, respectively.

In some embodiments, the VH when paired with a VL specifically binds to human, mouse, monkey, or dog HER3, or the VL when paired with a VH specifically binds to human, mouse, monkey, or dog HER3.

In some embodiments, the immunoglobulin heavy chain or the fragment thereof is a human immunoglobulin heavy chain or a fragment thereof (e.g., a human IgG1 heavy chain or a fragment thereof, a human IgG2 antibody or antigen-binding fragment thereof, or a human IgG4 heavy chain or a fragment thereof), and the immunoglobulin light chain or the fragment thereof is a human immunoglobulin light chain or a fragment thereof.

In some embodiments, the nucleic acid encodes a single-chain variable fragment (scFv), a one-armed antibody, a multi-specific antibody (e.g., a bispecific antibody), or a chimeric antigen receptor (CAR).

In some embodiments, the nucleic acid is cDNA.

In one aspect, the disclosure is related to a nucleic acid encodes the antibody or antigen-binding fragment thereof described herein.

In one aspect, the disclosure is related to a vector comprising one or more of the nucleic acids described herein.

In one aspect, the disclosure is related to a vector comprising two of the nucleic acids described herein, wherein the vector encodes the VL region and the VH region that together bind to HER3.

In one aspect, the disclosure is related to a pair of vectors, wherein each vector comprises one of the nucleic acids described herein, wherein together the pair of vectors encodes the VL region and the VH region that together bind to HER3.

In one aspect, the disclosure is related to a cell comprising the vector described herein, or the pair of vectors described herein.

In some embodiments, the cell is a CHO cell.

In one aspect, the disclosure is related to a cell comprising one or more of the nucleic acids described herein.

In one aspect, the disclosure is related to a method of producing an antibody or an antigen-binding fragment thereof, the method comprising
(a) culturing the cell described herein under conditions sufficient for the cell to produce the antibody or the antigen-binding fragment; and
(b) collecting the antibody or the antigen-binding fragment produced by the cell.

In one aspect, the disclosure is related to a chimeric antigen receptor (CAR) comprising the antibody or antigen-binding fragment thereof described herein.

In one aspect, the disclosure is related to an antibody-drug conjugate comprising the antibody or antigen-binding fragment thereof described herein covalently bound to a therapeutic agent.

In some embodiments, the therapeutic agent is a cytotoxic or cytostatic agent.

In some embodiments, the therapeutic agent is MMAE or MMAF.

In some embodiments, the therapeutic agent is selected from

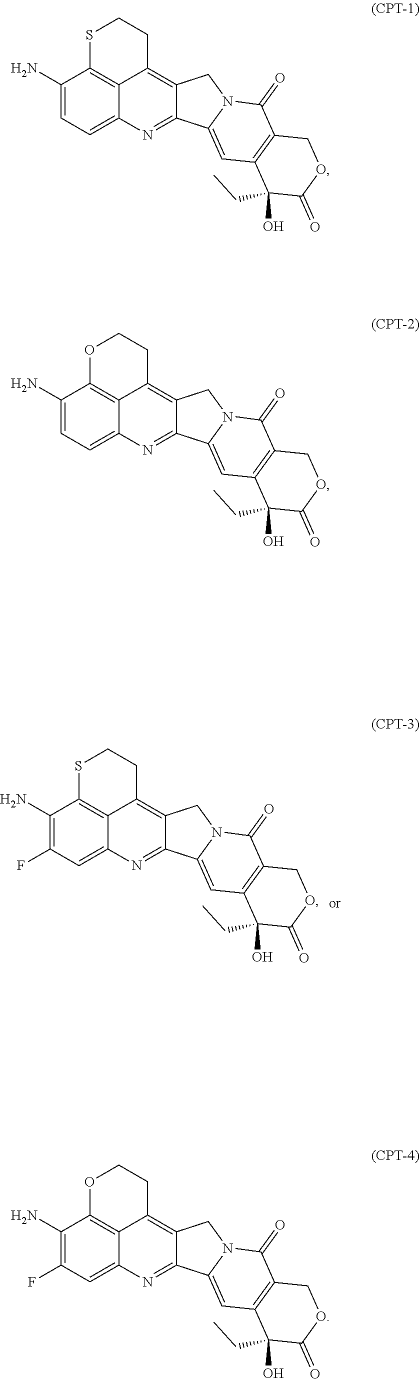 [SMILES: CC[C@@]1(O)C(=O)OCc2c1cc1n(c2=O)Cc2c-1nc1ccc(N)c3c1c2CCS3 ; CC[C@@]1(O)C(=O)OCc2c1cc1n(c2=O)Cc2c-1nc1ccc(N)c3c1c2CCO3 ; CC[C@@]1(O)C(=O)OCc2c1cc1n(c2=O)Cc2c-1nc1cc(F)c(N)c3c1c2CCS3 ; CC[C@@]1(O)C(=O)OCc2c1cc1n(c2=O)Cc2c-1nc1cc(F)c(N)c3c1c2CCO3]

In some embodiments, the therapeutic agent is linked to the antibody or antigen-binding fragment thereof via a linker. In some embodiments, the linker has a structure of:

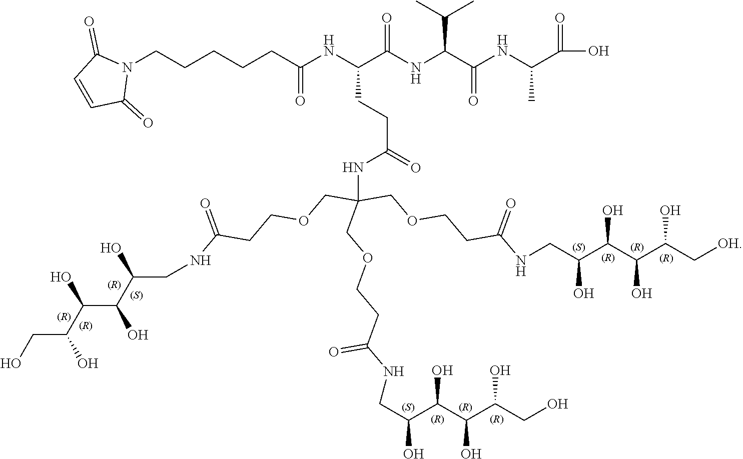

In some embodiments, the antibody-drug conjugate has a structure of:

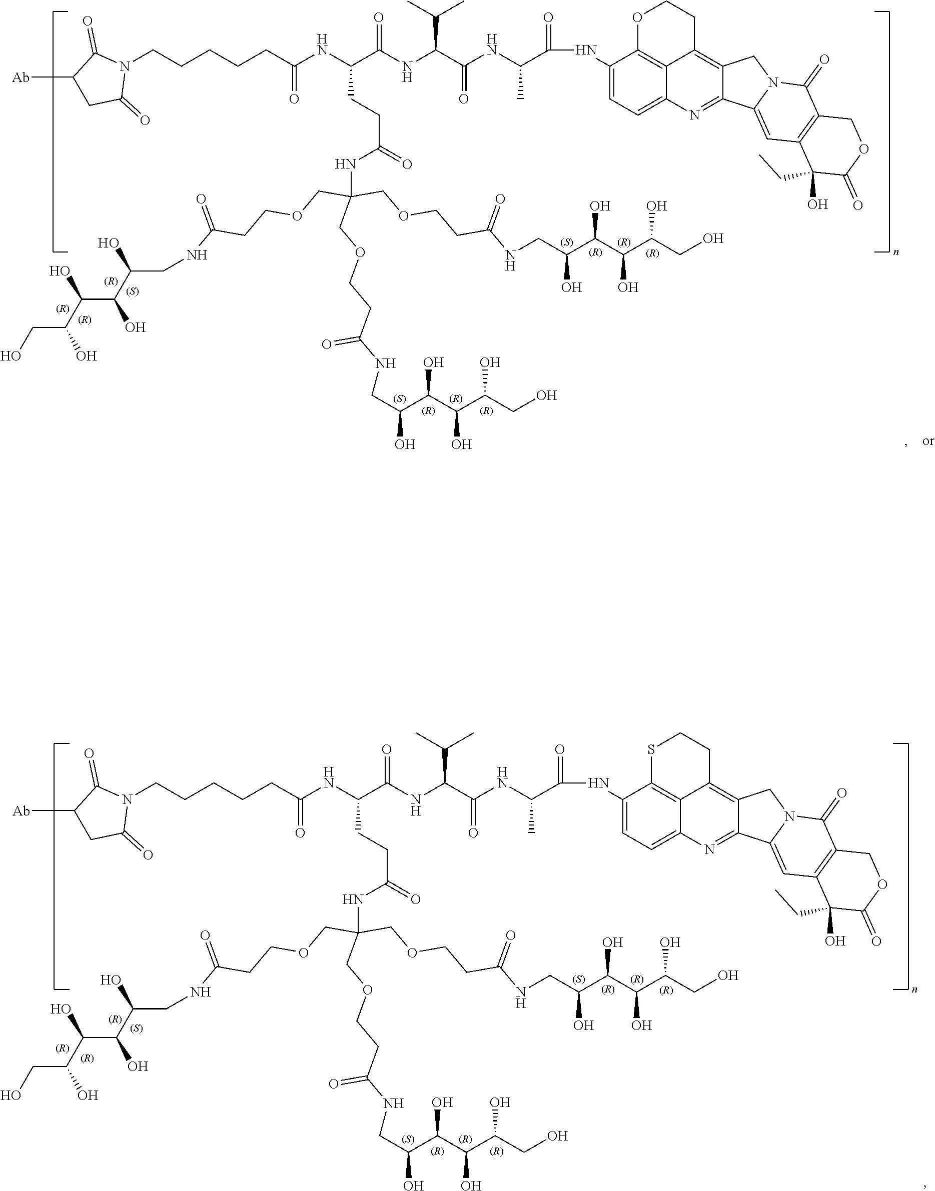

in some embodiments, n=1, 2, 3, 4, 5, 6, 7, or 8; in some embodiments, “Ab” represents the antibody or antigen-binding fragment thereof described herein.

In one aspect, the disclosure is related to a method of treating a subject having cancer, the method comprising administering a therapeutically effective amount of a composition comprising the antibody or antigen-binding fragment thereof described herein, the CAR described herein, or the antibody-drug conjugate described herein, to the subject.

In some embodiments, the subject has a solid tumor.

In some embodiments, the cancer is colorectal cancer, gastric cancer, breast cancer, lung cancer, melanoma, ovarian cancer, head and neck cancer, pancreatic cancer or cervical cancer.

In some embodiments, the subject is further treated with an effective amount of an anti-4-1BB antibody, an anti-OX40 antibody, an anti-PD-1 antibody, an anti-CTLA4 antibody, an anti-CD40 antibody, or an anti-PD-L1 antibody.

In one aspect, the disclosure is related to a method of decreasing the rate of tumor growth, the method comprising contacting a tumor cell with an effective amount of a composition comprising an antibody or antigen-binding fragment thereof described herein, the CAR described herein, or the antibody-drug conjugate described herein.

In one aspect, the disclosure is related to a method of killing a tumor cell, the method comprising contacting a tumor cell with an effective amount of a composition comprising the antibody or antigen-binding fragment thereof described herein, the CAR described herein, or the antibody-drug conjugate described herein.

In one aspect, the disclosure is related to a method of increasing immune response in a subject, the method comprising administering to the subject an effective amount of a composition comprising the antibody or antigen-binding fragment thereof described herein, the CAR described herein, or the antibody-drug conjugate described herein.

In one aspect, the disclosure is related to a pharmaceutical composition comprising the antibody or antigen-binding fragment thereof described herein, and a pharmaceutically acceptable carrier.

In one aspect, the disclosure is related to a pharmaceutical composition comprising the antibody drug conjugate described herein, and a pharmaceutically acceptable carrier.

In some embodiments, the drug-to-antibody ratio (DAR) is about 4 or 8.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody.

Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the term “multispecific antibody” is an antibody that includes two or more different antigen-binding domains that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, a multispecific antibody binds two different epitopes (i.e., a “bispecific antibody”). In some aspects, a multispecific antibody binds three different epitopes (i.e., a “trispecific antibody”). In some aspects, a multispecific antibody binds four different epitopes (i.e., a “quadspecific antibody”). In some aspects, a multispecific antibody binds five different epitopes (i.e., a “quintspecific antibody”). Each binding specificity may be present in any suitable valency. Non-limiting examples of multispecific antibodies are described herein.

As used herein, the term “bispecific antibody” refers to an antibody that binds to two different epitopes. The epitopes can be on the same antigen or on different antigens.

As used herein, the term “common light chain” refers to a light chain that can interact with two or more different heavy chains, forming different antigen-binding sites, wherein these different antigen-binding sites can specifically bind to different antigens or epitopes. Similarly, the term “common light chain variable region” refers to a light chain variable region that can interact with two or more different heavy chain variable regions, forming different antigen-binding sites, wherein these different antigen-binding sites can specifically bind to different antigens or epitopes. In some embodiments, the antibody or antigen-binding fragment thereof can have a common light chain. In some embodiments, the antibody or antigen-binding fragment thereof as described herein can have a common light chain variable region.

As used herein, the term “anti-HER3/MET antibody or antigen-binding fragment thereof” refers to an antibody or antigen-binding fragment that binds to both MET and HER3.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., HER3) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a HER3 molecule may be referred to as a HER3-specific antibody or an anti-HER3 antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1G show cross binding activities of anti-HER3 antibodies to HER family proteins as determined by surface plasmon resonance (SPR).

FIG. 2 shows the average tumor volume in different groups of B-NDG mice that were subcutaneously injected with stomach cancer NUGC-4 cells, and were treated with PBS or ADCs.

FIG. 3A lists heavy chain and light chain variable region CDR sequences of anti-HER3 antibodies as defined by Kabat numbering.

FIG. 3B lists heavy chain and light chain variable region CDR sequences of anti-MET antibodies as defined by Kabat numbering.

FIG. 4A lists heavy chain and light chain variable region CDR sequences of anti-HER3 antibodies as defined by Chothia numbering.

FIG. 4B lists heavy chain and light chain variable region CDR sequences of anti-MET antibodies as defined by Chothia numbering.

FIG. 5 lists heavy chain and light chain variable regions of anti-HER3 antibodies and anti-MET antibodies.

FIG. 6 shows the average tumor volume in different groups of B-NDG mice that were subcutaneously injected with stomach cancer NUGC-4 cells, and were treated with PBS, ISO-ADC or ADCs.

FIG. 7 shows the average tumor volume in different groups of B-NDG mice that were subcutaneously injected with stomach cancer NUGC-4 cells, and were treated with PBS or bispecific ADCs.

FIG. 8 shows the average tumor volume in different groups of B-NDG mice that were subcutaneously injected with pancreatic cancer fragments, and were treated with PBS or ADCs.

FIG. 9 shows exemplary bispecific antibodies with antigen binding arms derived from different antibodies.

FIG. 10 lists selected amino acid sequences discussed in the disclosure.

FIG. 11 shows the average tumor volume in different groups of B-NDG mice that were subcutaneously injected with pancreatic cancer fragments, and were treated with PBS or ADCs.

FIGS. 12A-12C show the in vitro killing activities of ADCs in NUGC-4 cells, SNU-5 cells and PLC-PRF-5 cells.

FIG. 13 shows the average tumor volume in different groups of B-NDG mice that were subcutaneously injected with gastric cancer fragments, and were treated with PBS or ADCs.

FIG. 14 shows the average tumor volume in different groups of B-NDG mice that were subcutaneously injected with pancreatic cancer fragments, and were treated with PBS or ADCs.

FIG. 15 shows the average tumor volume in different groups of B-NDG mice that were subcutaneously injected with colorectal cancer cells, and were treated with PBS or ADCs.

FIG. 16 shows the average tumor volume in different groups of B-NDG mice that were subcutaneously injected with gastric cancer fragments, and were treated with PBS or ADCs.

FIG. 17 shows the average tumor volume in different groups of B-NDG mice that were subcutaneously injected with colorectal cancer fragments, and were treated with PBS or ADCs.

FIG. 18 shows the average tumor volume in different groups of B-NDG mice that were subcutaneously injected with colorectal cancer fragments, and were treated with PBS or ADCs.

### DETAILED DESCRIPTION

The present disclosure provides examples of antibodies, antigen-binding fragment thereof, that bind to HER3 and/or MET.

HER3 is a pseudokinase member of the EGFR family having a role in both tumor progression and drug resistance. HER3 is an exceptional EGFR family member that is not oncogenic alone, but can cooperate with other receptors to induce tumorigenesis, metastatic events, and drug resistance. HER3 is a compelling cancer therapeutic target. In contrast to EGFR and HER2, which have been broadly targeted with TKIs, HER3 has been mainly targeted with monoclonal or bispecific antibodies due to the minimal kinase activity, either by blocking the ligand binding or heterodimerization with other receptors.

The HER proteins are a family of receptor tyrosine kinases that play a role in both normal and tumor cell biology. The family consists of four highly homologous members, EGFR (ERBB1/HER1), HER2 (ERBB2), HER3 (ERBB3), and HER4 (ERBB4), consisting of a ligand-binding extracellular domain, a transmembrane domain, an intracellular kinase domain, and a C-terminal tail. The family members (except HER2) are generally activated through extracellular ligand binding, inducing a conformational change, followed by homo- or heterodimerization among the family members, eventually leading to the activation of an intracellular signaling cascade. The cellular responses include increased cell survival and proliferation, explaining why aberrant EGFR family signaling is strongly connected with oncogenic events.

HER3 is a unique EGFR family member with no or little intracellular tyrosine kinase activity. Compared with the other EGFR family members, HER3 diverges at critical residues in the kinase domain, locking it in an inactive-like conformation. Although HER3 has been reported to have some kinase activity, it is suggested to be 1,000-fold weaker than the kinase activity of the fully activated EGFR. Because HER3 is unable to form homodimers, its activation depends on heterodimerization with another receptor to induce the downstream C-terminal phosphorylation events.

The HER3 gene localizes in the long arm of chromosome 12 (12q13.2), encoding a 180 kDa protein. The extracellular domain of HER3 is divided into four subdomains (I-IV): subdomains I and III are leucine-rich β-helical areas responsible for the ligand binding, whereas subdomains II and IV are cysteine-rich regions. Subdomain II also contains a dimerization arm necessary for the interaction with other receptors. The transmembrane domain is followed by an intracellular domain enclosing a flexible juxta membrane region, kinase domain, and the C-terminal tail. In absence of a ligand, binding between subdomains II and IV keeps HER3 in an inactive state. Upon ligand binding, the dimerization partner's kinase domain transphosphorylates the tyrosine residues in the C-terminal tail of HER3.

The preferable dimerization partners for HER3 are EGFR and HER2, followed by lower affinity to HER4. HER3 also dimerizes with some non-EGFR family receptors, including mesenchymal-epithelial transition (MET) factor receptor and FGFR2. Six of 11 HER3 tyrosine phosphorylation sites are direct recruiters of PI3K, making HER3 a strong activator for PI3K/protein kinase B (AKT) signaling, important for cancer cell survival. HER3 also activates MAPK signaling, stimulating cell proliferation. Other suggested effectors of HER3 include JAKs and activators of transcription and proto-oncogene tyrosine-protein kinase SRC signaling pathways involved in signal transduction and increased cell proliferation.

In contrast to other EGFR family members, HER3 is not oncogenic when overexpressed alone. However, ubiquitous HER3 expression is detected in various cancers, including breast, ovarian, colon, gastric, lung, cutaneous, and pancreatic cancers. High HER3 expression is also linked to disease progression and/or poor prognosis in many cancer types.

Although HER3 does not cause tumorigenesis on its own, HER2:HER3 heterodimers possess the highest transforming capability among all the possible EGFR family dimers. The superior oncogenic capability of the dimer pair makes HER3 critical for HER2-mediated tumorigenesis in multiple tumor types. HER3 overexpression is a frequent event, especially in HER2-positive breast cancers, and mice expressing neu (rodent HER2) transgene exhibit elevated HER3 expression. In breast cancer cell lines, HER3 was shown to be critical for maintaining cell viability, while EGFR was dispensable. In addition, inhibition of HER3 revokes HER2-dependent tumorigenesis in transgenic mammary tumor models. HER3 has also been implicated in the pathogenesis of non-small cell lung cancer (NSCLC), many of which have EGFR mutations.

HER3 expression acts as a bypass mechanism for various targeted therapies, and elevated HER3 signaling confers resistance to multiple therapeutic agents. Because HER3 dimerizes with receptors other than EGFR, including HER2 and MET receptor, HER3 can confer resistance to EGFR-targeting therapies via dimerization with partners other than EGFR. Early on, it was shown that HER2:HER3-mediated signaling associates with EGFR tyrosine kinase inhibitor (TKI), gefitinib, resistance in head and neck cancer and in breast cancer. Later, it was shown that HER3 ligand, NRG1, and the following increase in HER2:HER3 dimerization confer resistance to EGFR-directed antibody, cetuximab, in colorectal cancer. Similarly, an increase in EGFR:HER3 dimerization was found in the majority of the residual cancer burden of patients with cetuximab/panitumumab-resistant breast cancer. Other anti-HER TKIs, such as osimertinib, may also induce HER3 upregulation as part of the resistance mechanism. This NRG1-driven EGFR inhibitor resistance was reverted by using HER3-selective antibody, patritumab.

HER3 expression is also connected to resistance to hormonal therapies. HER3 plays a critical role in the phosphorylation of HER2 in breast cancer cells, and downregulation of HER3 reversed antiestrogen receptor (ER) tamoxifen resistance in breast cancer cell lines. Furthermore, patients with breast cancer bearing tumors coexpressing HER2 and HER3 are more prone to develop tamoxifen resistance measured by disease-free survival. Increased activity of EGFR, HER2, and HER3 was also connected to resistance to ER agonist, fulvestrant. Fulvestrant treatment enhanced the HER3 expression and phosphorylation in breast cancer cells in an NRG1-dependent manner, and this was suggested as a resistance mechanism for fulvestrant in breast cancer. In patients with triple-negative breast cancer (TNBC), high HER3/EGFR protein expression (but not HER3 or EGFR alone) conferred worse 10-year survival after chemotherapy. Interestingly, high HER3/EGFR was associated with worse survival following adjuvant chemotherapy, when compared with patients who did not receive adjuvant chemotherapy.

A detailed review of HER3 and its functions can be found in Haikala, Heidi M., and Pasi A. Jänne. “Thirty Years of HER3: From Basic Biology to Therapeutic Interventions 30 Years of HER3.” Clinical Cancer Research 27.13 (2021): 3528-3539; and Mishra, Rosalin, et al. “HER3 signaling and targeted therapy in cancer.” Oncology reviews 12.1 (2018); Liu, Xiaolong, et al. “Development of effective therapeutics targeting HER3 for cancer treatment.” Biological procedures online 21.1 (2019): 1-10; each of which is incorporated by reference in its entirety.

MET, also called c-MET, tyrosine-protein kinase MET, or hepatocyte growth factor receptor (HGFR), is a protein that in humans is encoded by the MET gene. The protein possesses tyrosine kinase activity. The primary single chain precursor protein is post-translationally cleaved to produce the alpha and beta subunits, which are disulfide linked to form the mature receptor. Activation of MET by its ligand hepatocyte growth factor (HGF) stimulates a plethora of cell processes including growth, motility, invasion, metastasis, epithelial-rnesenchynial transition, angiogenesis/wound healing, and tissue regeneration. The exact stoichiometry of HGF:MET binding is unclear, but it is generally believed that two HGF molecules bind to two MET molecules leading to receptor dimerization and autophosphorylation at tyrosines 1230, 1234, and 1235. Ligand-independent MET autophospliorylation can also occur due to gene amplification, mutation or receptor over-expression.

MET is frequently amplified, mutated or over-expressed in many types of cancer including gastric, lung, colon, breast, bladder, head and neck, ovarian, prostate, thyroid, pancreatic, and CNS cancers. Missense mutations typically localized to the kinase domain are commonly found in hereditary papillary renal cell carcinomas (PRCC) and in 13% of sporadic PRCCs, MET mutations localized to the semaphorin or juxtamembrane domains of MET are frequently found in gastric, head and neck, liver, ovarian, NSCLC and thyroid cancers. MET amplification has been detected in brain, colorectal, gastric, and lung cancers, often correlating with disease progression. Up to 4% and 20% of non-small cell lung cancer (NSCLC) and gastric cancers, respectively, exhibit MET amplification. MET overexpression is also frequently observed in lung cancer. Moreover, in clinical samples, nearly half of lung adenocarcinomas exhibited high levels of MET and HGF, both of which correlated with enhanced tumor growth rate, metastasis and poor prognosis.

Nearly 60% of all tumors that become resistant to EGF tyrosine kinase inhibitors increase MET expression, amplify MET, or increase MET only known ligand, HGF, suggesting the existence of a compensatory pathway for EGFR through MET. MET amplification was first identified in cultured cells that became resistant to gefitinib, an EGFR kinase inhibitor, and exhibited enhanced survival through the HER3 pathway. This was further validated in clinical samples where nine of 43 patients with acquired resistance to either erlotinib or gefitinib exhibited MET amplification.

Aberrant MET signaling has been implicated in the development/progression of many human cancers. This results from the overexpression of MET, activating mutations in MET, transactivation, autocrine or paracrine signaling, or MET gene amplification. Binding of HGF to MET stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division, inhibition of MET signaling may result in inhibition of one or more MET downstream signaling pathways and therefore neutralizing MET may have various effects, including inhibition of cell proliferation and differentiation, angiogenesis, cell motility and metastasis.

A detailed review of MET and its functions can be found in Huang, X., et al. “Targeting the HGF/MET axis in cancer therapy: challenges in resistance and opportunities for improvement.” Frontiers in Cell and Developmental Biology 8 (2020): 152; and Santarpia, M., et al. “A narrative review of MET inhibitors in non-small cell lung cancer with MET exon 14 skipping mutations.” Translational Lung Cancer Research 10.3 (2021): 1536; each of which is incorporated by reference in its entirety.

The present disclosure provides several anti-HER3 antibodies, antigen-binding fragments thereof, and methods of using these anti-HER3 antibodies and antigen-binding fragments to inhibit tumor growth, treat cancers, and to treat autoimmune diseases. The present disclosure also provides several anti-MET antibodies, antigen-binding fragments thereof, and methods of using these anti-MET antibodies and antigen-binding fragments to inhibit tumor growth, treat cancers, and to treat autoimmune diseases.

### Anti-HER3 Antibodies and Antigen-Binding Fragments

The disclosure provides antibodies and antigen-binding fragments thereof that specifically bind to HER3 (e.g., human HER3). The antibodies and antigen-binding fragments described herein are capable of binding to HER3. These antibodies can be agonists or antagonists. In some embodiments, these antibodies can increase immune response. In some embodiments, these antibodies can block HER3 activity, e.g., the activity of HER3 heterodimer, such as HER2:HER3 heterodimer.

The disclosure provides e.g., anti-HER3 antibodies 1B2, 1C5, 1D6, 3E1, 3G6, the chimeric antibodies thereof, and the human or humanized antibodies thereof.

The CDR sequences for 1B2, and 1B2 derived antibodies (e.g., human or humanized antibodies) include CDRs of the heavy chain variable domain, SEQ ID NOs: 4-6, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 22-24 and CDR sequences of the light chain variable domain are set forth in SEQ ID NOs: 19-21.

Similarly, the CDR sequences for 1C5, and 1C5 derived antibodies include CDRs of the heavy chain variable domain, SEQ ID NOs: 7-9, and CDRs of the light chain variable domain, SEQ ID NOs: 1-3, as defined by Kabat numbering. Under Chothia numbering, the CDR sequences of the heavy chain variable domain are set forth in SEQ ID NOs: 25-27, and CDRs of the light chain variable domain are set forth in SEQ ID NOs: 19-21.

The CDR sequences for 1D6, and 1D6 derived antibodies include CDRs of the heavy chain variable domain, SEQ ID NOs: 10-12, and CDRs of the light chain variable domain, SEQ ID NOs: 1-3, as defined by Kabat numbering. Under Chothia numbering, the CDR sequences of the heavy chain variable domain are set forth in SEQ ID NOs: 28-30, and CDRs of the light chain variable domain are set forth in SEQ ID NOs: 19-21.

The CDR sequences for 3E1, and 3E1 derived antibodies include CDRs of the heavy chain variable domain, SEQ ID NOs: 13-15, and CDRs of the light chain variable domain, SEQ ID NOs: 1-3, as defined by Kabat numbering. Under Chothia numbering, the CDR sequences of the heavy chain variable domain are set forth in SEQ ID NOs: 31-33, and CDRs of the light chain variable domain are set forth in SEQ ID NOs: 19-21.

The CDR sequences for 3G6, and 3G6 derived antibodies include CDRs of the heavy chain variable domain, SEQ ID NOs: 16-18, and CDRs of the light chain variable domain, SEQ ID NOs: 1-3, as defined by Kabat numbering. Under Chothia numbering, the CDR sequences of the heavy chain variable domain are set forth in SEQ ID NOs: 34-36, and CDRs of the light chain variable domain are set forth in SEQ ID NOs: 19-21.

The amino acid sequence for the heavy chain variable region of 1B2 antibody is set forth in SEQ ID NO: 38. The amino acid sequence for the light chain variable region of 1B2 antibody is set forth in SEQ ID NO: 37.

The amino acid sequence for the heavy chain variable region of 1C5 antibody is set forth in SEQ ID NO: 39. The amino acid sequence for the light chain variable region of 1C5 antibody is set forth in SEQ ID NO: 37.

The amino acid sequence for the heavy chain variable region of 1D6 antibody is set forth in SEQ ID NO: 40. The amino acid sequence for the light chain variable region of 1D6 antibody is set forth in SEQ ID NO: 37.

The amino acid sequence for the heavy chain variable region of 3E1 antibody is set forth in SEQ ID NO: 41. The amino acid sequence for the light chain variable region of 3E1 antibody is set forth in SEQ ID NO: 37.

The amino acid sequence for the heavy chain variable region of 3G6 antibody is set forth in SEQ ID NO: 42. The amino acid sequence for the light chain variable region of 3G6 antibody is set forth in SEQ ID NO: 37.

In some embodiments, the heavy chain variable region is at least 80%, 85%, 90%, 91%, 92%, 93%, 94%, 95%, 96%, 97%, 98%, or 99% identical to any one of SEQ ID NOs: 38-42. In some embodiments, the light chain variable region is at least 80%, 85%, 90%, 91%, 92%, 93%, 94%, 95%, 96%, 97%, 98%, or 99% identical to SEQ ID NO: 37. The heavy chain variable region sequence can be paired with the corresponding light chain variable region sequence, and together they bind to HER3.

Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects. In some embodiments, the variable regions are fully human, e.g., derived from human heavy chain immunoglobulin locus sequences (e.g., recombination of human IGHV, human IGHD, and human IGHJ genes), and/or human kappa chain immunoglobulin locus sequences (e.g., recombination of human IGKV and human IGKJ genes).

Furthermore, in some embodiments, the antibodies or antigen-binding fragments thereof described herein can also contain one, two, or three heavy chain variable region CDRs selected from the group of SEQ ID NOs: 4-6, SEQ ID NOs: 7-9, SEQ ID NOs: 10-12, SEQ ID NOs: 13-15, SEQ ID NOS: 16-18, SEQ ID NOs: 22-24, SEQ ID NOs: 25-27, SEQ ID NOs: 28-30, SEQ ID NOs: 31-33, SEQ ID NOs: 34-36; and/or one, two, or three light chain variable region CDRs selected from the group of SEQ ID NOs: 1-3 and SEQ ID NOs: 19-21.

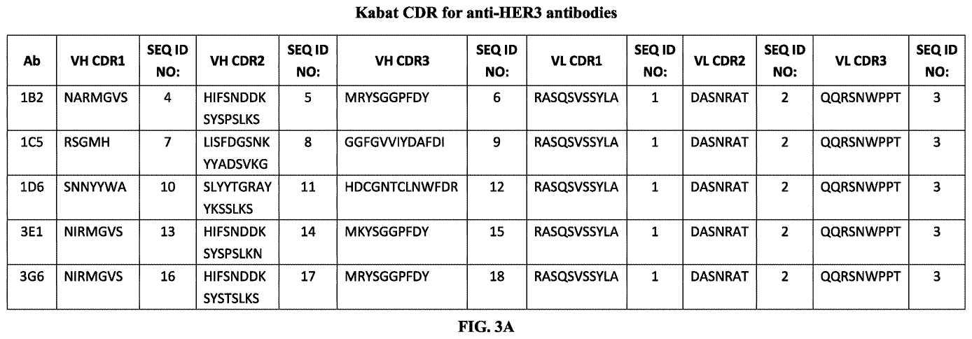

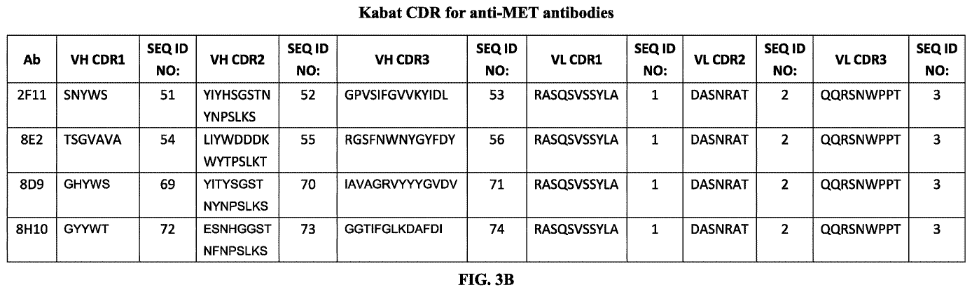

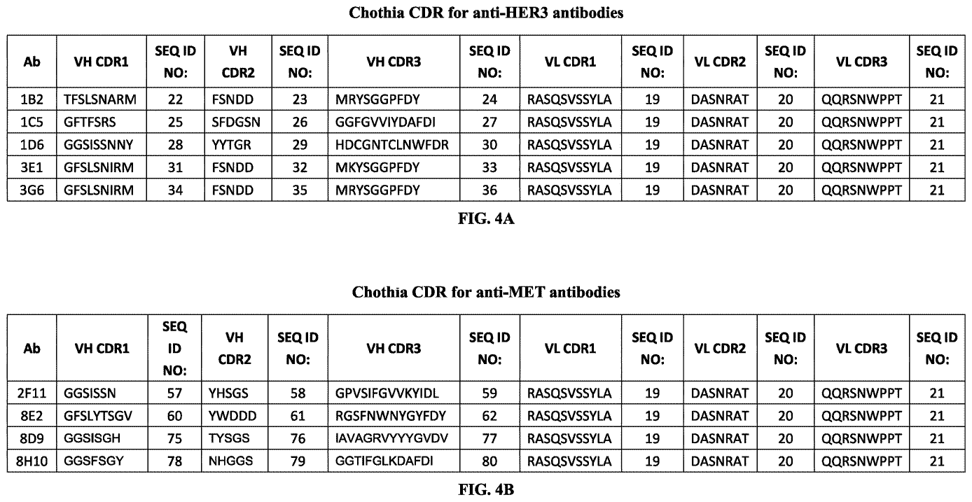

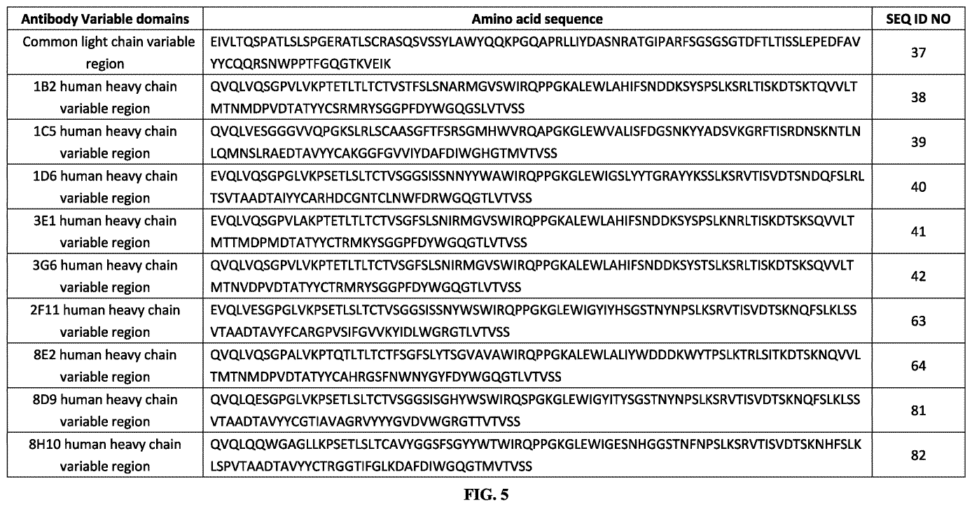

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibody can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 3A (Kabat CDR) and FIG. 4A (Chothia CDR).

In some embodiments, the antibody or an antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a light chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination of Kabat and Chothia numbering scheme.

The disclosure also provides antibodies or antigen-binding fragments thereof that bind to HER3. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 38, and the selected VL sequence is SEQ ID NO: 37. In some embodiments, the selected VH sequence is SEQ ID NO: 39 and the selected VL sequence is SEQ ID NO: 37. In some embodiments, the selected VH sequence is SEQ ID NO: 40, and the selected VL sequence is SEQ ID NO: 37. In some embodiments, the selected VH sequence is SEQ ID NO: 41, and the selected VL sequence is SEQ ID NO: 37. In some embodiments, the selected VH sequence is SEQ ID NO: 42, and the selected VL sequence is SEQ ID NO: 37.

The disclosure also provides antibodies or antigen-binding fragments thereof that can compete with the antibodies described herein. In some aspects, the antibodies or antigen-binding fragments can bind to the same epitope as the antibodies described herein.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 3A or FIG. 4A, or comprises sequences as shown in FIG. 5. When the polypeptides are paired with corresponding polypeptide (e.g., a corresponding heavy chain variable region or a corresponding light chain variable region), the paired polypeptides bind to HER3 (e.g., human HER3).

The anti-HER3 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to HER3 will retain an ability to bind to HER3. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

In some embodiments, the antibody or antigen-binding fragment thereof described herein recognizes an endogenous HER3 or a recombinant HER3. In some embodiments, the antibody or antigen-binding fragment thereof described herein recognizes human HER3.

In some embodiments, the half-life of the antibody or antigen-binding fragment thereof described herein in wild-type mice (e.g., C57BL/6 mice) is at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days. In some embodiments, the half-life of the antibody or antigen-binding fragment thereof described herein in HER3 gene humanized mice (e.g., hHER3 mice) is at least 1 day, at least 2 days, at least 3 days, at least 4 days, or at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days. In some embodiments, the max concentration (Cmax) of the antibody or antigen-binding fragment thereof described herein in HER3 gene humanized mice (e.g., hHER3 mice) is at least 50 μg/mL, at least 75 μg/mL, at least 100 μg/mL, at least 125 μg/mL, at least 150 μg/mL, at least 175 μg/mL, or at least 200 μg/mL. In some embodiments, the serum clearance rate (CL) of the antibody or antigen-binding fragment thereof described herein in HER3 gene humanized mice (e.g., hHER3 mice) is at least 7.5 mL/day/kg, at least 10 mL/day/kg, at least 12.5 mL/day/kg, at least 15 mL/day/kg, at least 17.5 mL/day/kg, at least 20 mL/day/kg, at least 25 mL/day/kg, at least 30 mL/day/kg, at least 35 mL/day/kg, or at least 40 mL/day/kg.

In some embodiments, the clearance rate (CL) of the antibody or antigen-binding fragment thereof described herein in wild-type mice (e.g., C57BL/6 mice) is less than 7 mL/day/kg, less than 6 mL/day/kg, less than 5 mL/day/kg, or less than 4 mL/day/kg. In some embodiments, the clearance rate (CL) of the antibody or antigen-binding fragment thereof described herein in HER3 gene humanized mice (e.g., hHER3 mice) is less than 15 mL/day/kg, less than 14 mL/day/kg, less than 13 mL/day/kg, or less than 12 mL/day/kg.

In some embodiments, the half-life of the antibody or antigen-binding fragment thereof described herein (e.g., in FcRn gene humanized mice) is at least 6 days, at least 7 days, at least 8 days, at least 9 days, at least 10 days, at least 11 days, at least 12 days, at least 13 days, or at least 14 days. In some embodiments, the clearance rate (CL) of the antibody or antigen-binding fragment thereof described herein (e.g., in FcRn gene humanized mice) is less than 16 mL/day/kg, less than 15 mL/day/kg, less than 14 mL/day/kg, less than 13 mL/day/kg, less than 12 mL/day/kg, less than 11 mL/day/kg, less than 10 mL/day/kg, less than 9 mL/day/kg, less than 8 mL/day/kg, or less than 7 mL/day/kg.

### Anti-MET Antibodies and Antigen-Binding Fragments

The disclosure also provides antibodies and antigen-binding fragments thereof that specifically bind to MET (e.g., human MET). The antibodies and antigen-binding fragments described herein are capable of binding to MET. These antibodies can be agonists or antagonists. In some embodiments, these antibodies can block MET activity.

The disclosure provides e.g., anti-MET antibodies 2F11 and 8E2, the chimeric antibodies thereof, and the human or humanized antibodies thereof.

The CDR sequences for 2F11, and 2F11 derived antibodies (e.g., human or humanized antibodies) include CDRs of the heavy chain variable domain, SEQ ID NOs: 51-53, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 57-59 and CDR sequences of the light chain variable domain are set forth in SEQ ID NOs: 19-21.

The CDR sequences for 8E2, and 8E2 derived antibodies include CDRs of the heavy chain variable domain, SEQ ID NOs: 54-56, and CDRs of the light chain variable domain, SEQ ID NOs: 1-3, as defined by Kabat numbering. Under Chothia numbering, the CDR sequences of the heavy chain variable domain are set forth in SEQ ID NOs: 60-62, and CDRs of the light chain variable domain are set forth in SEQ ID NOs: 19-21.

The CDR sequences for 8D9, and 8D9 derived antibodies include CDRs of the heavy chain variable domain, SEQ ID NOs: 69-71, and CDRs of the light chain variable domain, SEQ ID NOs: 1-3, as defined by Kabat numbering. Under Chothia numbering, the CDR sequences of the heavy chain variable domain are set forth in SEQ ID NOs: 75-77, and CDRs of the light chain variable domain are set forth in SEQ ID NOs: 19-21.

The CDR sequences for 8H10, and 8H10 derived antibodies include CDRs of the heavy chain variable domain, SEQ ID NOs: 72-74, and CDRs of the light chain variable domain, SEQ ID NOs: 1-3, as defined by Kabat numbering. Under Chothia numbering, the CDR sequences of the heavy chain variable domain are set forth in SEQ ID NOs: 78-80, and CDRs of the light chain variable domain are set forth in SEQ ID NOs: 19-21.

The amino acid sequence for the heavy chain variable region of 2F11 antibody is set forth in SEQ ID NO: 63. The amino acid sequence for the light chain variable region of 2F11 antibody is set forth in SEQ ID NO: 37.

The amino acid sequence for the heavy chain variable region of 8E2 antibody is set forth in SEQ ID NO: 64. The amino acid sequence for the light chain variable region of 8E2 antibody is set forth in SEQ ID NO: 37.

The amino acid sequence for the heavy chain variable region of 8D9 antibody is set forth in SEQ ID NO: 81. The amino acid sequence for the light chain variable region of 8D9 antibody is set forth in SEQ ID NO: 37.

The amino acid sequence for the heavy chain variable region of 8H10 antibody is set forth in SEQ ID NO: 82. The amino acid sequence for the light chain variable region of 8H10 antibody is set forth in SEQ ID NO: 37.

In some embodiments, the heavy chain variable region is at least 80%, 85%, 90%, 91%, 92%, 93%, 94%, 95%, 96%, 97%, 98%, or 99% identical to any one of SEQ ID NOs: 63, 64, 81 or 82. In some embodiments, the light chain variable region is at least 80%, 85%, 90%, 91%, 92%, 93%, 94%, 95%, 96%, 97%, 98%, or 99% identical to SEQ ID NO: 37. The heavy chain variable region sequence can be paired with the corresponding light chain variable region sequence, and together they bind to MET.

In some embodiments, the variable regions have a humanization percentage that is is greater than 80%, 81%, 82%, 83%, 84%, 85%, 86%, 87%, 88%, 89%, 90%, 91%, 92%, 93%, 94%, or 95%. In some embodiments, the variable regions are fully human, e.g., derived from human heavy chain immunoglobulin locus sequences (e.g., recombination of human IGHV, human IGHD, and human IGHJ genes), and/or human kappa chain immunoglobulin locus sequences (e.g., recombination of human IGKV and human IGKJ genes).

Furthermore, in some embodiments, the antibodies or antigen-binding fragments thereof described herein can also contain one, two, or three heavy chain variable region CDRs selected from the group of SEQ ID NOs: 51-53, SEQ ID NOs: 54-56, SEQ ID NOs: 57-59, SEQ ID NOs: 60-62, SEQ ID NOs: 69-71, SEQ ID NOs: 72-74, SEQ ID NOs: 75-77, SEQ ID NOs: 79-80; and/or one, two, or three light chain variable region CDRs selected from the group of SEQ ID NOs: 1-3 and SEQ ID NOs: 19-21.

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibody can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 3B (Kabat CDR) and FIG. 4B (Chothia CDR).

In some embodiments, the antibody or an antigen-binding fragment described herein can contain a heavy chain variable domain containing one, two, or three of the CDRs of SEQ ID NO: 51 with zero, one or two amino acid insertions, deletions, or substitutions; SEQ ID NO: 52 with zero, one or two amino acid insertions, deletions, or substitutions; SEQ ID NO: 53 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 54 with zero, one or two amino acid insertions, deletions, or substitutions; SEQ ID NO: 55 with zero, one or two amino acid insertions, deletions, or substitutions; SEQ ID NO: 56 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 57 with zero, one or two amino acid insertions, deletions, or substitutions; SEQ ID NO: 58 with zero, one or two amino acid insertions, deletions, or substitutions; SEQ ID NO: 59 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 60 with zero, one or two amino acid insertions, deletions, or substitutions; SEQ ID NO: 61 with zero, one or two amino acid insertions, deletions, or substitutions; SEQ ID NO: 62 with zero, one or two amino acid insertions, deletions, or substitutions.

In some embodiments, the antibody or an antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a light chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination of Kabat and Chothia numbering scheme.

The disclosure also provides antibodies or antigen-binding fragments thereof that bind to MET. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 63, and the selected VL sequence is SEQ ID NO: 37. In some embodiments, the selected VH sequence is SEQ ID NO: 64 and the selected VL sequence is SEQ ID NO: 37. In some embodiments, the selected VH sequence is SEQ ID NO: 81 and the selected VL sequence is SEQ ID NO: 37. In some embodiments, the selected VH sequence is SEQ ID NO: 82 and the selected VL sequence is SEQ ID NO: 37.

The disclosure also provides antibodies or antigen-binding fragments thereof that can compete with the antibodies described herein. In some aspects, the antibodies or antigen-binding fragments can bind to the same epitope as the antibodies described herein.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 3B or FIG. 4B, or comprises sequences as shown in FIG. 5. When the polypeptides are paired with corresponding polypeptide (e.g., a corresponding heavy chain variable region or a corresponding light chain variable region), the paired polypeptides bind to MET (e.g., human MET).

The anti-MET antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to MET will retain an ability to bind to MET. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

In some embodiments, the antibody or antigen-binding fragment thereof described herein recognizes an endogenous MET or a recombinant MET. In some embodiments, the antibody or antigen-binding fragment thereof described herein recognizes human MET.

### Antibodies and Antigen Binding Fragments

The present disclosure provides anti-HER3 antibodies and antigen-binding fragments thereof (including anti-HER3 multispecific antibodies and antigen-binding fragments thereof, e.g., anti-HER3/MET bispecific antibodies and antigen-binding fragments thereof). The present disclosure also provides anti-MET antibodies and antigen-binding fragments thereof (including anti-MET multispecific antibodies and antigen-binding fragments thereof, e.g., anti-HER3/MET bispecific antibodies and antigen-binding fragments thereof).

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68 (1-3): 9-16 (October 1997); Morea et al., J Mol Biol. 275 (2): 269-94 (January 1998); Chothia et al., Nature 342 (6252): 877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFV has one heavy chain variable domain, and one light chain variable domain. In some embodiments, the scFV has two heavy chain variable domains, and two light chain variable domains.

In some embodiments, sequences (e.g., CDRs or VH/VL sequences) of the antibody or antigen-binding fragment thereof described herein can be used to generate a bispecific antibody targeting HER3 and an addition antigen (e.g., EGFR).

Single-chain Fv (scFv) or antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

One-armed antibodies can have a heavy chain and a light chain, and a heavy chain fragment comprising CH2 and CH3 domains of IgG. In some embodiments, a one-armed antibody is an antibody that only has one of the two antigen binding arms in a typical antibody. In some embodiments, a one-armed antibody comprises an antigen binding arm (e.g., VH+CH1 and VL+CL), and a Fc.

Antibodies and antibody fragments of the present disclosure can be modified in the Fc region to provide desired effector functions or serum half-life. In some embodiments, the Fc region can be modified to silence or decrease complement-dependent cytotoxicity (CDC) or antibody-dependent cellular cytotoxicity (ADCC). In some embodiments, the Fc region can be modified to increase complement-dependent cytotoxicity (CDC) or antibody-dependent cellular cytotoxicity (ADCC).

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94:7509-7514, 1997). Antibody homodimers can be converted to Fab′2 homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

### Multispecific Antibodies and Antigen-Binding Fragments

The disclosure provides multispecific (e.g., bispecific) antibodies and antigen-binding fragments thereof that specifically bind to HER3 (e.g., human HER3). In one aspect, the disclosure provides an anti-HER3 multispecific (e.g., bispecific) antibody or antigen-binding fragment thereof, comprising: a first antigen-binding domain that specifically binds to HER3; and a second antigen-binding domain that specifically binds to a different antigen.

In one aspect, the disclosure provides an anti-HER3 multispecific (e.g., bispecific) antibody or antigen-binding fragment thereof, comprising: a first antigen-binding domain that specifically binds to a first epitope of HER3; and a second antigen-binding domain that specifically binds to a second epitope of HER3. In some embodiments, the first epitope is different from the second epitope.

In some embodiments, the first antigen-binding domain specifically binds to HER3; and the second antigen-binding domain specifically binds to MET.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively;
(7) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 19-21, respectively;
(8) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 19-21, respectively;
(9) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively; and
(10) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 4-6, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 10-12, respectively, and the selected VL2 CDRs 1, 2, 3 amino acid sequences are set forth in SEQ ID NOs: 1-3, respectively;
(4) the selected VH2 CDRs 1, 2, 3 amino acid sequences are set forth in SEQ ID NOs: 13-15, respectively, and the selected VL2 CDRs 1, 2, 3 amino acid sequences are set forth in SEQ ID NOs: 1-3, respectively;
(5) the selected VH2 CDRs 1, 2, 3 amino acid sequences are set forth in SEQ ID NOs: 16-18, respectively, and the selected VL2 CDRs 1, 2, 3 amino acid sequences are set forth in SEQ ID NOs: 1-3, respectively;
(6) the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 19-21, respectively;
(7) the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively;
(8) the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 19-21, respectively;
(9) the selected VH2 CDRs 1, 2, 3 amino acid sequences are set forth in SEQ ID NOs: 31-33, respectively, and the selected VL2 CDRs 1, 2, 3 amino acid sequences are set forth in SEQ ID NOs: 19-21, respectively; and
(10) the selected VH2 CDRs 1, 2, 3 amino acid sequences are set forth in SEQ ID NOs: 34-36, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively.

In some embodiments, the first heavy chain variable region comprises a sequence that is at least 80%, 85%, 90%, 95%, 99%, or 100% identical to SEQ ID NO: 41, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 39, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 42, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 39, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 38, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 39, and the second light chain variable region comprises a sequence that is at least 80%, 85%, 90%, 95%, 99%, or 100% identical to SEQ ID NO: 37.

In some embodiments, the VH1 comprises an amino acid sequence that is at least 80%, 85%, 90%, 95%, 99%, or 100% identical to a selected VH sequence, and the VL1 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In some embodiments, the VH2 comprises an amino acid sequence that is at least 80%, 85%, 90%, 95%, 99%, or 100% identical to a selected VH sequence, and the VL2 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In some embodiments, the first antigen-binding domain specifically binds to human, mouse, monkey, or dog HER3; and/or the second antigen-binding domain specifically binds to human, mouse, monkey, or dog HER3.

In some embodiments, the first antigen-binding domain is a human or humanized antigen-binding domain; and/or the second antigen-binding domain is a human or humanized antigen-binding domain.

In some embodiments, the first antigen-binding domain is a single-chain variable fragment (scFv); and/or the second antigen-binding domain is a scFv.

In some embodiments, the first light chain variable region and the second light chain variable region are identical.

In some embodiments, knobs-into-holes mutations were introduced in the Fc regions of the bispecific antibodies to reduce the chance of wrong pairing between the two heavy chains. Exemplary bispecific antibodies obtained include: 1C5-1B2, 1D6-1B2, 3E1-1B2, 3G6-1B2, 1D6-1C5, 3E1-1C5, 3G6-1C5, 3E1-1D6, 3G6-1D6, 3G6-3E1, 2F11-3E1, 2F11-3G6, 2F11-1B2, 2F11-1B2, 2F11-1C5 (FIG. 9). The sequence of the human IgG1 constant region with knob mutations is set forth in SEQ ID NO: 49, and the human IgG1 constant region with hole mutations is set forth in SEQ ID NO: 50.

In some embodiments, the anti-HER3 antibodies, bispecific antibodies, or antibody fragments thereof include the combinations of anti-HER3 antigen-binding domains shown in FIG. 9. In some embodiments, the first anti-HER3 antigen-binding domain comprises the CDRs of an anti-HER3 antibody as indicated in the first row of FIG. 9. In some embodiments, the second anti-HER3 antigen-binding domain comprises the CDRs of an anti-HER3 antibody as indicated in the first column of FIG. 9. In some embodiments, the first anti-HER3 antigen-binding domain comprises the VH and VL of an anti-HER3 antibody as indicated in the first row of FIG. 9. In some embodiments, the second anti-HER3 antigen-binding domain comprises the VH and VL of an anti-HER3 antibody as indicated in the first column of FIG. 9. For example, 3E1-1C5 refers to a bispecific anti-HER3 antibody that contains a first anti-HER3 antigen-binding domain that is derived from 3E1 and a second anti-HER3 antigen-binding domain that is derived from 1C5. In some embodiments, the first anti-HER3 antigen-binding domain comprises the CDRs of 3E1. In some embodiments, the second anti-HER3 antigen-binding domain comprises the CDRs of 1C5. In some embodiments, the first anti-HER3 antigen-binding domain comprises the VH and VL of 3E1. In some embodiments, the second anti-HER3 antigen-binding domain comprises the VH and VL of 1C5.

In some embodiments, in 3E1-1C5, the heavy chain constant region of 3E1 includes knob mutations, and the heavy chain constant region of 1C5 includes hole mutations. In some embodiments, in 3G6-1C5, the heavy chain constant region of 3G6 includes knob mutations, and the heavy chain constant region of 1C5 includes hole mutations. In some embodiments, in 1B2-1C5, the heavy chain constant region of 1B2 includes knob mutations, and the heavy chain constant region of 1C5 includes hole mutations.

### Anti-HER3/MET Bispecific Antibodies and Antigen-Binding Fragments

In some embodiments, as an example of the anti-HER3 multispecific antibodies and antigen-binding fragments, the present disclosure provides anti-HER3/MET bispecific antibodies and antigen-binding fragments.

In some embodiments, the anti-HER3/MET bispecific antibody or antigen-binding domain described herein has a common light chain. In some embodiments, the anti-HER3/MET bispecific antibody or antigen-binding domain has an anti-HER3 antigen-binding domain (e.g., 1B2, 1C5, 1D6, 3E1, or 3G6) and an anti-MET antigen-binding domain (e.g., 2F11, 8E2, 8D9 or 8H10). In some embodiments, the anti-HER3/MET bispecific antibody or antigen-binding domain has a heavy chain variable region targeting HER3 (e.g., any one of the VH targeting HER3 described herein), a heavy chain variable region targeting MET (e.g., any one of the VH targeting MET described herein), and two identical common light chain variable regions.

In some embodiments, the anti-HER3/MET bispecific antibodies or antigen-binding fragments thereof can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 3A-3B (Kabat CDR) and 4A-4B (Chothia CDR).

In some embodiments, the anti-HER3/MET bispecific antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence and the selected VL sequences are shown in FIG. 5.

In some embodiments, the anti-HER3/MET bispecific antibody or antigen-binding domain has any of the combinations of (1) heavy chain variable region targeting HER3, and (2) heavy chain variable region targeting MET as shown in FIG. 9. In some embodiments, the anti-HER3/MET bispecific antibody or antigen-binding domain has any of the below combination (1) heavy chain variable region targeting HER3, and (2) heavy chain variable region targeting MET: 2F11-3E1, 2F11-3G6, 2F11-1B2, 2F11-1C5, 8E2-1B2, 8E2-1C5, 8E2-3E1, 8E2-3G6, 3E1-8D9, 1B2-8D9, 1C5-8D9, 3E1-8H10, 1B2-8H10, or 1C5-8H10.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively;
(7) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 19-21, respectively;
(8) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 19-21, respectively;
(9) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively; and
(10) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 19-21, respectively;
(4) the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 19-21, respectively;
(5) the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(6) the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively;
(7) the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 19-21, respectively; and
(8) the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 19-21, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively.

In some embodiments, the first heavy chain variable region comprises a sequence that is at least 80%, 85%, 90%, 95%, 99%, or 100% identical to SEQ ID NO: 38, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 63, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 39, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 63, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 40, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 63, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 41, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 63, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 42, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 63, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 38, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 64, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 39, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 64, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 40, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 64, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 41, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 64, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 42, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 64, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 38, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 81, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 39, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 81, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 41, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 81, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 38, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 82, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 39, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 82, and the second light chain variable region comprises a sequence that is at least 80%, 85%, 90%, 95%, 99%, or 100% identical to SEQ ID NO: 37.

In some embodiments, the first heavy chain variable region comprises a sequence that is at least 80%, 85%, 90%, 95%, 99%, or 100% identical to SEQ ID NO: 41, the first light chain variable region comprises a sequence that is at least 80%, 85%, 90%, 95%, 99%, or 100% identical to SEQ ID NO: 37, the second heavy chain variable region comprises a sequence that is at least 80%, 85%, 90%, 95%, 99%, or 100% identical to SEQ ID NO: 82, and the second light chain variable region comprises a sequence that is at least 80%, 85%, 90%, 95%, 99%, or 100% identical to SEQ ID NO: 37.

In some embodiments, the VH1 comprises an amino acid sequence that is at least 80%, 85%, 90%, 95%, 99%, or 100% identical to a selected VH sequence, and the VL1 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

In some embodiments, the VH2 comprises an amino acid sequence that is at least 80%, 85%, 90%, 95%, 99%, or 100% identical to a selected VH sequence, and the VL2 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 63, and the selected VL sequence is SEQ ID NO: 37;
(2) the selected VH sequence is SEQ ID NO: 64, and the selected VL sequence is SEQ ID NO: 37;
(3) the selected VH sequence is SEQ ID NO: 81, and the selected VL sequence is SEQ ID NO: 37; and
(4) the selected VH sequence is SEQ ID NO: 82, and the selected VL sequence is SEQ ID NO: 37.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37. In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 63, and the selected VL sequence is SEQ ID NO: 37;
(2) the selected VH sequence is SEQ ID NO: 64, and the selected VL sequence is SEQ ID NO: 37;
(3) the selected VH sequence is SEQ ID NO: 81, and the selected VL sequence is SEQ ID NO: 37; and
(4) the selected VH sequence is SEQ ID NO: 82, and the selected VL sequence is SEQ ID NO: 37.

In some embodiments, the first antigen-binding domain specifically binds to human, mouse, monkey, or dog HER3; and/or the second antigen-binding domain specifically binds to human, mouse, monkey, or dog MET.

In some embodiments, the first antigen-binding domain is a human or humanized antigen-binding domain; and/or the second antigen-binding domain is a human or humanized antigen-binding domain.

In some embodiments, the first antigen-binding domain is a single-chain variable fragment (scFv); and/or the second antigen-binding domain is a scFv.

In some embodiments, the first light chain variable region and the second light chain variable region are identical.

### Antibody Drug Conjugates (ADC)

In some embodiments, the antibodies, the antigen-binding fragments thereof, or the bispecific antibodies described herein can be conjugated to a therapeutic agent, optionally with a linker, to form an antibody-drug conjugate. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., monomethyl auristatin E, monomethyl auristatin F, camptothecin, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). In some embodiments, the therapeutic agent is MMAE or MMAF.

Definitions of specific functional groups and chemical terms are described in more detail below. For purpose of this invention, the chemical elements are identified in accordance with the Periodic Table of the Elements, CAS version, Handbook of Chemistry and Physics, 75th Edition, inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modem Methods of Organic Synthesis, 3rd Edition, Cambridge University Press, Cambridge, 1987.

All ranges cited herein are inclusive, unless expressly stated to the contrary. When a range of values is listed, it is intended to encompass each value and sub-range within the range. For example, “C1-6” is intended to encompass, C1, C2, C3, C4, C5, C6, C1-6, C1-5, C1-4, C1-3, C1-2, C2-6, C2-5, C2-4, C2-3, C3-6, C3-5, C3-4, C4-6, C4-5, and C5-6.

The compounds or any formula depicting and describing the compounds of the present disclosure may have one or more chiral (asymmetric) centers. The present invention encompasses all stereoisomeric forms of the compounds or any formula depicting and describing the compounds of the present invention. Centers of asymmetry that are present in the compounds or any formula depicting and describing the compounds of the present invention can all independently of one another have (R) or(S) configuration. When bonds to a chiral carbon are depicted as straight lines in the structural formulas, or when a compound name is recited without an (R) or(S) chiral designation for a chiral carbon, it is understood that both the (R) and(S) configurations of each such chiral carbon, and hence each enantiomer or diastereomer and mixtures thereof, are embraced within the formula or by the name.

The disclosure includes all possible enantiomers and diastereomers and mixtures of two or more stereoisomers, for example mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism the disclosure includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis.

Unless otherwise stated, the structures depicted herein are also meant to include the compounds that differ only in the presence of one or more isotopically enriched atoms, in other words, the compounds wherein one or more atoms are replaced by atoms having the same atomic number, but an atomic mass or mass number different from the atomic mass or mass number which predominates in nature. Such compounds are referred to as a “isotopic variant”. The present disclosure is intended to include all pharmaceutically acceptable isotopic variants of the compounds or any formula depicting and describing the compounds of the present invention. Examples of isotopes suitable for inclusion in the compounds of the present invention include, but not limited to, isotopes of hydrogen, such as 2H (i.e., D) and 3H; carbon, such as 11C, 13C, and 14C; chlorine, such as 36Cl; fluorine, such as 18F; iodine, such as 123I and 125I; nitrogen, such as 13N and 15N; oxygen, such as 150, 170, and 180; phosphorus, such as 32P; and sulfur, such as 35S. Certain isotopic variants of the compounds or any formula depicting and describing the compounds of the present disclosure, for example those incorporating a radioactive isotope, may be useful in drug and/or substrate tissue distribution studies. Particularly, compounds having the depicted structures that differ only in the replacement with heavier isotopes, such as the replacement of hydrogen by deuterium (2H, or D), can afford certain therapeutic advantages, for example, resulting from greater metabolic stability, increased in vivo half-life, or reduced dosage requirements and, hence, may be utilized in some particular circumstances. Isotopic variants of compounds or any formula depicting and describing the compounds of the present disclosure can generally be prepared by techniques known to those skilled in the art or by processes analogous to those described in the accompanying examples and synthesis using an appropriate isotopically-labeled reagent in place of the non-labeled reagent previously employed.

The compounds as provided herein are described with reference to both generic formulas and specific compounds. In addition, the compounds of the present disclosure may exist in a number of different forms or derivatives, all within the scope of the disclosure. These include, for example, pharmaceutically acceptable salts, tautomers, stereoisomers, racemic mixtures, regioisomers, prodrugs, solvated forms, different crystal forms or polymorphs, and active metabolites, etc.

As used herein, the term “pharmaceutically acceptable salt”, unless otherwise stated, includes salts that retain the biological effectiveness of the free acid/base form of the specified compound and that are not biologically or otherwise undesirable. Pharmaceutically acceptable salts may include salts formed with inorganic bases or acids and organic bases or acids. In cases where the compounds of the present disclosure contain one or more acidic or basic groups, the disclosure also comprises their corresponding pharmaceutically acceptable salts. Thus, the compounds of the present invention which contain acidic groups, such as carboxyl groups, can be present in salt form, and can be used according to the invention, for example, as alkali metal salts, alkaline earth metal salts, aluminum salts or as ammonium salts. More non-limiting examples of such salts include lithium salts, sodium salts, potassium salts, calcium salts, magnesium salts, barium salts, or salts with ammonia or organic amines such as ethylamine, ethanolamine, diethanolamine, triethanolamine, piperidine, N-methylglutamine, or amino acids. These salts are readily available, for instance, by reacting the compound having an acidic group with a suitable base, e.g., lithium hydroxide, sodium hydroxide, sodium propoxide, potassium hydroxide, potassium ethoxide, magnesium hydroxide, calcium hydroxide, or barium hydroxide. Other base salts of compounds of the present disclosure include but are not limited to copper (I), copper (II), iron (II), iron (III), manganese (II), and zinc salts. Compounds of the present disclosure which contain one or more basic groups, e.g., groups which can be protonated, can be present in salt form, and can be used according to the disclosure in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid, or aspartic acid, and other acids known to those skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates, and glutamates. The stoichiometry of the salts formed from the compounds of the disclosure may moreover be an integral or non-integral multiple of one.

Compounds of the present disclosure which contain basic nitrogen-containing groups can be quaternized using agents such as C1-4alkyl halides, for example, methyl, ethyl, isopropyl, and tert-butyl chloride, bromide, and iodide; diC1-4alkyl sulfates, for example, dimethyl, diethyl, and diamyl sulfate; C10-18alkyl halides, for example, decyl, dodecyl, lauryl, myristyl, and stearyl chloride, bromide, and iodide; and arylC1-4alkyl halides, for example, benzyl chloride and phenethyl bromide.

If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to those skilled in the art, for example by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts. For a review on more suitable salts, see Stahl and Wermuth, Handbook of Pharmaceutical Salts: Properties, Selection, and Use (Wiley-VCH, 2002).

The compound or any formula depicting and describing the compounds of the present disclosure and pharmaceutically acceptable salts thereof may exist in unsolvated and solvated forms. As used herein, the term “solvate” refers to a molecular complex comprising the compound of Formula (I), or a pharmaceutically acceptable salt thereof, and one or more pharmaceutically acceptable solvent molecules. For example, the term “hydrate” is employed when the solvent is water.

Pharmaceutically acceptable solvates in accordance with the present disclosure may include those wherein the solvent of crystallization may be isotopically substituted, e.g., D20, d6-acetone, d6-DMSO.

### Linker (Linking Agent Compound)

In some embodiments, the therapeutic agent is conjugated via a linker (or a linking agent compound). As used herein, the term “linker” or “linking agent compound” refers to a compound that can connect a ligand (e.g., the antibodies, the antigen-binding fragments thereof, or the antigen-binding protein constructs (e.g., bispecific antibodies) described herein) and a therapeutic agent (e.g., any of the therapeutic agents described herein) together to form a ligand-drug conjugate by reacting with a group of the ligand compound and the therapeutic agent compound respectively by, for example, a coupling reaction.

In some embodiments, the linker described herein is a compound having the following formula:

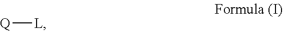

or a pharmaceutically acceptable salt, solvate, stereoisomer, or isotopic variant thereof, wherein Q denotes to a junction moiety capable of being coupled to a ligand via a bond selected from the group consisting of carbonyl, thioether, amide, disulfide and hydrazone bond; L denotes to a linker moiety capable of connecting Q to a therapeutic agent.

In some embodiments, the junction moiety (Q in Formula (I)) has the following structure:

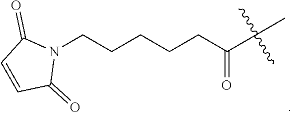 [SMILES: *C(=O)CCCCCN1C(=O)C=CC1=O]

In some embodiments, the linker moiety (L in Formula (I)) has the following formula:

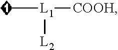

where L1 is a polypeptide residue consisting of three to eight amino acid residues which comprises at least one amino acid residue with a side chain carboxyl group, for example, glutamic acid residue or aspartic acid residue, where “—COOH” denotes carboxyl group of an amino acid residue at C-terminal of the polypeptide residue;
L2 is absent or a monodentate, bidentate or tridentate hydrophilic group attached to the side chain carboxyl group on the amino acid residue of the polypeptide residue L1, and L2 has a structure of —NHC(RL2a) (RL2b) (RL2c), where RL2a, RL2b, and RL2c are each independently selected from the group consisting of H, —(CH₂O) (CH₂CH₂O)m(CH₂)pC(O)OH, and —(CH₂O) (CH₂CH₂O)m(CH₂)pC(O)NHRL2d, RL2d is H or C1-6 alkyl optionally substituted with 1 to 6 hydroxy groups, each m is independently an integer from 0 to 10, preferably 0 to 4, for example 0, 1, 2, 3, or 4, especially preferably m is 0, and each p is independent an integer from 1 to 4, for example, 1, 2, 3, or 4; and
[symbol] denotes to the N-terminal side of the polypeptide residue covalently attached to the junction moiety Q.

In some embodiments, the polypeptide residue L1 is NH-Glu-Val-Ala-COOH. In some embodiments, the hydrophilic group L2 has the following structure:

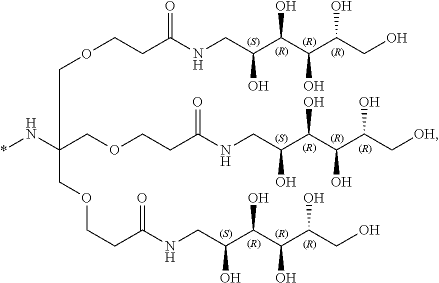

wherein “*” denotes the site covalently attached to polypeptide residue L1, e.g., side chain of the Glu residue in NH-Glu-Val-Ala-COOH.

In some embodiments, the linker described herein is a compound having the following structure:

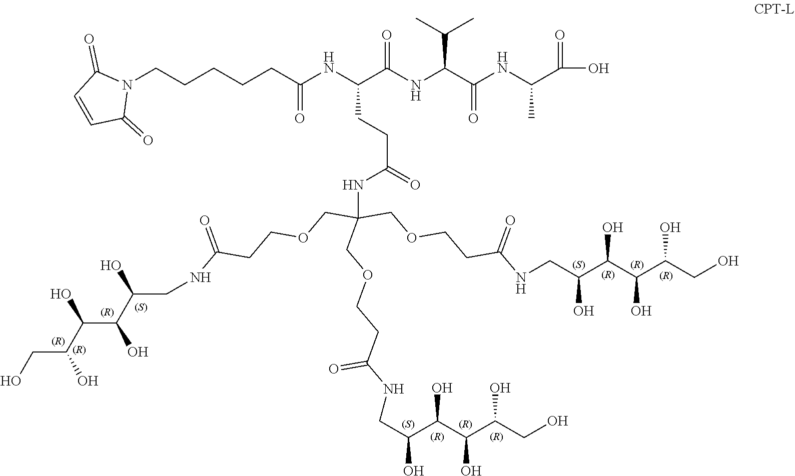

In some embodiments, the linker is a VC linker. Details of the linkers used for ADCs can be found, e.g., in Su, Z. et al. “Antibody-drug conjugates: Recent advances in linker chemistry.” Acta Pharmaceutica Sinica B (2021), which is incorporated herein by reference in its entirety.

### Therapeutic Agent

In some embodiments, the therapeutic agent that is conjugated to the antibodies, the antigen-binding fragments thereof, or the antigen-binding protein constructs (e.g., bispecific antibodies) described herein is discussed as follows.

In some embodiments, the therapeutic agent described herein is a cytotoxic agent. In some embodiments, the cytotoxic agent is a camptothecin compound, an analogue or a derivative thereof. In some preferred embodiments, the camptothecin compound is a compound having the following structure:

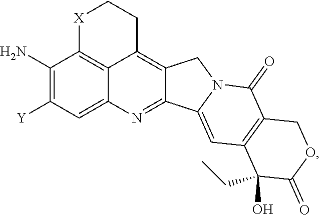

wherein X is selected from the group consisting of —CH2-, O and S; Y is selected from the group consisting of H, D, and F.

In some embodiments, the therapeutic agent is(S)-4-amino-9-ethyl-9-hydroxy-1,9,12,15-tetrahydro-13H-pyrano[3′,4′: 6,7]indolizino[1,2-b]thiopyrano[4,3,2-de]quinoline-10,13 (2H)-dione) (CPT-1). The structure of CPT-1 is shown below:

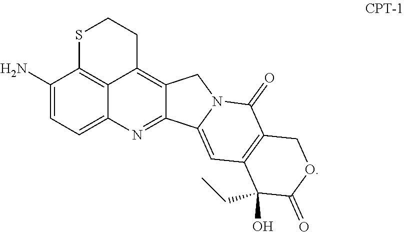 [SMILES: CC[C@@]1(O)C(=O)OCc2c1cc1n(c2=O)Cc2c-1nc1ccc(N)c3c1c2CCS3]

In some embodiments, the therapeutic agent is(S)-4-amino-9-ethyl-9-hydroxy-1,9,12,15-tetrahydro-13H-pyrano[4,3,2-de]pyrano[3′,4′: 6,7]indolizino[1,2-b]quinoline-10,13 (2H)-dione (CPT-2). The structure of CPT-2 is shown below:

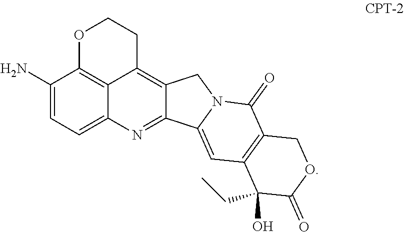 [SMILES: CC[C@@]1(O)C(=O)OCc2c1cc1n(c2=O)Cc2c-1nc1ccc(N)c3c1c2CCO3]

In some embodiments, the therapeutic agent is CPT3. The structure of CPT-3 is shown below:

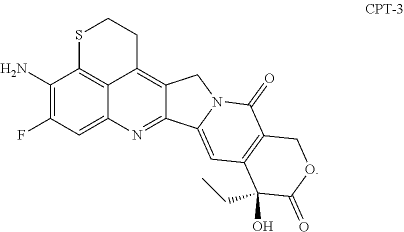 [SMILES: CC[C@@]1(O)C(=O)OCc2c1cc1n(c2=O)Cc2c-1nc1cc(F)c(N)c3c1c2CCS3]

In some embodiments, the therapeutic agent is(S)-4-amino-9-ethyl-5-fluoro-9-hydroxy-1,9,12,15-tetrahydro-13H-pyrano[4,3,2-de]pyrano[3′,4′: 6,7]indolizino[1,2-b]quinoline-10,13 (2H)-dione (CPT-4). The structure of CPT-4 is shown below:

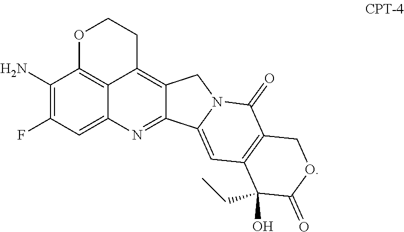 [SMILES: CC[C@@]1(O)C(=O)OCc2c1cc1n(c2=O)Cc2c-1nc1cc(F)c(N)c3c1c2CCO3]

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

### Linker-Therapeutic Agent Compound

In some embodiments, a linker (e.g., any of the linkers described herein) and a therapeutic agent (e.g., any of the therapeutic agents described herein) can be linked to form a “linker-therapeutic agent” compound.

In some embodiments, the linker-therapeutic agent compound has the following structure:

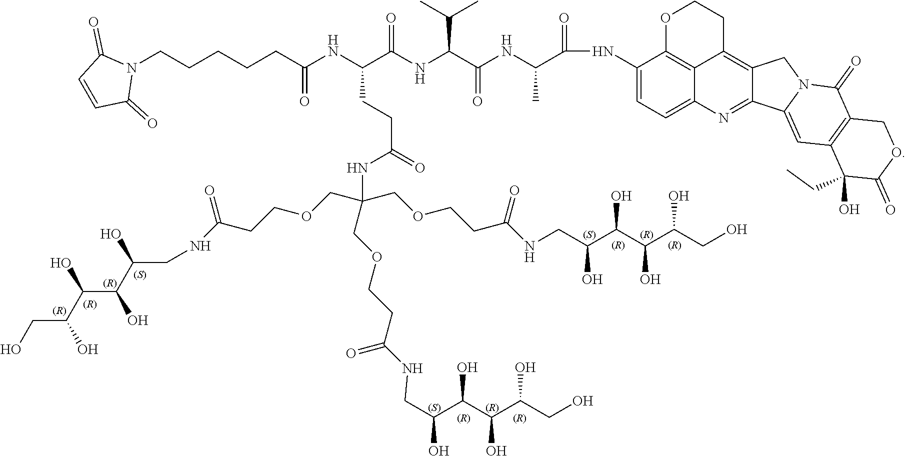

In some embodiments, the linker-therapeutic agent compound has the following structure:

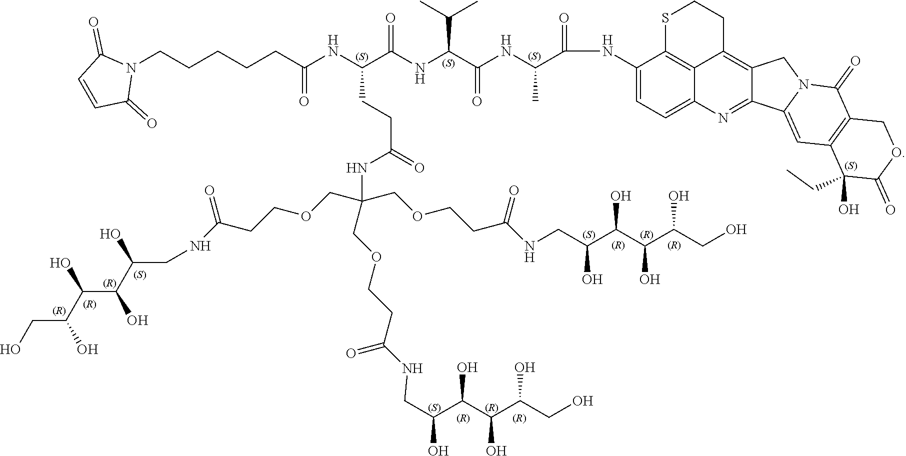

In some embodiments, an antibody (“Ab”), e.g., any of the antibodies, the antigen-binding fragments thereof, or the antigen-binding protein constructs (e.g., bispecific antibodies) described herein, can be linked to a linker-therapeutic agent compound (e.g., any of the linker-therapeutic agent compounds described herein) to generate an antibody-drug conjugate. In some embodiments, the antibody-drug conjugate has the following structure:

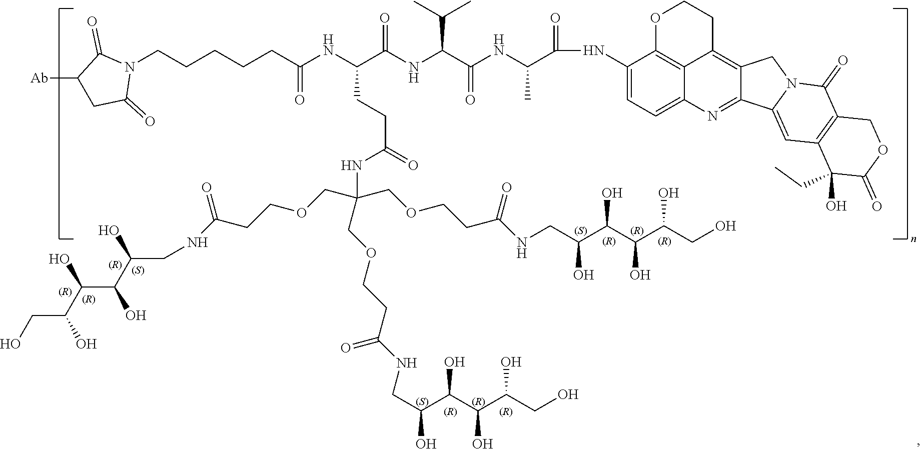

wherein n=1, 2, 3, 4, 5, 6, 7, or 8.

### Antibody and ADC Characteristics

In some embodiments, the antibodies or antigen-binding fragments thereof described herein or ADC derived therefrom can block the binding between HER3 and HER3 ligands (e.g., Heregulin).

The antibodies or antigen-binding fragments thereof as described herein or ADC derived therefrom can be an agonist or antagonist. In some embodiments, by binding to HER3, the antibody or antigen-binding fragment thereof can inhibit HER3 signaling pathway. In some embodiments, the antibody or antigen-binding fragment thereof can upregulate immune response or downregulate immune response.

In some embodiments, the antibody (or antigen-binding fragments thereof) or ADC derived therefrom specifically binds to HER3 (e.g., human HER3, monkey HER3 (e.g., rhesus macaques, Macaca fascicularis), dog HER3, mouse HER3) with a dissociation rate (koff) of less than 0.1 s⁻¹, less than 0.01 s⁻¹, less than 0.001 s⁻¹, less than 0.0001 s⁻¹, less than 0.00001 s⁻¹, less than 0.000001 s⁻¹ or less than 0.0000001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, greater than 0.000001 s⁻¹, greater than 0.0000001 s⁻¹ or greater than 0.00000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD-koff/kon). In some embodiments, KD is less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M or less than 1×10⁻¹⁴ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, greater than 1×10⁻¹² M, greater than 1×10⁻¹³ M, greater than 1×10⁻¹⁴ M.

General techniques for measuring the affinity of an antibody for an antigen include, e.g., ELISA, RIA, and surface plasmon resonance (SPR). In some embodiments, the antibody binds to human HER3 (SEQ ID NO: 43), monkey HER3 (SEQ ID NO: 44), dog HER3 (SEQ ID NO: 45), and/or mouse HER3 (SEQ ID NO: 46). In some embodiments, the antibody does not bind to human HER3, monkey HER3, dog HER3, and/or mouse HER3. In some embodiments, the cross-species binding of the antibody (or antigen-binding fragments thereof) or ADC derived therefrom can be measured by a cell binding assay. In some embodiments, the antibody or antigen-binding fragment thereof as described herein or ADC derived therefrom has a percentage of positive cells that is above 5%, above 10%, above 15%, above 20%, above 25%, above 30%, above 35%, above 40%, above 45%, above 50%, above 55%, above 60%, above 65%, above 70%, above 75%, above 80%, above 85%, above 90%, above 91%, above 92%, above 93%, above 94%, above 95%, above 96%, above 97%, or above 98%.

In some embodiments, the antibody (or antigen-binding fragments thereof) or ADC derived therefrom specifically binds to MET (e.g., human MET, monkey MET (e.g., rhesus macaques, Macaca fascicularis), dog MET, mouse MET) with a dissociation rate (koff) of less than 0.1 s⁻¹, less than 0.01 s⁻¹, less than 0.001 s⁻¹, less than 0.0001 s⁻¹, less than 0.00001 s⁻¹, less than 0.000001 s⁻¹ or less than 0.0000001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, greater than 0.000001 s⁻¹, greater than 0.0000001 s⁻¹ or greater than 0.00000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷M, less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M or less than 1×10⁻¹⁴ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, greater than 1×10⁻¹² M, greater than 1×10⁻¹³ M, greater than 1×10⁻¹⁴ M.

General techniques for measuring the affinity of an antibody for an antigen include, e.g., ELISA, RIA, and surface plasmon resonance (SPR). In some embodiments, the antibody binds to human MET (SEQ ID NO: 65), monkey MET (SEQ ID NO: 66), dog MET, and/or mouse MET. In some embodiments, the antibody does not bind to human MET, monkey MET, dog MET, and/or mouse MET. In some embodiments, the cross-species binding of the antibody (or antigen-binding fragments thereof) or ADC derived therefrom can be measured by a cell binding assay. In some embodiments, the antibody or antigen-binding fragment thereof as described herein or ADC derived therefrom has a percentage of positive cells that is above 5%, above 10%, above 15%, above 20%, above 25%, above 30%, above 35%, above 40%, above 45%, above 50%, above 55%, above 60%, above 65%, above 70%, above 75%, above 80%, above 85%, above 90%, above 91%, above 92%, above 93%, above 94%, above 95%, above 96%, above 97%, or above 98%.

In some embodiments, the antibody or antigen-binding fragment thereof as described herein or ADC derived therefrom is added to various cells, such as NUGC-4 cells (e.g., Cobioer, Cat #: CBP60493), LN229 cells (e.g., ATCC, Cat #: CRL-2611), MCF-7 cells, SNU-5 cells (e.g., ATCC, Cat #: CRL-5973), Hep3B cells (e.g., ATCC, Cat #: HB-8064), HepG2 cells (e.g., ATCC, Cat #: HB-8065), or PLC-PRF-5 cells (e.g., ATCC, Cat #: CRL-8024) to test the endocytosis rate. In some embodiments, after incubating for a period of time (e.g., 6 hours), the cells were centrifuged and washed in FACS buffer. MFI was detected on a flow cytometer, and the endocytosis rates of the antibodies were calculated. In some embodiments, the antibody or antigen-binding fragment thereof as described herein or ADC derived therefrom has an endocytosis rate of above 5%, above 10%, above 15%, above 20%, above 25%, above 30%, above 35%, above 40%, above 45%, above 50%, above 55%, above 60%, above 65%, above 70%, above 75%, above 80%, above 85%, above 90%, above 91%, above 92%, above 93%, above 94%, above 95%, above 96%, above 97%, or above 98%.

In some embodiments, thermal stabilities are determined. The antibodies or antigen binding fragments as described herein or ADC derived therefrom can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies or antigen-binding fragments thereof as described herein or ADC derived therefrom can bind to the same epitope of HER3. In some embodiments, the antibodies or antigen-binding fragments thereof as described herein or ADC derived therefrom can bind to different epitopes of HER3.

In some embodiments, the antibodies or antigen-binding fragments thereof as described herein have a purity of above 90%, above 91%, above 92%, above 93%, above 94%, above 95%, above 96%, above 97%, or above 98%, as determined by size exclusion chromatography (SEC). In some embodiments, the antibodies or antigen-binding fragments thereof as described herein have a hydrophobic interaction chromatography (HIC) retention time that is higher than 2 minutes, higher than 3 minutes, higher than 4 minutes, higher than 5 minutes, higher than 6 minutes, higher than 7 minutes, higher than 8 minutes, higher than 9 minutes, higher than 10 minutes, higher than 11 minutes, higher than 12 minutes, higher than 13 minutes, higher than 14 minutes, higher than 15 minutes, higher than 16 minutes, higher than 17 minutes, higher than 18 minutes, higher than 19 minutes, higher than 20 minutes, higher than 21 minutes or higher than 22 minutes.

In some embodiments, the antibodies or antigen-binding fragments thereof as described herein have a purity of purity of above 90%, above 91%, above 92%, above 93%, above 94%, above 95%, above 96%, above 97%, above 98%, or above 99%, as determined by capillary electrophoresis-sodium dodecyl sulphate (CE-SDS).

In some embodiments, the antibodies or antigen-binding fragments thereof as described herein have a main peak that constitutes more than 40%, more than 50%, more than 55%, more than 60%, more than 65%, more than 70%, more than 75%, more than 80%, more than 85%, or more than 90%, as determined by capillary isoelectric focusing (cIEF). In some embodiments, the antibodies or antigen-binding fragments thereof as described herein have an acidic peak that constitutes more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, or more than 90%, as determined by capillary isoelectric focusing (cIEF).

In some embodiments, the ADC described herein has an average drug-to-antibody ratio (DAR) of higher than 3, higher than 3.2, higher than 3.4, higher than 3.6, higher than 3.8, higher than 4, higher than 4.2, higher than 4.4, or higher than 4.6, as determined by HPLC. In some embodiments, the ADC described herein has an average DAR of lower than 3, lower than 3.2, lower than 3.4, lower than 3.6, lower than 3.8, lower than 4, lower than 4.2, lower than 4.4 or lower than 4.6, as determined by HPLC.

In some embodiments, the antibody or antigen-binding fragment thereof as described herein or ADC derived therefrom has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody or antigen-binding fragment thereof as described herein or ADC derived therefrom has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( Ti - T 0 ) / ( Vi - V 0 ) ] \times 100 \%$$

Ti is the average tumor volume in the treatment group on day i. TO is the average tumor volume in the treatment group on day zero. Vi is the average tumor volume in the control group on day i. V0 is the average tumor volume in the control group on day zero.

In some embodiments, the antibodies or antigen-binding fragments thereof as described herein or ADC derived therefrom are HER3 antagonist. In some embodiments, the antibodies or antigen binding fragments as described herein or ADC derived therefrom decrease HER3 signal transduction in a target cell that expresses HER3.

In some embodiments, the antibodies or antigen binding fragments as described herein or ADC derived therefrom can enhance APC (e.g., DC cell) function, for example, inducing surface expression of costimulatory and MHC molecules, inducing production of proinflammatory cytokines, and/or enhancing T cell triggering function.

In some embodiments, the antibodies or antigen binding fragments as described herein or ADC derived therefrom can bind to tumor cells that express HER3. In some embodiments, the antibodies or antigen binding fragments as described herein or ADC derived therefrom can induce complement-dependent cytotoxicity (CDC) and/or antibody dependent cellular cytoxicity (ADCC), and kill the tumor cell.

In some embodiments, the antibodies or antigen binding fragments as described herein or ADC derived therefrom have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis.

In some embodiments, the antibodies or antigen binding fragments as described herein or ADC derived therefrom can induce complement complement-dependent cytotoxicity (CDC).

In some embodiments, the Fc region is human IgG1, human IgG2, human IgG3, or human IgG4. In some embodiments, the antibody is a human IgG1 antibody, optionally with SI mutations, LALA mutations, N297A mutation, YTE mutations, and/or FLAA mutations. In some embodiments, the antibody is a human IgG4 antibody, optionally with SI mutations, LALA mutations, N297A mutation, YTE mutations, and/or FLAA mutations.

In some embodiments, the antibodies or antigen binding fragments as described herein or ADC derived therefrom do not have a functional Fc region. For example, the antibodies or antigen binding fragments are Fab, Fab′, F(ab′)₂, and Fv fragments. In some embodiments, the Fc region has LALA mutations (L234A and L235A mutations according to EU numbering), or LALA-PG mutations (L234A, L235A, P329G mutations according to EU numbering). In some embodiments, the Fc region has FLAA mutations (F234A and L235A according to EU numbering). In some embodiments, the Fc has SI mutations (S239D and I332E mutations according to EU numbering). In some embodiments, the Fc has N297A mutation according to EU numbering. In some embodiments, the Fc has YTE mutations (M252Y, S254T and T256E according to EU numbering).

### Methods of Making Antibodies

An isolated fragment of human protein (e.g., HER3 or MET3) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of HER3 or MET and encompasses an epitope of the protein such that an antibody raised against the peptide forms a specific immune complex with the protein. As described above, the full length sequence of human HER3 (SEQ ID NO: 43) is known in the art. In some embodiments, an Fc-tagged or His-tagged human HER3 protein is used as the immunogen. Similarly, the full length sequence of human MET is known in the art. In some embodiments, an Fc-tagged or His-tagged human MET protein is used as the immunogen.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human HER3 or MET). The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of HER3 or MET) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A or protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., HER3 or MET. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al., J. Immunol., 151:2296 (1993); Chothia et al., J. Mol. Biol., 196:901 (1987)).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric antibody will contain an amino acid sequence having at least 75%, 80%, 85%, 90%, 95%, 96%, 97%, 98%, or 99% percent identity with a sequence present in the light or heavy chain of the original antibody.

In some embodiments, a mouse (e.g., RenMab™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab™ mice can be found in PCT/CN2020/075698 or US20200390073A1, which is incorporated herein by reference in its entirety.

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chains. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

The antibodies generated by the mice have a full human VH, a full human VL, and mouse constant regions. In some embodiments, the human VH and human VL is linked to a human IgG constant regions (e.g., IgG1, IgG2, IgG3, and IgG4).

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

Additional modifications to the antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells. For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antigen-binding fragments thereof of the present disclosure (e.g., anti-HER3 antibody, anti-MET antibody, or anti-HER3/MET antibody) can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, esophageal cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the cancer is NSCLC, ovarian cancer, melanoma, colorectal cancer, breast cancer, a hematological malignancy, head and neck cancer, gastrointestinal cancer, bladder cancer, or bone cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors. In some embodiments, the cancer is colorectal cancer, gastric cancer, breast cancer, lung cancer, melanoma, ovarian cancer, head and neck cancer, pancreatic cancer, liver carcinoma or cervical cancer.

In some embodiments, the compositions and methods disclosed herein can be used for treatment of patients at risk for a cancer. Patients with cancer can be identified with various methods known in the art.

In one aspect, the disclosure provides methods for treating, preventing, or reducing the risk of developing disorders associated with an abnormal or unwanted immune response, e.g., an autoimmune disorder. These autoimmune disorders include, but are not limited to, Alopecia areata, lupus, ankylosing spondylitis, Meniere's disease, antiphospholipid syndrome, mixed connective tissue disease, autoimmune Addison's disease, multiple sclerosis, autoimmune hemolytic anemia, myasthenia gravis, autoimmune hepatitis, pemphigus vulgaris, Behcet's disease, pernicious anemia, bullous pemphigoid, polyarthritis nodosa, cardiomyopathy, polychondritis, celiac sprue-dermatitis, polyglandular syndromes, chronic fatigue syndrome (CFIDS), polymyalgia rheumatica, chronic inflammatory demyelinating, polymyositis and dermatomyositis, chronic inflammatory polyneuropathy, primary agammaglobulinemia, Churg-Strauss syndrome, primary biliary cirrhosis, cicatricial pemphigoid, psoriasis, CREST syndrome, Raynaud's phenomenon, cold agglutinin disease, Reiter's syndrome, Crohn's disease, Rheumatic fever, discoid lupus, rheumatoid arthritis, Cryoglobulinemia sarcoidosis, fibromyalgia, scleroderma, Grave's disease, Sjögren's syndrome, Guillain-Barre, stiff-man syndrome, Hashimoto's thyroiditis, Takayasu arteritis, idiopathic pulmonary fibrosis, temporal arteritis/giant cell arteritis, idiopathic thrombocytopenia purpura (ITP), ulcerative colitis, IgA nephropathy, uveitis, diabetes (e.g., Type I), vasculitis, lichen planus, and vitiligo. The antibodies or antigen-binding fragments thereof can also be administered to a subject to treat, prevent, or reduce the risk of developing disorders associated with an abnormal or unwanted immune response associated with cell, tissue or organ transplantation, e.g., renal, hepatic, and cardiac transplantation, e.g., graft versus host disease (GVHD), or to prevent allograft rejection. In some embodiments, the subject has dermatological disorders, liver disease (e.g., cirrhosis), Hidradenitis, experimental autoimmune encephalomyelitis. In some embodiments, the subject has renal disease, lupus, Sjogren's syndrome, ulcerative colitics, psoriasis, Hidradenitis suppurativa, Immune Thrombocytopenia (ITP), or other inflammatory arthritis. In some embodiments, the subject has multiple sclerosis or myasthenia gravis. In some embodiments, the subject has Crohn's disease, ulcerative colitis or type 1 diabetes. In some embodiments, the subject has autoimmune thyroid disease, Grave's disease, multiple sclerosis, psoriasis, inflammatory bowel disease (e.g., Crohn's Disease (CD) and ulcerative colitis), rheumatoid arthritis, Sjögren's syndrome, autoimmune nephritis, or systemic lupus erythematosus. In some embodiments, the methods involve administering to the subject an effective amount of a composition comprising the antibody or antigen-binding fragment thereof as described herein.

As used herein, by an “effective amount” is meant an amount or dosage sufficient to effect beneficial or desired results including halting, slowing, retarding, or inhibiting progression of a disease, e.g., an autoimmune disease or a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-MET, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1 (IDO1) (e.g., epacadostat).

In some embodiments, the additional therapeutic agent can comprise one or more inhibitors selected from the group consisting of an inhibitor of HER3, an inhibitor of MET, an inhibitor of heregulin, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, a HER2 agonist and a Heregulin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, anti-ICOS antibody, anti-CD27 antibody, anti-OX40 antibody, anti-4-1BB antibody, anti-CD40 antibody, anti-VEGFR2 antibody, anti-EGFR antibody, anti-HER2 antibody, and/or an anti-GITR antibody.

In one aspect, the disclosure provides a combination therapy. In some embodiments, the anti-HER3 antibody or antigen-binding fragment thereof (e.g., any antibody described herein) can be administered together with an anti-PD1 antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Human Anti-HER3 Antibodies

Human HER3 protein (hHER3-His, ACRO Biosystems Inc., Cat #: ER3-H5223) or DNA encoding this protein was emulsified with adjuvants, and was used to immunize RenLite™ mice (Biocytogen, complete human heavy chain variable domain combined with a common light chain substitution in situ). The RenLite™ mice are described, e.g., in PCT/CN2021/097652, which is incorporated herein by reference in its entirety. Before immunization, retro-orbital blood was collected as a negative control.

Freund's complete adjuvant (CFA) was used for the first immunization and Freund's incomplete adjuvant (IFA) was used for the second, third, and fourth immunizations. A total of four immunizations were performed. The first and second immunizations were separated by two weeks, and the remaining immunizations were separated by one week. One week after the fourth immunization, retro-orbital blood was collected, and the antibody titer of serum was detected by fluorescence activated cell sorting (FACS). One week later, mice with high titer were further injected with hHER3-His protein through intraperitoneal injection for impulse immunization.

Antigen-specific immune cells were isolated from the immunized mice to further obtain anti-HER3 antibodies or to obtain the light chain and heavy chain variable region sequences of the anti-HER3 antibodies. For example, single cell technology (e.g., Beacon® Optofluidic System, Berkeley Lights Inc.) was used to screen and find plasma cells secreting antigen-specific monoclonal antibodies, and then reverse transcription and PCR sequencing were used to obtain antibody variable region sequences. The obtained variable region sequences were cloned into a vector containing a sequence encoding the human IgG1 constant region for antibody expression. Binding of the expressed antibodies to HER3 was verified by FACS. Exemplary antibodies obtained by this method included: 1B2, 1C5, 1D6, 3E1 and 3G6.

These antibodies contained substantially the same light chain, and their VH and VL CDR 1-3 sequences are shown in FIG. 3A or FIG. 4A. The VH and VL regions of 1B2, 1C5, 1D6, 3E1 and 3G6 are shown in FIG. 5.

Above anti-HER3 antibodies can be paired to form various bispecific antibodies. Vectors encoding the light chain and heavy chain of the antibodies were constructed. CHO-S cells were co-transfected with three vectors, including a first vector encoding the heavy chain of an anti-HER3 antibody, a second vector encoding the heavy chain of another anti-HER3 antibody, and a third vector encoding the common light chain. After 14 days of culture, the cell supernatant was collected and purified by Protein A affinity chromatography.

Similarly, above anti-HER3 antibodies can be also used to form bispecific antibodies targeting HER3 and another target protein (e.g., MET). 2F11, 8E2, 8D9 and 8H10 are anti-MET monoclonal antibodies developed by Biocytogen Pharmaceuticals (Beijing) Co., Ltd., and these antibodies have the same light chain, whose sequence is consistent with the common light chain sequence of the anti-HER3 antibodies, and their VH CDR 1-3 sequences are shown in FIG. 3B and FIG. 4B. The VL and VH regions of 2F11 and 8E2 are shown in FIG. 5. Details related to 2F11, 8E2, 8D9 and 8H10 are described in PCT/CN2022/141416 or PCT/CN2023/103811, which are incorporated herein by reference in its entirety.

Knobs-into-holes mutations were introduced in the Fc regions of the antibodies to reduce the chance of wrong pairing between the two heavy chains. Exemplary bispecific antibodies obtained include: 3E1-1C5, 3G6-1C5, 1B2-1C5, 3E1-2F11, 3G6-2F11, 1B2-2F11, 1C5-2F11, 2F11-3E1, 2F11-3G6, 2F11-1B2, 2F11-1C5, 3E1-8E2, 3G6-8E2, 1B2-8E2, 1C5-8E2, 8E2-3E1, 8E2-3G6, 8E2-1B2, 8E2-1C5, 3E1-8D9, 1B2-8D9, 1C5-8D9, 3E1-8H10, 1B2-8H10, 1C5-8H10. In 3E1-1C5, the heavy chain constant region of 3E1 includes knob mutations, and the heavy chain constant region of 1C5 includes hole mutations. In 3G6-1C5, the heavy chain constant region of 3G6 includes knob mutations, and the heavy chain constant region of 1C5 includes hole mutations. The sequence of the human IgG1 constant region with knob mutations is set forth in SEQ ID NO: 49, and the human IgG1 constant region with hole mutations is set forth in SEQ ID NO: 50.

### Example 2. Cross-Species Binding of Anti-HER3 Antibodies

CHO-hHER3 cells, CHO-mHER3 cells, CHO-fasHER3 cells or CHO-dHER3 cells were transferred to a 96-well plate at a density of 5×10⁴ cells/well respectively. The anti-HER3 antibodies were added to the 96-well plate, and incubated at 4° C. for 30 min. Then, the cells were incubated with the secondary antibody anti-hIgG-Fc-Alex Flour 647 (RL1-H) (Jackson ImmunoResearch Laboratories, Inc., Cat #: 109-606-170) at 4° C. in the dark for 15 minutes before flow cytometry analysis.

CHO-hHER3 cells, CHO-mHER3 cells, CHO-fasHER3 cells and CHO-dHER3 cells were obtained by transfecting CHO-S cells with human HER3 (hHER3, SEQ ID NO: 43), monkey (Macaca fascicularis) HER3 (fasHER3, SEQ ID NO: 44), dog HER3 (dHER3, SEQ ID NO: 45), and mouse HER3 (mHER3, SEQ ID NO: 46), respectively.

The test results are shown in the table below. All anti-HER3 antibodies 1B2, 1C5, 1D6, 3E1 and 3G6 can bind to human HER3, monkey HER3 and dog HER3. Among these antibodies, 1B2, 1D6, 3E1 and 3G6 can also bind to mouse HER3.

TABLE 1

| Antibody | HER3 Protein | Percentage of Positive Cells | Evaluation |
|---|---|---|---|
| NC | hHER3 | 0.4% | No Binding |
| | mHER3 | 0.2% | No Binding |
| | fasHER3 | 0.2% | No Binding |
| | dHER3 | 1.0% | No Binding |
| NC + secondary | hHER3 | 1.7% | No Binding |
| antibody | mHER3 | 0.5% | No Binding |
| | fasHER3 | 0.9% | No Binding |
| | dHER3 | 1.6% | No Binding |
| 1B2 | hHER3 | 71.3% | Binding |
| | mHER3 | 22.4% | Binding |
| | fasHER3 | 69.0% | Binding |
| | dHER3 | 32.1% | Binding |
| 1C5 | hHER3 | 74.0% | Binding |
| | mHER3 | 3.4% | No Binding |
| | fasHER3 | 72.8% | Binding |
| | dHER3 | 33.1% | Binding |
| 1D6 | hHER3 | 68.0% | Binding |
| | mHER3 | 58.9% | Binding |
| | fasHER3 | 62.9% | Binding |
| | dHER3 | 38.1% | Binding |
| 3E1 | hHER3 | 70.6% | Binding |
| | mHER3 | 23.0% | Binding |
| | fasHER3 | 69.4% | Binding |
| | dHER3 | 13.2% | Binding |
| 3G6 | hHER3 | 66.5% | Binding |
| | mHER3 | 15.2% | Binding |
| | fasHER3 | 65.6% | Binding |
| | dHER3 | 29.8% | Binding |

In a similar experiment, the cross-species binding activities of anti-HER3/MET bispecific antibodies were tested. Specifically, CHO-hHER3 cells, CHO-fasHER3 cells, NIH3T3-hMET cells or NIH3T3-fasMET cells were transferred to a 96-well plate at a density of 5×10⁴ cells/well respectively. The antibodies were added to the 96-well plate, and incubated at 4° C. for 30 min. Then, the cells were incubated with the secondary antibody anti-hIgG-Fc-Alex Flour 647 (RL1-H) at 4° C. in the dark for 15 minutes before flow cytometry analysis.

NIH3T3-hMET cells and NIH3T3-fasMET cells were obtained by transfecting NIH3T3 cells with plasmids expressing human MET (hMET, SEQ ID NO: 65) and monkey MET (fasMET, SEQ ID NO: 66) respectively.

The test results are shown in the table below. All anti-HER3/MET bispecific antibodies can bind to human HER3, monkey HER3, human MET and monkey MET.

TABLE 2

| | | Positive | | | Positive | |
|---|---|---|---|---|---|---|
| Protein | Antibody | Cells | Evaluation | Antibody | Cells | Evaluation |
| hHER3 | NC | 2.1% | No Binding | NC + | 2.8% | No Binding |
| fasHER3 | | 3.2% | No Binding | secondary | 2.7% | No Binding |
| hMET | | 3.9% | No Binding | antibody | 2.4% | No Binding |
| fasMET | | 0.5% | No Binding | | 0.6% | No Binding |
| hHER3 | 1B2-2F11 | 51.4% | Binding | 3G6-8E2 | 55.2% | Binding |
| fasHER3 | | 56.3% | Binding | | 58.4% | Binding |
| hMET | | 99.8% | Binding | | 99.6% | Binding |
| fasMET | | 66.4% | Binding | | 51.6% | Binding |
| hHER3 | 1C5-2F11 | 56.0% | Binding | 3E1-8D9 | 59.0% | Binding |
| fasHER3 | | 54.9% | Binding | | 58.6% | Binding |
| hMET | | 99.7% | Binding | | 99.9% | Binding |
| fasMET | | 68.4% | Binding | | 70.2% | Binding |
| hHER3 | 3E1-2F11 | 56.4% | Binding | 1B2-8D9 | 54.0% | Binding |
| fasHER3 | | 50.3% | Binding | | 55.5% | Binding |
| hMET | | 99.8% | Binding | | 99.8% | Binding |
| fasMET | | 87.3% | Binding | | 64.9% | Binding |
| hHER3 | 3G6-2F11 | 47.3% | Binding | 1C5-8D9 | 64.8% | Binding |
| fasHER3 | | 56.0% | Binding | | 60.4% | Binding |
| hMET | | 99.8% | Binding | | 99.8% | Binding |
| fasMET | | 72.9% | Binding | | 65.1% | Binding |
| hHER3 | 1B2-8E2 | 56.3% | Binding | 3E1-8H10 | 72.1% | Binding |
| fasHER3 | | 61.4% | Binding | | 66.0% | Binding |
| hMET | | 99.6% | Binding | | 99.7% | Binding |
| fasMET | | 54.3% | Binding | | 54.6% | Binding |
| hHER3 | 1C5-8E2 | 56.9% | Binding | 1B2-8H10 | 67.9% | Binding |
| fasHER3 | | 55.1% | Binding | | 64.4% | Binding |
| hMET | | 99.6% | Binding | | 99.8% | Binding |
| fasMET | | 55.1% | Binding | | 57.2% | Binding |
| hHER3 | 3E1-8E2 | 58.4% | Binding | 1C5-8H10 | 67.3% | Binding |
| fasHER3 | | 63.9% | Binding | | 58.9% | Binding |
| hMET | | 99.5% | Binding | | 99.8% | Binding |
| fasMET | | 54.8% | Binding | | 54.8% | Binding |

### Example 3. Cross Binding Activity of Anti-HER3 Antibody to the HER Family Proteins

The cross binding activities of anti-HER3 antibodies to HER family proteins were determined by surface plasmon resonance (SPR) using Biacore (Biacore, INC, Piscataway N.J.) 8K biosensor equipped with pre-immobilized Protein A sensor chips.

The HER family proteins include human EGFR (hEGFR, ACRO Biosystems Inc., Cat #: EGR-H5222), human HER2 (hHER2, ACRO Biosystems Inc., Cat #: HE2-H5225), human HER3 (hHER3, ACRO Biosystems Inc., Cat #: ER3-H5223) and human HER4 (hHER4, ACRO Biosystems Inc., Cat #: ER4-H5221).

Purified anti-HER3 antibodies were diluted to 2 μg/mL and then injected into the Biacore 8K biosensor at 10 μL/min for about 50 seconds to achieve a desired protein density (e.g., about 200 response units (RU)). Anti-HER3 antibody patritumab analog was used as a positive control. Kinetic measurements were performed at the concentrations 0 nM, 3.125 nM, 6.25 nM, 12.5 nM, 25 nM, 50 nM, 100 nM, 200 nM, and 400 nM of the recombinant His-tagged HER family protein. The association phase lasted for 180 s and the dissociation phase lasted 400 s followed by a regeneration step with Glycine (pH 2.0, 30 μL/min for 30 seconds). For isotype control (ISO), an antibody targeting an irrelevant target protein was used.

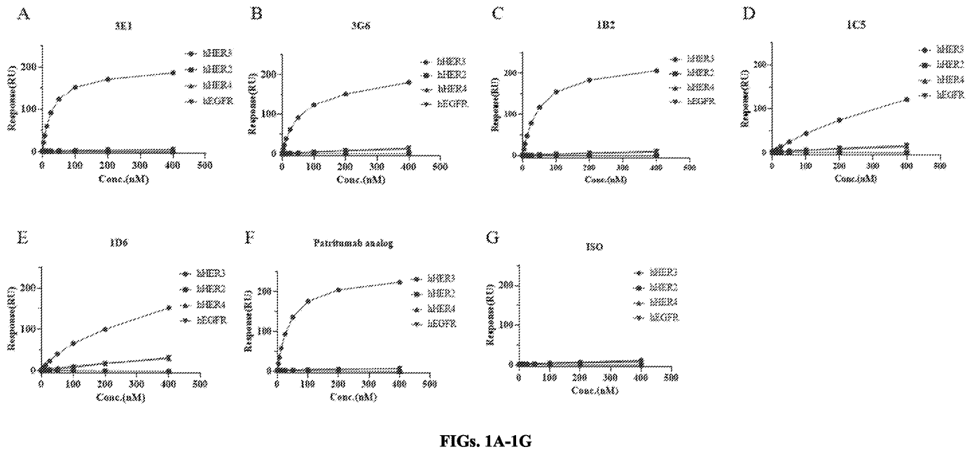

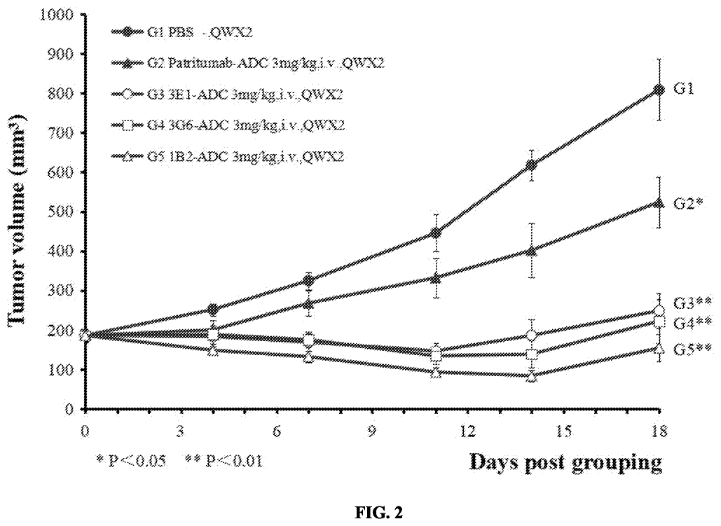

The results are shown in FIGS. 1A-1G. Anti-HER3 antibodies 1B2, 1C5, 3E1 and 3G6 don't bind to other human HER family proteins except HER3.

Patritumab is an anti-HER3 fully human monoclonal antibody which had been developed at Daiichi sankyo, and its heavy chain variable region and light chain variable region are shown in SEQ ID NO: 47 and SEQ ID NO: 48, respectively.

### Example 4. Internalization of Anti-HER3 Antibodies

Anti-HER3 antibodies (2.5 μg/mL) and pHAb-AffiniPure Fab Goat Anti-Human IgG Secondary Antibody were added to the NUGC-4 cells (Cobioer, Cat #: CBP60493), MCF-7 cells (ATCC, Cat #: CRL-3435) or LN229 cells (ATCC, Cat #: CRL-2611) and incubated for 4-6 hours. By RNA sequence detection, the expression levels of HER3 in NUGC-4 cells, MCF-7 cells and LN229 cells were 155, 76 and 53, respectively. After incubating, the cells were centrifuged and washed in FACS buffer. MFI was detected on a flow cytometer, and the endocytosis rates of anti-HER3 antibodies were calculated. For ISO control, human IgG1 was used. The results are summarized in the table below.

TABLE 3

| | LN229 | | NUGC-4 | | MCF-7 | |
|---|---|---|---|---|---|---|
| | | Percentage | | Percentage | | Percentage |
| | | of Positive | | of Positive | | of Positive |
| Antibody | MFI | cells | MFI | cells | MFI | cells |
| ISO | 6629 | 2.2% | 6877 | 0.57% | 8617 | 0.59% |
| 1B2 | 12466 | 7.4% | 37647 | 98.7% | 20593 | 28.0% |
| 1C5 | 13654 | 25.0% | 47249 | 99.1% | 19201 | 17.3% |
| 1D6 | 10269 | 12.8% | — | — | — | — |
| 3E1 | 14608 | 25.1% | 37460 | 98.8% | 20289 | 23.3% |
| 3G6 | 14437 | 24.7% | 36627 | 98.1% | 19161 | 18.8% |
| 3E1-1C5 | — | — | 82036 | 99.8% | 23937 | 51.3% |
| 1B2-1C5 | — | — | 67017 | 99.5% | 25890 | 62.0% |
| 3G6-1C5 | — | — | 64072 | 99.6% | 24974 | 58.5% |

— No detect.

The data showed that, 1B2, 1C5, 1D6, 3E1 and 3G6 exhibited good endocytosis rates in LN229 cells, NUGC-4 cells and MCF-7 cells. In addition, the endocytosis activities of the bispecific antibodies 3E1-1C5, 1B2-1C5 and 3G6-1C5 were significantly enhanced compared with the monoclonal antibodies 3E1, 1B2, 3G6 and 1C5 in NUGC-4 cells and MCF-7 cells.

In a similar experiment, anti-HER3 antibodies (2.5 μg/mL) and pHAb-AffiniPure Fab Goat Anti-Human IgG Secondary Antibody were added to dog breast cancer CMT-U27 cells (ATCC, Cat #: CRL-3456). After incubating for 6 hours, the cells were centrifuged and washed in FACS buffer. MFI was detected on a flow cytometer, and the endocytosis rates of anti-HER3 antibodies were calculated. The results showed that the percentage of positive cells and MFI of 3E1 were 15.7% and 9796 respectively, the percentage of positive cells and MFI of the positive control Partritumab analog were 3.0% % and 8403 respectively, which indicated that 3E1 had good endocytosis rates in CMT-U27 cells.

In another similar experiment, anti-HER3/MET bispecific antibodies (2.5-10 μg/mL) and pHAb-AffiniPure Fab Goat Anti-Human IgG Secondary Antibody were added to SNU-5 cells (ATCC, Cat #: CRL-5973), Hep3B cells (ATCC, Cat #: HB-8064), HepG2 cells (ATCC, Cat #: HB-8065), PLC-PRF-5 cells (ATCC, Cat #: CRL-8024) or NUGC-4 cells. After incubating for 6 hours, the cells were centrifuged and washed in FACS buffer. MFI was detected on a flow cytometer, and the endocytosis rates of the antibodies were calculated. The results are summarized in the table below.

Telisotuzumab is a humanized IgG1 monoclonal antibody targeting MET, which is in early clinical development at AbbVie for the treatment of advanced solid tumors with MET gene amplification, and its heavy chain variable region and light chain variable region sequences are shown in SEQ ID NO: 67 and SEO ID NO: 68, respectively.

TABLE 4

| | SNU-5 | | Hep3B | | HepG2 | | PLC-PRF-5 | | NUGC-4 | |
|---|---|---|---|---|---|---|---|---|---|---|
| | Positive | | Positive | | Positive | | Positive | | Positive | |
| Antibody | cells | MFI | cells | MFI | cells | MFI | cells | MFI | cells | MFI |
| ISO | 0.8% | 13137 | 4.1% | 9273 | 1.8% | 10444 | 3.7% | 15324 | 2.2% | 7210 |
| Patritumab | 2.3% | 14388 | 8.4% | 11001 | 71.2% | 29062 | 44.3% | 41980 | 98.1% | 36717 |
| analog | | | | | | | | | | |
| Telisotuzumab | 86.9% | 85714 | 9.4% | 11551 | 20.6% | 16179 | 6.9% | 22051 | 78.3% | 19961 |
| analog | | | | | | | | | | |
| 1B2 | 1.8% | 13991 | 6.2% | 10307 | 60.5% | 26134 | 35.0% | 36882 | 96.6% | 27265 |
| 1C5 | 1.6% | 14540 | 6.2% | 10446 | 55.6% | 24743 | 40.6% | 38870 | 98.1% | 29500 |
| 3E1 | 2.9% | 15110 | 7.6% | 10419 | 61.0% | 25966 | 42.3% | 41688 | 98.1% | 30894 |
| 3G6 | 2.4% | 14338 | 7.3% | 10494 | 53.9% | 24269 | 49.0% | 43628 | 95.1% | 25089 |
| 2F11 | 84.4% | 117442 | 9.4% | 11376 | 28.7% | 16548 | 34.2% | 32975 | 79.7% | 31444 |
| 8E2 | 89.5% | 83933 | 11.6% | 12884 | — | — | — | — | 72.4% | 23930 |
| 1B2-2F11 | 96.0% | 154299 | 16.0% | 13411 | — | — | — | — | 94.7% | 60197 |
| 1C5-2F11 | 94.4% | 116623 | 13.9% | 12854 | — | — | — | — | 94.3% | 50541 |
| 3E1-2F11 | 94.7% | 70708 | 14.8% | 12753 | — | — | — | — | 94.8% | 55080 |
| 3G6-2F11 | 95.9% | 139897 | 15.1% | 13374 | — | — | — | — | 91.7% | 52926 |
| 1B2-8E2 | 80.3% | 45977 | 10.0% | 12627 | — | — | — | — | 97.4% | 40520 |
| 2F11-1B2 | — | — | — | — | 77.2% | 34689 | 54.2% | 48968 | — | — |
| 2F11-1C5 | — | — | — | — | 61.5% | 26989 | 47.7% | 43475 | — | — |
| 2F11-3E1 | — | — | — | — | 80.2% | 36995 | 67.4% | 65242 | — | — |
| 2F11-3G6 | — | — | — | — | 72.9% | 32318 | 60.6% | 55542 | — | — |

— No detect.

Anti-HER3/MET bispecific antibodies exhibited better or comparable endocytosis efficiency than that of the positive controls Patritumab analog and Telisotuzumab analog. In addition, the endocytosis efficiency of anti-HER3/MET bispecific antibodies was retained or improved as compared to that of the parental monoclonal antibodies.

### Example 5. Binding Affinity of Anti-HER3 Antibodies

The binding affinity of the anti-HER3 antibodies to His-tagged HER3 protein of human (hHER3, ACRO Biosystems Inc., Cat #: ER3-H5223), monkey (fasHER3, Sino Biological, Inc., Cat #: 90043-K08H) or dog (dHER3, SEQ ID NO: 45) were verified using Biacore™ (Biacore, Inc., Piscataway N.J.) 8K biosensor equipped with pre-immobilized Protein A sensor chips.

Purified anti-HER3 antibodies was captured on the Protein A chip (Series S Sensor Chip Protein A) for the detection. 1 μg/mL anti-HER3 antibodies was loaded at 10 μL/min to bind to a gradient concentration of hHER3, fasHER3 and dHER3 (400, 200, 100, 50, 25, 12.5, 6.25, 3.125, 1.5626 and 0 nM). For ISO control, an antibody targeting an irrelevant target protein was used. The flow rate was 30 μL/min, the binding and dissociation time were set to 180 s and 400 s, respectively. The chip was regenerated after the last injection of each titration with a glycine solution (pH 2.0) at 30 μL/min for 30 seconds.

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below.

TABLE 5

| Antibody | Protein | kon (1/Ms) | koff (1/s) | KD (M) |
|---|---|---|---|---|
| ISO | hHER3 | — | — | No binding |
| | fasHER3 | — | — | No binding |
| | dHER3 | — | — | No binding |
| 1B2 | hHER3 | 1.79E+05 | 2.39E−03 | 1.33E−08 |
| | fasHER3 | 1.00E+05 | 2.24E−03 | 2.23E−08 |
| | dHER3 | 3.61E+04 | 2.23E−03 | 6.18E−08 |
| 1C5 | hHER3 | 2.12E+04 | 6.37E−04 | 3.00E−08 |
| | fasHER3 | 1.84E+04 | 5.82E−04 | 3.17E−08 |
| | dHER3 | 1.25E+04 | 4.18E−04 | 3.34E−08 |
| 1D6 | hHER3 | 9.45E+04 | 7.69E−03 | 8.14E−08 |
| | fasHER3 | 6.80E+04 | 9.80E−04 | 1.44E−08 |
| | dHER3 | 5.81E+04 | 4.50E−03 | 7.74E−08 |
| 3E1 | hHER3 | 2.77E+05 | 3.19E−03 | 1.15E−08 |
| | fasHER3 | 1.52E+05 | 2.89E−03 | 1.91E−08 |
| | dHER3 | 5.71E+04 | 2.88E−03 | 5.04E−08 |
| 3G6 | hHER3 | 2.61E+05 | 9.10E−03 | 3.48E−08 |
| | fasHER3 | 1.64E+05 | 8.34E−03 | 5.09E−08 |
| | dHER3 | 5.55E+04 | 8.09E−03 | 1.46E−07 |

Similar experimental procedures were carried out to test the binding affinity of the anti-HER3/MET bispecific antibodies to His-tagged human HER3 (hHER3), His-tagged monkey HER3 (fasHER3), His-tagged human MET (hMET, Sino Biological, Inc., Cat #: 10692-H08H) or His-tagged monkey MET (fasMET, Sino Biological, Inc., Cat #: 90304-C08H).

Purified antibodies were captured on the Protein A chip (Series S Sensor Chip Protein A) for the detection. 2 μg/mL antibodies were loaded at 10 μL/min to bind to hHER3, fasHER3, hMET or fasMET (400 and 0 nM). The flow rate was 30 μL/min, the binding and dissociation time were set to 180 s and 400 s, respectively. The chip was regenerated after the last injection of each titration with a glycine solution (nH 2.0) at 30 μL/min for 30 seconds. The results are summarized in the table below.

TABLE 6

| | | kon | koff | KD | | kon | koff | KD |
|---|---|---|---|---|---|---|---|---|
| Protein | Ab | (1/Ms) | (1/s) | (M) | Ab | (1/Ms) | (1/s) | (M) |
| hHER3 | 1B2- | 1.57E+05 | 2.53E−03 | 1.61E−08 | 3G6- | 2.61E+05 | 1.01E−02 | 3.89E−08 |
| fasHER3 | 2F11 | 6.56E+04 | 2.55E−03 | 3.89E−08 | 8E2 | 1.52E+05 | 1.02E−02 | 6.72E−08 |
| hMET | | 6.32E+04 | 2.55E−03 | 4.04E−08 | | 7.67E+04 | 1.13E−03 | 1.48E−08 |
| fasMET | | 6.82E+04 | 4.21E−03 | 6.17E−08 | | 1.02E+06 | 9.64E−03 | 9.43E−09 |
| hHER3 | 1B2- | 1.83E+05 | 2.52E−03 | 1.38E−08 | 3E1- | 2.27E+05 | 3.41E−03 | 1.50E−08 |
| fasHER3 | 8E2 | 7.93E+04 | 2.50E−03 | 3.15E−08 | 8D9 | 9.73E+04 | 3.22E−03 | 3.31E−08 |
| hMET | | 7.14E+04 | 9.98E−04 | 1.40E−08 | | 2.34E+04 | 5.10E−04 | 2.18E−08 |
| fasMET | | 6.61E+04 | 1.13E−03 | 1.71E−08 | | 2.48E+04 | 1.03E−03 | 4.17E−08 |
| hHER3 | 1C5- | 1.87E+04 | 5.48E−04 | 2.93E−08 | 1B2- | 1.57E+05 | 2.62E−03 | 1.67E−08 |
| fasHER3 | 2F11 | 5.63E+03 | 5.82E−04 | 1.03E−07 | 8D9 | 6.71E+04 | 2.61E−03 | 3.89E−08 |
| hMET | | 6.38E+04 | 2.82E−03 | 4.42E−08 | | 2.72E+04 | 9.73E−04 | 3.57E−08 |
| fasMET | | 6.28E+04 | 4.52E−03 | 7.20E−08 | | 3.35E+04 | 1.66E−03 | 4.96E−08 |
| hHER3 | 1C5- | 2.37E+04 | 7.77E−04 | 3.27E−08 | 1C5- | 1.94E+04 | 6.05E−04 | 3.11E−08 |
| FasHER3 | 8E2 | 9.12E+03 | 8.44E−04 | 9.25E−08 | 8D9 | 6.92E+03 | 6.71E−04 | 9.70E−08 |
| hMET | | 6.80E+04 | 1.22E−03 | 1.80E−08 | | 2.36E+04 | 5.31E−04 | 2.25E−08 |
| fasMET | | 6.00E+04 | 1.58E−03 | 2.63E−08 | | 2.56E+04 | 1.06E−03 | 4.13E−08 |
| hHER3 | 3E1- | 2.31E+05 | 3.42E−03 | 1.48E−08 | 3E1- | 2.29E+05 | 3.32E−03 | 1.45E−08 |
| FasHER3 | 2F11 | 9.64E+04 | 3.25E−03 | 3.37E−08 | 8H10 | 9.87E+04 | 3.18E−03 | 3.22E−08 |
| hMET | | 6.61E+04 | 2.85E−03 | 4.31E−08 | | 4.08E+05 | 5.16E−03 | 1.27E−08 |
| fasMET | | 6.57E+04 | 4.72E−03 | 7.19E−08 | | 6.79E+05 | 2.01E−02 | 2.96E−08 |
| hHER3 | 3E1- | 2.66E+05 | 3.50E−03 | 1.32E−08 | 1B2- | 1.57E+05 | 2.65E−03 | 1.69E−08 |
| FasHER3 | 8E2 | 1.13E+05 | 3.29E−03 | 2.90E−08 | 8H10 | 6.78E+04 | 2.63E−03 | 3.87E−08 |
| hMET | | 7.11E+04 | 1.20E−03 | 1.69E−08 | | 5.74E+05 | 6.26E−03 | 1.09E−08 |
| fasMET | | 6.42E+04 | 1.50E−03 | 2.34E−08 | | 8.24E+05 | 2.28E−02 | 2.76E−08 |
| hHER3 | 3G6- | 2.24E+05 | 1.06E−02 | 4.74E−08 | 1C5- | 2.08E+04 | 5.93E−04 | 2.85E−08 |
| FasHER3 | 2F11 | 1.14E+05 | 1.03E−02 | 9.02E−08 | 8H10 | 8.04E+03 | 6.55E−04 | 8.15E−08 |
| hMET | | 5.05E+04 | 2.56E−03 | 5.07E−08 | | 3.33E+05 | 4.27E−03 | 1.28E−08 |
| fasMET | | 6.42E+04 | 4.68E−03 | 7.30E−08 | | 5.21E+05 | 1.43E−02 | 2.76E−08 |

The results show that the anti-HER3 antibodies can all bind to human, monkey and dog HER3 with high affinity. And all the anti-HER3/MET bispecific antibodies can bind to human HER3, monkey HER3, human MET and monkey MET.

### Example 6. Stability Analysis of Anti-HER3 Antibodies

The biophysical properties and stability of the anti-HER3 antibodies were evaluated. Antibody sample processing procedures are as follows:
1) The antibody was placed in a buffer solution containing 3 mg/ml histidine, 80 mg/ml sucrose, and 0.2 mg/ml Tween 80 at pH 6.0, to reach a final concentration of 2 mg/mL, and kept in sealed Eppendorf tubes at 40±2° C., 60%±5% RH (hereinafter referred to as 40° C.) for 7 days, and the thermal stability was evaluated.
2) The antibody was loaded into protein A column and eluted with a buffer (0.1 mol/L HAc) at pH 3.5. Half of the antibody was added into 2M Tris buffer to adjust the pH to 7.5 immediately. The remaining half was kept at pH 3.5 for 6 hours before adjusting the pH to 7.5. The diluted antibody was kept in sealed Eppendorf tubes at pH 3.5=0.1, 25±2° C. (hereinafter referred to as pH 3.5) for 6 h to test stability at low pH.
3) The antibody was placed in the buffer solution (3 mg/ml histidine, 80 mg/ml sucrose, and 0.2 mg/ml Tween 80) and H₂O₂ stock was added to reach a final concentration of 0.05% and 0.5% H₂O₂ (hereinafter referred to as H₂O₂ 0.05% and H₂O₂ 0.5%). Samples were kept in sealed Eppendorf tubes at 40±3° C., 60%±5% RH for 30 min to test stability under strong oxidizing conditions.
4) The antibody was placed in the buffer solution (3 mg/ml histidine, 80 mg/ml sucrose, and 0.2 mg/ml Tween 80) and NH₄HCO₃ stock was added to reach a final concentration of 0.94% NH₄HCO₃. Samples were kept in sealed Eppendorf tubes at 40±2° C., 60%=5% RH for 6 h (hereinafter referred to as NH₄HCO₃ 6 h), 24 h (hereinafter referred to as NH₄HCO₃ 24 h) to test stability under strong deamidation conditions.

The following tests were performed: (1) detecting the purity of antibodies by Size-Exclusion High Performance Liquid Chromatography (SEC-HPLC) (indicated as the percentage of the main peak area to the sum of all peak areas (Purity, %)); (2) detecting the apparent hydrophobicity of the antibodies using the Hydrophobic Interaction Chromatography-High Performance Liquid Chromatography (HIC-HPLC) method (indicated as the retention time of the main peak (HIC, min)); (3) detecting the purity changes of antibodies by capillary electrophoresis-sodium dodecyl sulphate (CE-SDS) under non-reducing (CE-SDS (NR)) conditions (indicated as the percentage of the main peak area to the sum of all peak areas (Purity, %)); (4) detecting the pI (isoelectric point) and charge variants of the antibodies by the Capillary Isoelectric Focusing (cIEF) method (indicated as the percentages of the main component, acidic component, and alkaline component).

In the SEC-HPLC experiments, the antibody samples were diluted to 1 mg/mL with purified water and an Agilent 1290 chromatograph system (connected with XBridge Protein BEH SEC column (200 Å, Waters Corporation)) was used. The following parameters were used: mobile phase: 0.1Mphosphate buffer (PB)+10% ACN, pH7.4; flow rate: 1.8 ml/min; column temperature: 25° C.; detection wavelength: 280 nm, 220 nm; injection volume: 10 μL; sample tray temperature: about 4° C.; and running time: 7 minutes.

In the HIC-HPLC experiments, an Agilent 1260 chromatograph system (connected with ProPac HIC-10 column (4.6×250 mm, Thermo Scientific)) was used, and samples were 10 times diluted using mobile phase A. The following parameters were used: mobile phase A: 0.9 M ammonium sulfate, 0.1M phosphate buffer (PB), 10% acetonitrile pH 6.5; mobile phase B: 0.1 M phosphate buffer (PB), 10% acetonitrile pH 6.5; flow rate: 0.8 ml/min; gradient: 0 min 100% A, 2 min 100% A, 32 min 100% B, 34 min 100% B, 35 min 100% A, and 45 min 100% A; column temperature: 30° C.; detection wavelength: 280 nm, 220 nm; injection volume: 10 μg; sample tray temperature: about 10° C.; and running time: 50 minutes.

In the CE-SDS (NR) experiments, Maurice CE-SDS Size Application Kit (Protein simple, Cat #: PS-MAK03-S) were used. 60 μL antibody sample (0.5 mg/mL), 2.4 μL 25× internal standard, 3 μL 250 mM Iodoacetamide (SIGMA, Cat #: 16125) were add to a microcentrifuge tube, followed by centrifugation at 3000 rpm for 1 minute and heating in a 70° C. water bath for 10 min. The samples were then cooled to room temperature followed by centrifugation at 10000 rpm for 3 minutes. Supernatant sample preparations were then transferred to a 96-well plate and tested in Maurice. The following parameters were used: injection voltage 4.6 kV, injection time 20 seconds, separation voltage 5.75 kV, and separation time 45 minutes.

In the cIEF experiments, a Maurice cIEF Method Development Kit (Protein Simple, Cat #: PS-MDK01-C) was used for sample preparation. Specifically, 8 μL, 30 μg protein sample was mixed with the following reagents in the kit: 1 μL Maurice cIEF pI Marker-7.05, 1 μL Maurice cIEF pI Marker-10.10, 35 μL 1% Methyl Cellulose Solution, 2 μL Maurice cIEF 500 mM Arginine, 1.33 μL Ampholytes (Pharmalyte pH ranges 3-10), and 6.66 μL Ampholytes (Pharmalyte pH ranges 8-10.5) water (added to make a final volume of 100 μL). On the Maurice analyzer (Protein Simple, Santa Clara, CA), Maurice cIEF Cartridges (PS-MC02-C) were used to generate imaging capillary isoelectric focusing spectra. The sample was focused for a total of 10 minutes.

Detailed results are shown in the table below. The results showed that these anti-HER3 antibodies have good stability and physical and chemical properties.

TABLE 7

| | | | | | | | cIEF | | |
|---|---|---|---|---|---|---|---|---|---|
| | | SEC | | CE−SDS (NR) | | | Acidic | Main | Alkali |
| | | Purity | HIC | Purity | LMW | HMW | peak | peak | peak |
| Ab | Treatment | (%) | (min) | (%) | (%) | (%) | (%) | (%) | (%) |
| 1B2 | 0 d | 96.58 | 3.90 | 93.3 | 6.7 | 0 | 38.5 | 61.5 | 0 |
| | 40° C. 7 d | 96.06 | 3.92 | 93.5 | 6.5 | 0 | 48.0 | 52.0 | 0 |
| | pH3.5 0 h | 100 | 3.95 | 94.4 | 5.5 | 0 | 37.8 | 62.2 | 0 |
| | pH3.5 6 h | 100 | 3.96 | 93.2 | 6.9 | 0 | 34.5 | 65.5 | 0 |
| | H₂O₂ 0.05% | 100 | 3.42 | 90.9 | 9.0 | 0 | 37.6 | 62.4 | 0 |
| | H₂O₂ 0.5% | 100 | 2.79 | 92.3 | 7.8 | 0 | 38.2 | 61.8 | 0 |
| | NH₄HCO₃ 6 h | 100 | 3.93 | 95.3 | 4.7 | 0 | 44.3 | 55.7 | 0 |
| | NH₄HCO₃ 24 h | 100 | 3.92 | 91.9 | 8.2 | 0 | 58.9 | 41.1 | 0 |
| 1C5 | 0 d | 96.57 | 5.13 | — | — | — | 0 | 100 | 0 |
| | 40° C. 7 d | 96.36 | 5.14 | — | — | — | 0 | 100 | 0 |
| | pH3.5 0 h | 100 | 5.11 | — | — | — | 0 | 100 | 0 |
| | pH3.5 6 h | 100 | 5.12 | — | — | — | 0 | 100 | 0 |
| | H₂O₂ 0.05% | 100 | 4.50 | — | — | — | 0 | 100 | 0 |
| | H₂O₂ 0.5% | 100 | 4.09 | — | — | — | 0 | 100 | 0 |
| | NH₄HCO₃ 6 h | 100 | 5.12 | — | — | — | 0 | 100 | 0 |
| | NH₄HCO₃ 24 h | 100 | 5.13 | — | — | — | 0 | 100 | 0 |
| 1D6 | 0 d | 97.01 | 5.61 | — | — | — | 38.3 | 58.0 | 3.8 |
| | 40° C. 7 d | 93.92 | 5.57 | — | — | — | 46.9 | 49.3 | 3.8 |
| | pH3.5 0 h | 100 | 5.64 | — | — | — | 33.3 | 60.3 | 6.3 |
| | pH3.5 6 h | 100 | 5.63 | — | — | — | 54.0 | 42.1 | 3.9 |
| | H₂O₂ 0.05% | 100 | 5.28 | — | — | — | 38.0 | 57.8 | 4.2 |
| | H₂O₂ 0.5% | 100 | 4.50 | — | — | — | 40.0 | 54.5 | 4.6 |
| | NH₄HCO₃ 6 h | 100 | 5.60 | — | — | — | 41.1 | 53.6 | 4.1 |
| | NH₄HCO₃ 24 h | 100 | 5.60 | — | — | — | 33.3 | 60.5 | 6.2 |
| 3E1 | 0 d | 95.98 | 2.53 | 95.3 | 4.7 | 0 | 16.4 | 79.4 | 4.2 |
| | 40° C. 7 d | 95.98 | 2.53 | 95.8 | 4.2 | 0 | 32.5 | 53.9 | 13.6 |
| | pH3.5 0 h | 100 | 2.60 | 89.1 | 10.8 | 0 | 16.6 | 83.4 | 0 |
| | pH3.5 6 h | 100 | 2.60 | 93.5 | 6.4 | 0 | 16.8 | 83.2 | 0 |
| | H₂O₂ 0.05% | 100 | 2.28 | 94.9 | 5.0 | 0 | 18.0 | 82.0 | 0 |
| | H₂O₂ 0.5% | 100 | 1.59 | 94.0 | 6.1 | 0 | 19.2 | 80.8 | 0 |
| | NH₄HCO₃ 6 h | 100 | 2.54 | 95.6 | 4.5 | 0 | 18.5 | 81.5 | 0 |
| | NH₄HCO₃ 24 h | 100 | 2.56 | 90.1 | 9.9 | 0 | 35.2 | 64.8 | 0 |
| 3G6 | 0 d | 96.08 | 2.88 | 98.3 | 1.7 | 0 | 30.6 | 69.4 | 0 |
| | 40° C. 7 d | 96.38 | 2.87 | 97.9 | 2.1 | 0 | 35.5 | 64.5 | 0 |
| | pH3.5 0 h | 100 | 2.90 | 94.6 | 5.4 | 0 | 26.9 | 73.1 | 0 |
| | pH3.5 6 h | 100 | 2.88 | 94.0 | 6.0 | 0 | 26.6 | 73.4 | 0 |
| | H₂O₂ 0.05% | 100 | 2.48 | 96.5 | 3.5 | 0 | 0 | 100 | 0 |
| | H₂O₂ 0.5% | 100 | 2.18 | 94.5 | 5.5 | 0 | 0 | 100 | 0 |
| | NH₄HCO₃ 6 h | 100 | 2.89 | 96.9 | 3.1 | 0 | 30.1 | 69.9 | 0 |
| | NH₄HCO₃ 24 h | 100 | 2.87 | 94.3 | 5.7 | 0 | 38.5 | 61.1 | 0 |

— No Detect.

### Example 7. Generating Anti-HER3 Antibody Drug Conjugates (ADC)

Each purified antibody (3E1, 3G6, 1B2,1C5, 3E1-1C5, 3G6-1C5, 1B2-1C5, 1B2-8E2, 2F11-1B2, 2F11-1C5, 2F11-3E1 or 2F11-3G6) was coupled with MMAE (monomethyl auristatin E) or MMAF (monomethyl auristatin F) through a maleimidocaproyl-valine-citrulline-p-aminobenzyloxycarbonyl (VC) linker.

For the names of antibody drug conjugates, “ADC” is added directly after the antibody name. For example, if 3E1 is coupled to MMAE, it is named as 3E1-ADC. If 3E1-1C5 is coupled to MMAE, it is named as 3E1-1C5-ADC.

Patritumab analog and Telisotuzumab analog were also coupled with MMAE respectively for the positive controls. Human IgG1 coupling with MMAE was used for a ISO control (referring to ISO-ADC) in the following experiments.

HIC-HPLC was performed to detect the coupling of antibodies with drug molecules. The results showed that the drug-to-antibody ratio (DAR) of ADC was about 4.

### Example 8. Anti-Tumor Activity of Anti-HER3 ADC in NUGC-4 Model

B-NDG mice (Biocytogen Pharmaceuticals (Beijing) Co., Ltd., Cat #: B-CM-002) were used to determine the anti-tumor activity of anti-HER3 ADC. About 5×10⁶ stomach cancer NUGC-4 cells were injected subcutaneously in B-NDG mice, and when the tumor volume grew to about 200 mm³, the mice were divided to a control group and different treatment groups based on tumor size (5 mice per group). The treatment groups were randomly injected with patritumab-ADC, 3E1-ADC, 3G6-ADC or 1B2-ADC. The control group mice were injected with phosphate buffer saline (PBS). Patritumab analog was coupled with MMAE as a positive control (named Patritumab-ADC). The frequency of administration was once a week (2 times of administrations in total).

The tumor volume was measured twice a week and the body weight of the mice was weighed as well. Euthanasia was performed when the tumor volume of the mouse reached 3000 mm³. Details of the administration scheme are shown in the table below.

TABLE 8

| | No. of | | Dosage | | | Total No. of |
|---|---|---|---|---|---|---|
| Group | mice | Antibodies | (mg/kg) | Route | Frequency | administration |
| G1 | 5 | PBS | — | i.v. | QW | 2 |
| G2 | 5 | Patritumab-ADC | 3 mg/kg | i.v. | QW | 2 |
| G3 | 5 | 3E1-ADC | 3 mg/kg | i.v. | QW | 2 |
| G4 | 5 | 3G6-ADC | 3 mg/kg | i.v. | QW | 2 |
| G5 | 5 | 1B2-ADC | 3 mg/kg | i.v. | QW | 2 |

The length of the long axis and the short axis of the tumor were measured and the volume of the tumor was calculated as 0.5×(long axis)×(short axis) 2. The tumor growth inhibition percentage (TGI %) was calculated using the following formula: TGI (%)=[1−(Ti−T0)/(Vi−V0)]×100%. Ti is the average tumor volume in the treatment group on day i. TO is the average tumor volume in the treatment group on day zero. Vi is the average tumor volume in the control group on day i. V0 is the average tumor volume in the control group on day zero. T-test was performed for statistical analysis. A TGI % higher than 60% indicates clear suppression of tumor growth. P<0.05 is a threshold to indicate significant difference.

At the end of the experiment, there was no significant difference between each group, and all mice survived at the end of the experiment, which showed that the tested ADC were well tolerated and were not obviously toxic to the mice.

The tumor size data in groups treated with the antibodies are shown in FIG. 2. The table below summarizes the results for this experiment, including the tumor volumes on the day of grouping (day 0), 11 days after grouping (day 11) and 18 days after grouping (day 18); the survival rate of the mice; Tumor Growth Inhibition value (TGI); and the statistical differences (P value) of tumor volume and body weight between the treatment and control groups.

TABLE 9

| | | | | | | P value (Day 18) | |
|---|---|---|---|---|---|---|---|
| | Tumor volume (mm³) | | | | TGI % | Tumor | Body |
| Group | Day 0 | Day 11 | Day 18 | Survival | (Day 18) | volume | weight |
| G1 | 186 ± 10 | 445 ± 47 | 809 ± 78 | 5/5 | NA | NA | NA |
| G2 | 185 ± 14 | 332 ± 50 | 523 ± 65 | 5/5 | 45.8 | 0.023 | 0.781 |
| G3 | 186 ± 14 | 146 ± 20 | 248 ± 44 | 5/5 | 89.9 | 2.37E−04 | 0.054 |
| G4 | 185 ± 17 | 134 ± 21 | 222 ± 55 | 5/5 | 94.1 | 2.69E−04 | 0.124 |
| G5 | 185 ± 17 | 93 ± 11 | 154 ± 35 | 5/5 | 105.1 | 5.73E−05 | 0.137 |

As shown in FIG. 2 and the table above, compared with the control group (G1), the tumor growth in the treatment groups (G2 to G5) were inhibited to different extents. Among the treatment groups, 3E1-ADC treatment group (G3), 3G6 ADC treatment group (G4) and 1B2-ADC treatment group (G5) obtained better tumor inhibitory effect as compared to that of positive control group (G2). The mouse tumor volume and survival rate after Day 18 were continued to be monitored. Euthanasia was performed when the tumor volume of the mouse reached 3000 mm³. At the end of the experiment (Day 32), 3E1-ADC, 3G6-ADC and 1B2-ADC still showed stronger anti-tumor effect (with a TGI % of 42.4%, 53.4% or 64.0% respectively) than that of the positive control Patritumab-ADC (with a TGI % of −0.4%).

In a similar experiment, about 5×10⁶ stomach cancer NUGC-4 cells were injected subcutaneously in B-NDG mice, and when the tumor volume grew to about 200 mm³, the mice were divided to different groups based on tumor size (6 mice per group). The treatment groups were randomly injected with 3 mg/kg Patritumab-ADC (G3), 3G6-1C5-ADC (G4), 1B2-1C5-ADC (G5), 3G6-ADC (G6), 1B2-ADC (G7) or 1C5-ADC (G8). The control group mice were injected with PBS (G1). Human IgG1 coupling with MMAE was used as ISO control (ISO-ADC, G2). The frequency of administration was once a week (2 administrations in total). The body weight and tumor volume were measured twice a week. Euthanasia was performed when the tumor volume of the mouse reached 3000 mm³.

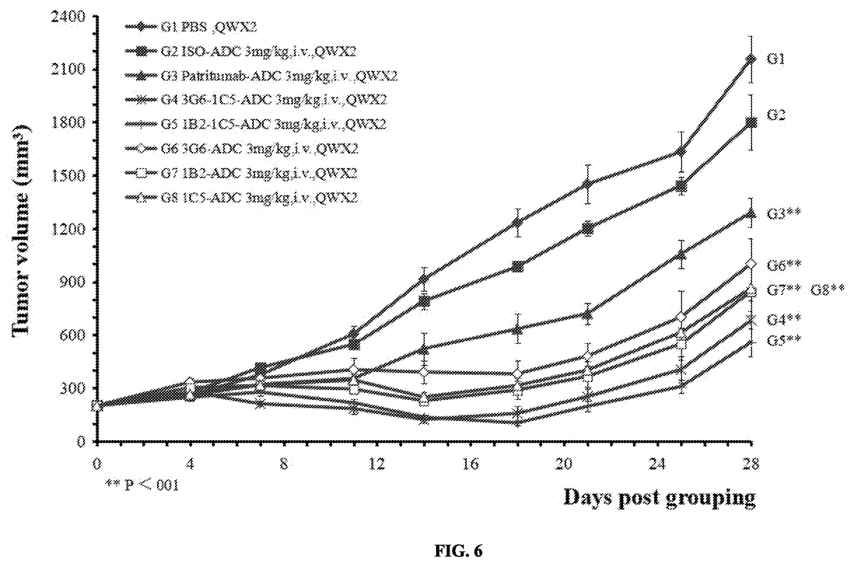

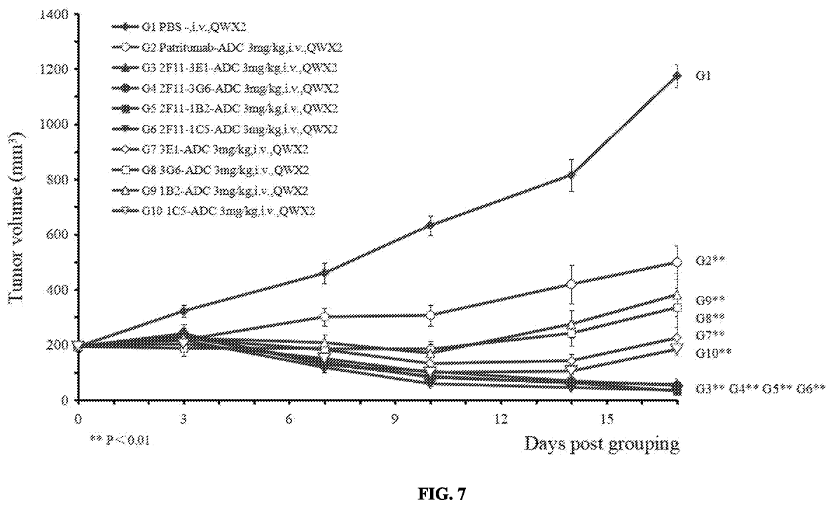

The tumor size data in groups treated with the antibodies are shown in FIG. 6. The table below summarizes the results for this experiment, including the tumor volumes on Day 0, Day 18 and Day 28; the survival rate of the mice on Day28; TGI %; and P value of tumor volume between the treatment and control groups.

TABLE 10

| | Tumor volume (mm³) | | | | TGI % | P value |
|---|---|---|---|---|---|---|
| Group | Day 0 | Day 18 | Day 28 | Survival | (Day 28) | (Day 28) |
| G1 | 200 ± 7 | 1234 ± 78 | 2156 ± 131 | 6/6 | NA | NA |
| G2 | 200 ± 10 | 986 ± 18 | 1799 ± 156 | 6/6 | 18.2 | 0.110 |
| G3 | 200 ± 10 | 635 ± 84 | 1291 ± 82 | 6/6 | 44.2 | 2.29E−04 |
| G4 | 200 ± 11 | 156 ± 42 | 682 ± 112 | 6/6 | 75.4 | 6.46E−06 |
| G5 | 200 ± 12 | 103 ± 16 | 557 ± 80 | 6/6 | 81.8 | 1.09E−06 |
| G6 | 200 ± 9 | 379 ± 74 | 1001 ± 143 | 6/6 | 59.1 | 1.37E−04 |
| G7 | 200 ± 9 | 288 ± 51 | 846 ± 136 | 6/6 | 67.0 | 3.95E−05 |
| G8 | 200 ± 10 | 314 ± 42 | 864 ± 130 | 6/6 | 66.1 | 3.66E−05 |

As shown in FIG. 6 and the table above, compared with the control group (G1), the tumor growth in the treatment groups (G2 to G8) were inhibited to different extents. Among the treatment groups, the bispecific ADCs (G4 and G5) obtained better tumor inhibitory effect as compared to that of the monoclonal antibody ADCs (G6-G8), and the monoclonal antibody ADCs showed better anti-tumor activities than that of the positive control group (G3).

In another similar experiment, about 5×10⁶ NUGC-4 cells were injected subcutaneously in B-NDG mice, and when the tumor volume grew to about 200 mm³, the mice were divided to different groups based on tumor size (5 mice per group). The treatment groups were randomly injected with ADCs. The control group mice were injected with PBS. Details of the administration scheme are shown in the table below.

TABLE 11

| | No. of | | Dosage | | | Total No. of |
|---|---|---|---|---|---|---|
| Group | mice | Antibodies | (mg/kg) | Route | Frequency | administration |
| G1 | 5 | PBS | — | i.v. | QW | 2 |
| G2 | 5 | Patritumab-ADC | 3 mg/kg | i.v. | QW | 2 |
| G3 | 5 | 2F11-3E1-ADC | 3 mg/kg | i.v. | QW | 2 |
| G4 | 5 | 2F11-3G6-ADC | 3 mg/kg | i.v. | QW | 2 |
| G5 | 5 | 2F11-1B2-ADC | 3 mg/kg | i.v. | QW | 2 |
| G6 | 5 | 2F11-1C5-ADC | 3 mg/kg | i.v. | QW | 2 |
| G7 | 5 | 3E1-ADC | 3 mg/kg | i.v. | QW | 2 |
| G8 | 5 | 3G6-ADC | 3 mg/kg | i.v. | QW | 2 |
| G9 | 5 | 1B2-ADC | 3 mg/kg | i.v. | QW | 2 |
| G10 | 5 | 1C5-ADC | 3 mg/kg | i.v. | QW | 2 |

The tumor size data in groups treated with the antibodies are shown in FIG. 7. The table below summarizes the results for this experiment, including the tumor volumes on Day 0, Day 7 and Day 17; TGI %; and P value of tumor volume between the treatment and control groups.

TABLE 12

| | Tumor volume (mm³) | | | TGI % | P value |
|---|---|---|---|---|---|
| Group | Day 0 | Day 7 | Day 17 | (Day 17) | (Day 17) |
| G1 | 193 ± 10 | 459 ± 27 | 1175 ± 42 | NA | NA |
| G2 | 193 ± 12 | 301 ± 31 | 498 ± 59 | 68.9 | 1.46E−05 |
| G3 | 193 ± 15 | 131 ± 6 | 57 ± 19 | 113.9 | 9.34E−09 |
| G4 | 193 ± 18 | 127 ± 11 | 52 ± 5 | 114.3 | 6.59E−08 |
| G5 | 193 ± 16 | 139 ± 14 | 32 ± 5 | 116.4 | 3.88E−09 |
| G6 | 193 ± 15 | 117 ± 18 | 36 ± 4 | 116.1 | 3.94E−09 |
| G7 | 193 ± 14 | 182 ± 23 | 225 ± 39 | 96.8 | 1.84E−07 |
| G8 | 193 ± 19 | 185 ± 22 | 335 ± 71 | 85.6 | 7.70E−06 |
| G9 | 193 ± 18 | 208 ± 29 | 381 ± 78 | 80.9 | 1.90E−05 |
| G10 | 193 ± 20 | 150 ± 5 | 184 ± 20 | 101.0 | 2.51E−08 |

As shown in FIG. 7 and the table above, compared with the control group (G1), the tumor growth in the treatment groups (G2 to G10) were inhibited to different extents. Among the treatment groups, the bispecific ADCs (G3-G6) obtained better tumor inhibitory effects as compared to that of the monoclonal antibody ADCs (G7-G10), and the monoclonal antibody ADCs showed better anti-tumor activities than that of the positive control group (G2).

### Example 9. Anti-Tumor Activity of Anti-HER3 ADC in Stomach PDX Model

The anti-HER3 ADC were tested for their effect in a human stomach PDX model. B-NDG mice were engrafted in the right flank with patient-derived stomach cancer fragments (2 mm×2 mm×2 mm). When the tumors in the mice reached a volume of about 250-300 mm³, the mice were randomly placed into different groups based on the volume of the tumor. The mice were then injected with PBS, patritumab-ADC, 3E1-ADC or 3G6-ADC by intravenosus (i.v.) administration. Details of the administration scheme are shown in the table below.

TABLE 13

| | No. of | | Dosage | | | Total No. of |
|---|---|---|---|---|---|---|
| Group | mice | Antibodies | (mg/kg) | Route | Frequency | administration |
| G1 | 6 | PBS | — | i.v. | QW | 2 |
| G2 | 6 | ISO | 3 mg/kg | i.v. | QW | 2 |
| G3 | 6 | Patritumab-ADC | 3 mg/kg | i.v. | QW | 2 |
| G4 | 6 | 3E1-ADC | 3 mg/kg | i.v. | QW | 2 |
| G5 | 6 | 3G6-ADC | 3 mg/kg | i.v. | QW | 2 |

The table below summarizes the results for this experiment, including the tumor volumes on the day of grouping (day 0), 9 days after grouping (day 9), 20 days after grouping (day 20) and at the end of the experiment (day 34); the survival rate of the mice; Tumor Growth Inhibition value (TGI); and the statistical differences (P value) of tumor volume between the treatment and control groups.

TABLE 14

| | Tumor volume (mm³) | | | | | TGI % | P value |
|---|---|---|---|---|---|---|---|
| Group | Day 0 | Day 9 | Day 20 | Day 34 | Survival | (Day 20) | (Day 20) |
| G1 | 242 ± 11 | 538 ± 45 | 772 ± 59 | 1312 ± 91 | 6/6 | NA | NA |
| G2 | 242 ± 19 | 445 ± 18 | 688 ± 60 | 1041 ± 105 | 6/6 | 16.0 | 0.339 |
| G3 | 243 ± 16 | 452 ± 31 | 673 ± 70 | 1201 ± 154 | 6/6 | 18.9 | 0.299 |
| G4 | 242 ± 22 | 419 ± 63 | 480 ± 71 | 951 ± 157 | 6/6 | 55.2 | 0.010 |
| G5 | 243 ± 25 | 400 ± 47 | 394 ± 53 | 999 ± 211 | 6/6 | 71.4 | 0.001 |

As shown in the table above, 3E1-ADC (G4) and 3G6-ADC (G5) inhibit tumor growth with a higher TGI % (55.2% and 71.4%) than that of positive control Patritumab-ADC (18.9%).

In a similar experiment, the anti-tumor activities of 2F11-1C5-ADC and 2F11-3G6-ADC were tested in vivo in a gastric cancer PDX model with HER and MET-low/moderate-expression. Specifically, patient-derived gastric tumor fragments (2 mm×2 mm×2 mm) were engrafted in the right flank of B-NDG mice. When the tumors in the mice reached a volume of about 250 mm³, the mice were randomly placed into different groups based on tumor volumes (5 mice per group), and then injected with PBS, ISO-ADC, Patritumab-ADC, Telisotuzumab-ADC, 2F11-1C5-ADC or 2F11-3G6-ADC by i.v. administration. The tumor volumes were measured twice a week.

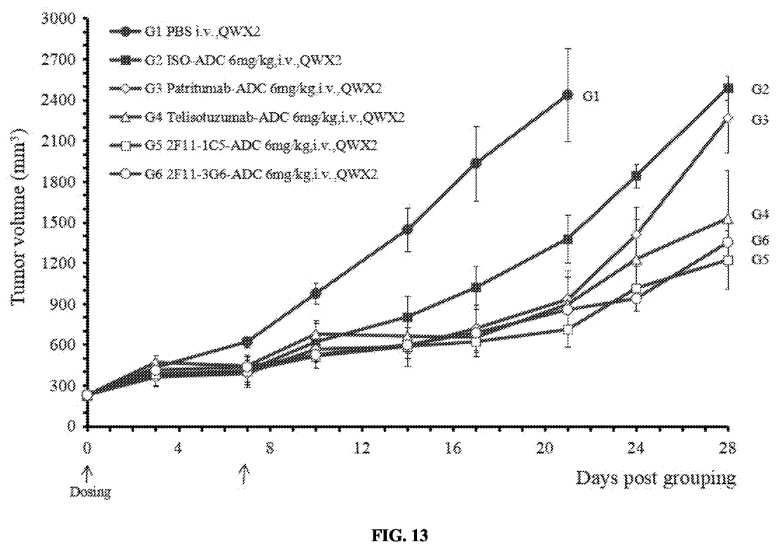

As shown in FIG. 13, the tumor volumes in all treatment groups (G2-G6) were smaller than those in the controls group (G1 and G2). The results also showed that 2F11-1C5-ADC (G5) and 2F11-3G6-ADC (G6) exhibited better anti-tumor activities than that of the positive controls Patritumab-ADC (G3) and Telisotuzumab-ADC (G4).

### Example 10. Anti-Tumor Activity of Anti-HER3 ADC in HCC70 Model

In another similar experiment, about 1×10⁷ breast cancer HCC70 cells were injected subcutaneously in B-NDG mice, and when the tumor volume grew to about 200 mm³, the mice were divided to a control group and different treatment groups based on tumor size (5 mice per group). The treatment groups were randomly selected for Patritumab-ADC treatment, 3E1-ADC treatment or 1B2-ADC treatment with 3 mg/kg. The control group mice were injected with PBS. Human IgG1 coupling with MMAE was used as ISO control. The frequency of administration was once a week (2 administrations in total).

The result showed that at the same dosage and frequency of administration, 3E1-ADC and 1B2-ADC showed better tumor inhibition efficacy (with a TGI % of 51.0% and 56.6% respectively on Day 18) than the positive control Patritumab-ADC (with a TGI % of 14.6% on Day 18).

### Example 11. Anti-Tumor Activity of Anti-HER3 ADC in Pancreatic PDX Model

The anti-HER3 ADCs were tested for their antitumor effects in a human pancreatic cancer PDX model. B-NDG mice were engrafted in the right flank with patient-derived pancreatic cancer fragments (2 mm×2 mm×2 mm). When the tumors in the mice reached a volume of about 250-300 mm³, the mice were randomly placed into different groups based on tumor volume. The mice were then injected with PBS (G1), 3 mg/kg Patritumab-ADC (G2), 3 mg/kg 3E1-ADC (G3), 3G6-ADC (G4) or 3 mg/kg 1B2-ADC (G5) by i.v. administration. The frequency of administration was once a week (2 administrations in total).

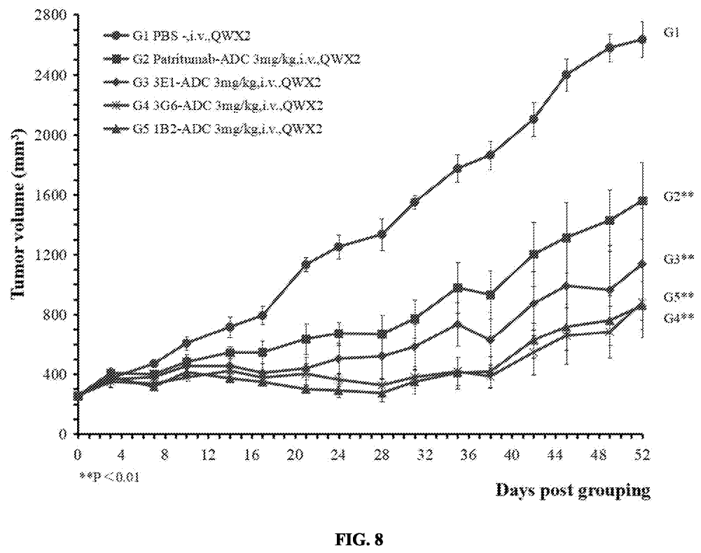

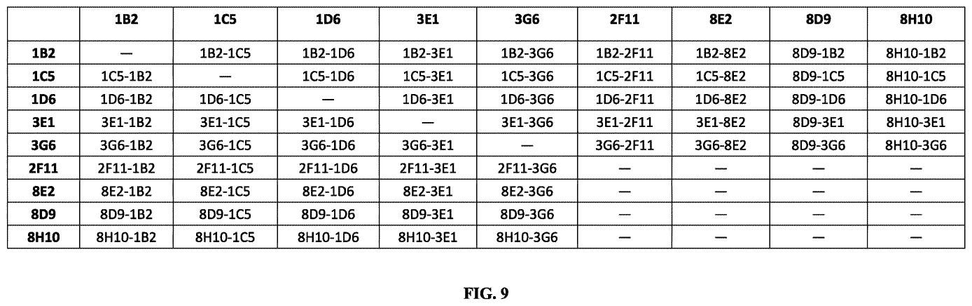

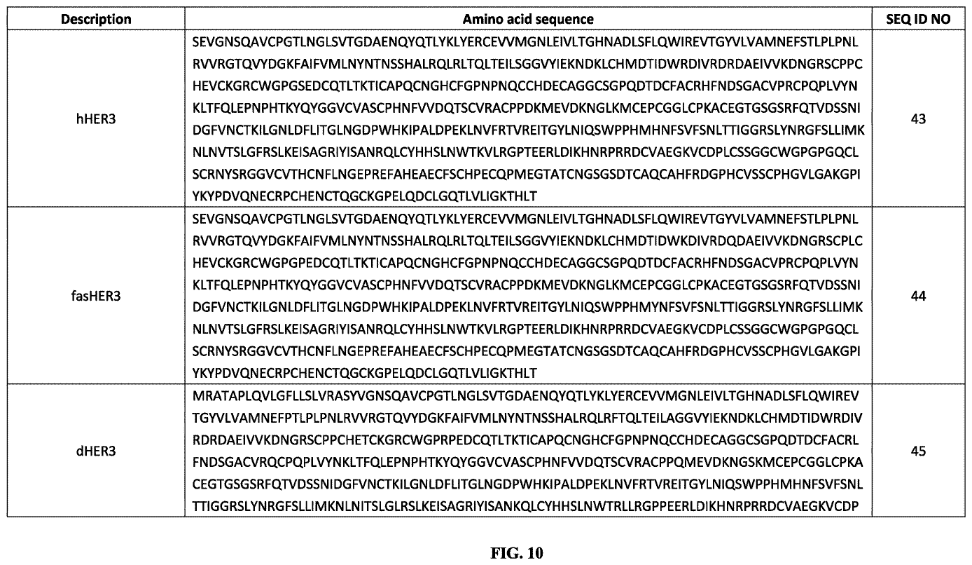

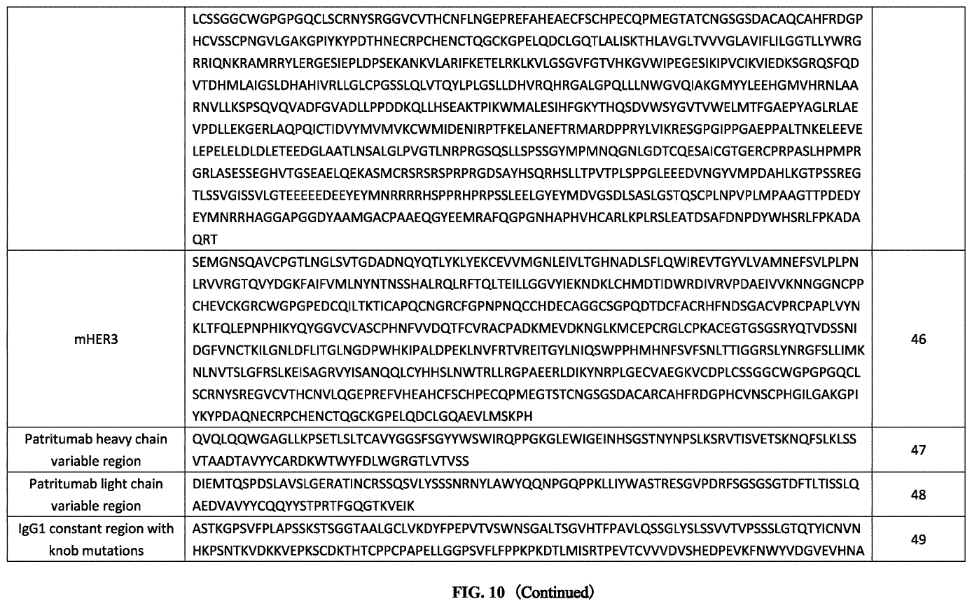

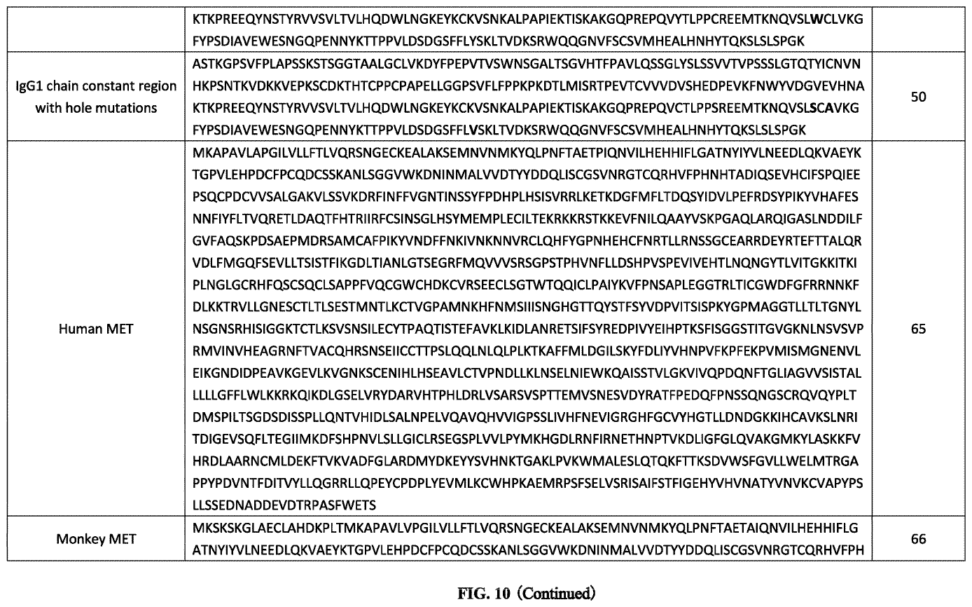

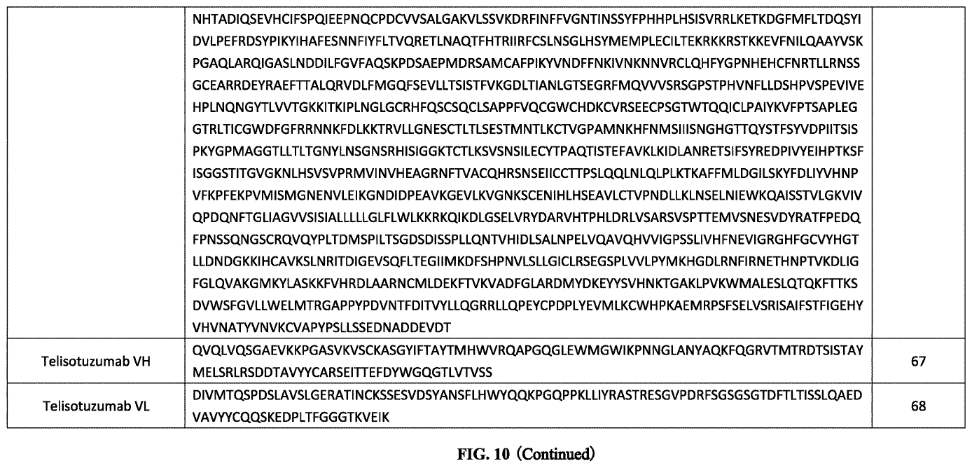

The tumor volumes of mice in different groups are shown in FIG. 8. The treatment groups (G2-G5) showed extremely significant tumor inhibition compared with PBS group (G1). In addition, 3E1-ADC (G3, TGI %-62.9%), 3G6-ADC (G4, TGI %=64.8%) and 1B2-ADC (G5, TGI %=74.6%) showed better tumor inhibition effects than that of the positive control Patritumab-ADC (G2, TGI %=45.2%) in pancreatic cancer model.

In a similar experiment, B-NDG mice were engrafted with patient-derived pancreatic cancer fragments. When the tumors reached a volume of about 250-300 mm³, the mice were randomly placed into different groups based on tumor volume. The mice were then injected with PBS, anti-HER3 ADCs or anti-HER3/MET ADCs. Details of the administration scheme are shown in the table below.

TABLE 15

| | No. of | | Dosage | | | Total No. of |
|---|---|---|---|---|---|---|
| Group | mice | ADCs | (mg/kg) | Route | Frequency | administration |
| G1 | 5 | PBS | — | i.v. | QW | 2 |
| G2 | 5 | 2F11-3E1-ADC | 3 mg/kg | i.v. | QW | 2 |
| G3 | 5 | 2F11-3G6-ADC | 3 mg/kg | i.v. | QW | 2 |
| G4 | 5 | 2F11-1B2-ADC | 3 mg/kg | i.v. | QW | 2 |
| G5 | 5 | 2F11-1C5-ADC | 3 mg/kg | i.v. | QW | 2 |
| G6 | 5 | 3E1-ADC | 3 mg/kg | i.v. | QW | 2 |
| G7 | 5 | 3G6-ADC | 3 mg/kg | i.v. | QW | 2 |
| G8 | 5 | 1B2-ADC | 3 mg/kg | i.v. | QW | 2 |
| G9 | 5 | 1C5-ADC | 3 mg/kg | i.v. | QW | 2 |

The table below summarizes the results for this experiment, including the tumor volumes on the day of grouping (Day 0), 10 days after grouping (Day 10) and 17 days after grouping (Day 17); TGI; and P value of tumor volume between the treatment and control groups.

TABLE 16

| | Tumor volume (mm³) | | | TGI % | P value |
|---|---|---|---|---|---|
| Group | Day 0 | Day 10 | Day 17 | (Day 17) | (Day 17) |
| G1 | 281 ± 16 | 943 ± 76 | 1700 ± 172 | NA | NA |
| G2 | 281 ± 20 | 146 ± 17 | 111 ± 22 | 112.0 | 1.65E−05 |
| G3 | 281 ± 21 | 139 ± 37 | 113 ± 36 | 111.8 | 1.84E−05 |
| G4 | 281 ± 25 | 129 ± 19 | 101 ± 27 | 112.7 | 1.62E−05 |
| G5 | 281 ± 25 | 148 ± 15 | 83 ± 12 | 114.0 | 1.39E−05 |
| G6 | 282 ± 33 | 542 ± 71 | 867 ± 121 | 58.8 | 0.004 |
| G7 | 281 ± 25 | 432 ± 48 | 729 ± 46 | 68.4 | 0.001 |
| G8 | 282 ± 28 | 520 ± 75 | 808 ± 114 | 62.9 | 0.003 |
| G9 | 281 ± 18 | 459 ± 51 | 767 ± 84 | 65.8 | 0.001 |

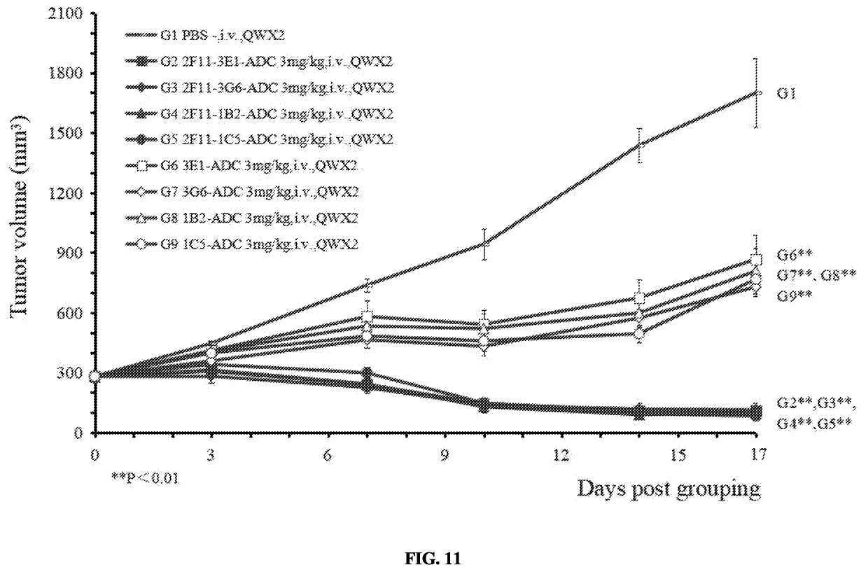

The tumor volumes of mice in different groups are shown in FIG. 11. The treatment groups (G2-G9) showed extremely significant tumor inhibition compared with PBS group (G1). In addition, the anti-HER3/MET ADCs (G2-G5) showed better tumor inhibition effects than that of the corresponding parental anti-HER3 ADCs (G6-G9).

In another similar experiment, patient-derived pancreatic tumor fragments (2 mm×2 mm×2 mm) were engrafted in the right flank of B-NDG mice. When the tumors in the mice reached a volume of about 250-300 mm³, the mice were randomly placed into different groups based on tumor volumes (5 mice per group), and then injected with PBS, ISO-ADC, Patritumab-ADC, 2F11-3E1-ADC, 2F11-3G6-ADC, 2F11-1B2-ADC or 2F11-1C5-ADC by i.v. administration. The tumor volumes were measured twice a week and body weights of the mice were recorded as well.

The weight of mice in different groups all increased. On Day 0, the average weight of each group was in the range of 22.3 g-25.9 g. At the end of the experiment (Day 42), the average weight of each group was in the range of 22.9 g-27.0 g. The average weight of each group was in the range of 100.6%-104.1%. The results showed that the tested ADCs were well tolerated and were not obviously toxic to the mice.

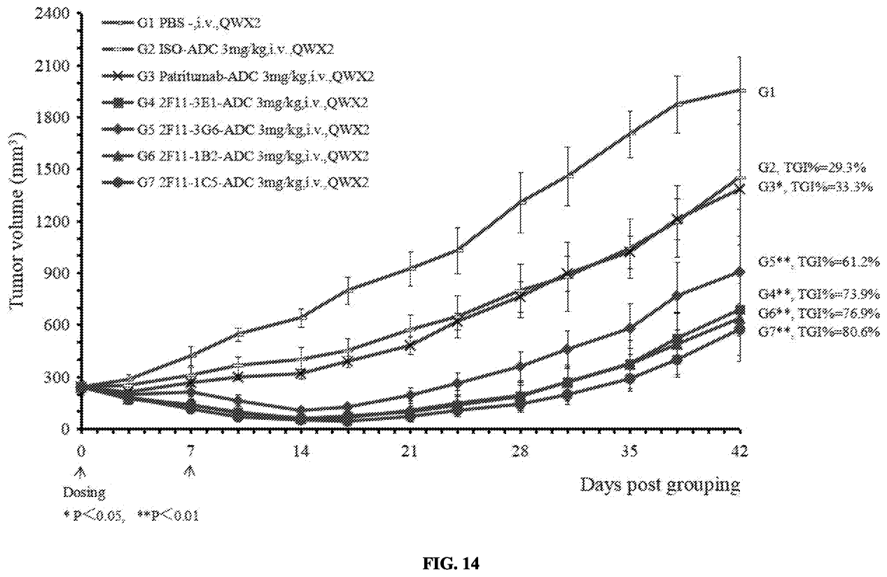

As shown in FIG. 14, 2F11-3E1-ADC (G4), 2F11-3G6-ADC (G5), 2F11-1B2-ADC (G6) and 2F11-1C5-ADC (G7) induced significant tumor growth inhibition at 3 mg/kg with a higher TGI % (e.g., on Day 42) than that of the positive control Patritumab-ADC (G3).

### Example 12. In Vitro Killing Activities of Anti-HER3 ADCs

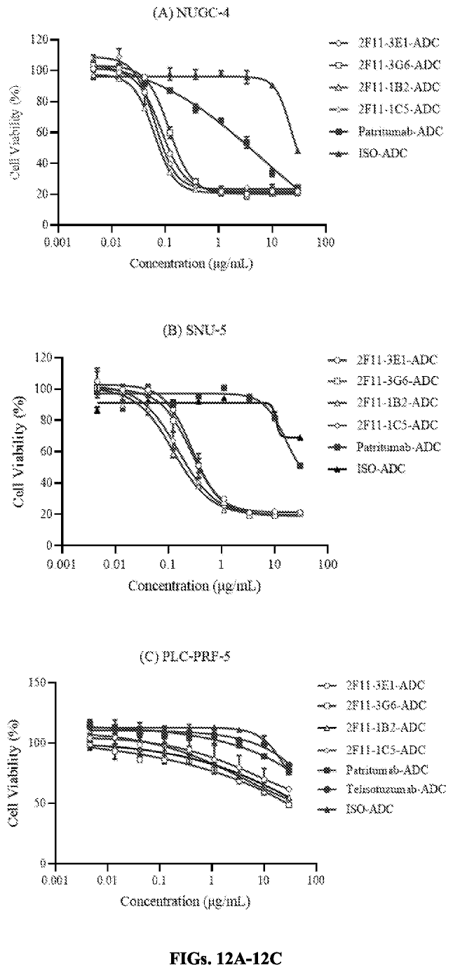

Different concentrations of anti-HER3 ADCs (30 μg/mL, 10 μg/mL, 3.333 μg/mL, 1.111 μg/mL, 0.370 μg/mL, 0.123 μg/mL, 0.041 μg/mL, 0.014 μg/mL, and 0.005 μg/mL) were used to treat NUGC-4 cells (HER3^{high}MET^{moderate}), SNU-5 cells (HER3^{low}MET^{high}) or PLC-PRF-5 cells (HER3^{low}MET^{low}) cultured in 96-well plates respectively. After 72 hours incubation, PrestoBlue™ (Thermofisher, Cat #: A13261) was added to the 96-well plates. The cell viability was detected after 2 hours incubation via a microplate reader. The results are shown in FIGS. 12A-12C.

The above results showed that, compared with the positive controls Patritumab-ADC or Telisotuzumab-ADC, all the four anti-HER3/MET ADCs exhibited better in vitro killing activities in human gastric cancer cell lines (NUGC-4 and SNU-5) and human liver carcinoma cell line (PLC-PRF-5).

### Example 13. Anti-Tumor Activity of Anti-HER3 ADC in HCC827 Model

The ADCs were tested for their effect on tumor growth in vivo in a lung cancer HCC827 xenograft model. Specifically, about 1×10⁷ HCC827 cells were injected subcutaneously in B-NDG mice. When the tumors in the mice reached a volume of about 300 mm³, the mice were randomly placed into different groups based on tumor volumes, and then injected with PBS, Patritumab-ADC, 2F11-3E1-ADC, 2F11-3G6-ADC, 2F11-1B2-ADC or 2F11-1C5-ADC by i.v. administration. The frequency of administration was once a week (2 administrations in total).

The results showed that, 2F11-3E1-ADC, 2F11-3G6-ADC, 2F11-1B2-ADC and 2F11-1C5-ADC inhibited tumor growth with a higher TGI % (e.g., on Day 27) than that of the positive control Patritumab-ADC. For example, when administered with a dose level of 1.5 mg/kg on Day 0 and 3 mg/kg on Day 7, the TGI % in 2F11-3E1-ADC, 2F11-3G6-ADC, 2F11-1B2-ADC and 2F11-1C5-ADC treatment groups were 57.8%, 70.0%, 70.1% and 53.2% respectively, while it was 15.0% in Patritumab-ADC treatment group. 2F11-3E1-ADC, 2F11-3G6-ADC, 2F11-1B2-ADC and 2F11-1C5-ADC have good tumor inhibitory effects in lung cancer model.

### Example 14. Anti-Tumor Activity of Anti-HER3 ADC in HT29 Model

The ADCs were tested for their effect on tumor growth in vivo in a colorectal cancer HT29 xenograft model. Specifically, about 5×10⁶ HT29 cells were injected subcutaneously in B-NDG mice. When the tumors in the mice reached a volume of about 200 mm³, the mice were randomly placed into different groups based on tumor volumes, and then injected with PBS, Patritumab-ADC, 2F11-3G6-ADC, 2F11-1B2-ADC, 2F11-1C5-ADC, 2F11-ADC, 3G6-ADC, 1B2-ADC, or 1C5-ADC by i.v. administration. The frequency of administration was once a week (2 administrations in total).

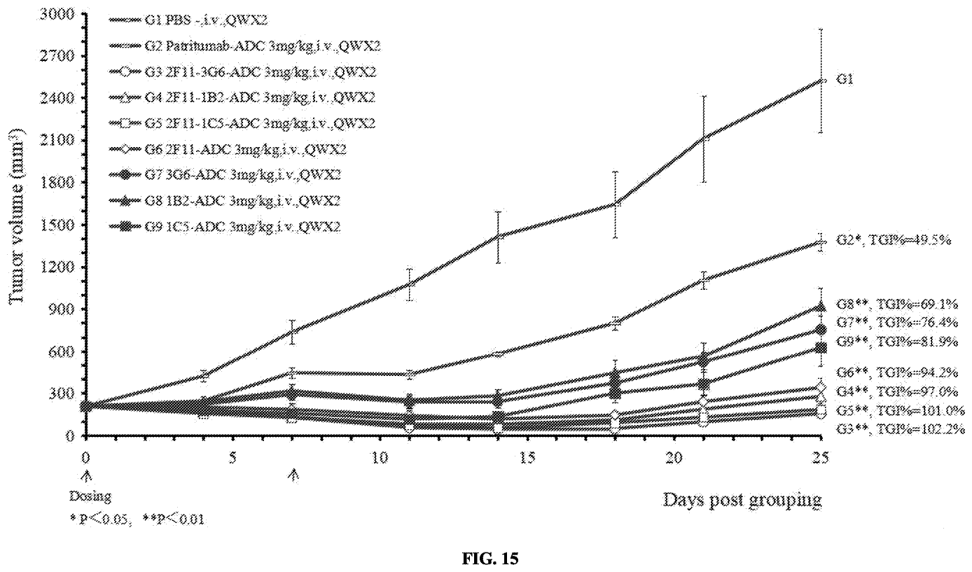

As shown in FIG. 15, all the bispecific ADCs 2F11-3G6-ADC (G3), 2F11-1B2-ADC (G4) and 2F11-1C5-ADC (G5) exhibited the best tumor inhibitory effects in the colorectal cancer model compared with the parental monoclonal antibodies or the positive control. In addition, the corresponding parental anti-HER3 monoclonal antibodies 3G6-ADC (G7), 1B2-ADC (G8) and 1C5-ADC (G9) also obtain better tumor inhibitory effects than that of the positive control Patritumab-ADC (G2).

### Example 15. Antibody Drug Conjugates

The purified antibodies were also coupled with CPT-1, CPT-2, CPT-3, or CPT-4, through a CPT-L linker. For the names of antibody-drug conjugates, CPTx (x=1, 2, 3, or 4) is added directly after the antibody name. For example, if 3E1 is coupled to CPT-1, it is named as 3E1-CPT1. As another example, if 3E1 is coupled to CPT-2, it is named as 3E1-CPT2. MS (Mass Spectrometry) was used to detect the coupling of antibodies with drug molecules. Human IgG1 was coupled to CPT-2 for the isotype control, and named as ISO-CPT2. The MS detection results showed that the drug-to-antibody ratio (DAR) of the ADCs was about 8.

Human IgG1 and Patritumab analog were coupled with Dxd through GGFG linker, for comparative purposes, and the resulting ADC were named ISO-Dxd and Patritumab-Dxd respectively.

### Example 16. Anti-Tumor Activity of Anti-HER3 ADC in Pancreatic PDX Model

The ADCs were tested for their effect on tumor growth in vivo in a pancreatic cancer PDX model. Specifically, patient-derived pancreatic tumor fragments (2 mm×2 mm×2 mm) were engrafted in the right flank of B-NDG mice. When the tumors in the mice reached a volume of about 200-300 mm³, the mice were randomly placed into different groups based on tumor volumes (5 mice per group), and then injected with PBS, Patritumab-Dxd, Telisotuzumab-ADC, 2F11-3E1-CPT2, 1B2-2F11-CPT2, or 2F11-1C5-CPT2 by i.v. administration. The frequency of administration was once a week (1 administrations in total).

The results showed that, 2F11-3E1-CPT2, 1B2-2F11-CPT2 and 2F11-1C5-CPT2 inhibited tumor growth with a higher TGI % (e.g., on Day 32) than that of the positive control Patritumab-Dxd. For example, when administered with a dose level of 6 mg/kg on Day 0, the TGI % in 2F11-3E1-CPT2, 1B2-2F11-CPT2 and 2F11-1C5-CPT2 treatment groups were 76.5%, 75.7% and 78.1% respectively, while it was 34.4% in Patritumab-Dxd treatment group with a dose level of 10 mg/kg. 2F11-3E1-CPT2, 1B2-2F11-CPT2 and 2F11-1C5-CPT2 have good tumor inhibitory effects in pancreatic cancer model.

In another experiment, similar procedure was performed, except that the mice were injected with PBS (G1), Patritumab-Dxd (G2), Telisotuzumab-ADC (G3), 3E1-8D9-CPT2 (G4), 1B2-8D9-CPT2 (G5), or 1C5-8D9-CPT2 (G6). The table below summarizes the results for this experiment, including the tumor volumes on the day of grouping (Day 0), 14 days after grouping (Day 14) and 28 days after grouping (Day 28), TGI and P value of tumor volume between the treatment and control groups. The results showed that 3E1-8D9-CPT2 (G4), 1B2-8D9-CPT2 (G5) and 1C5-8D9-CPT2 (G6) inhibited tumor growth with a higher TGI % than the positive controls Patritumab-Dxd (G2) and Telisotuzumab-ADC (G3).

TABLE 17

| | Tumor volume (mm³) | | | TGI % | P value |
|---|---|---|---|---|---|
| Group | Day 0 | Day 14 | Day 28 | (Day 28) | (Day 28) |
| G1 | 218 ± 26 | 763 ± 60 | 1617 ± 103 | NA | NA |
| G2 | 218 ± 30 | 462 ± 78 | 1164 ± 188 | 32.4 | 0.067 |
| G3 | 218 ± 32 | 82 ± 23 | 435 ± 104 | 84.5 | 4.07E−05 |
| G4 | 218 ± 22 | 68 ± 10 | 367 ± 41 | 89.4 | 3.32E−06 |
| G5 | 218 ± 32 | 104 ± 47 | 423 ± 116 | 85.3 | 5.69E−05 |
| G6 | 218 ± 36 | 84 ± 24 | 306 ± 66 | 93.7 | 4.98E−06 |

### Example 17. Anti-Tumor Activity of Anti-HER3 ADC in Gastric PDX Model

The ADCs were tested for their effect on tumor growth in vivo in a gastric cancer PDX model. Specifically, patient-derived gastric tumor fragments (2 mm×2 mm×2 mm) were engrafted in the right flank of B-NDG mice. When the tumors in the mice reached a volume of about 200-300 mm³, the mice were randomly placed into different groups based on tumor volumes (5 mice per group), and then injected with PBS, Patritumab-Dxd, Telisotuzumab-ADC, 1B2-2F11-CPT2, or 2F11-1C5-CPT2 by i.v. administration. The frequency of administration was once a week (2 administrations in total).

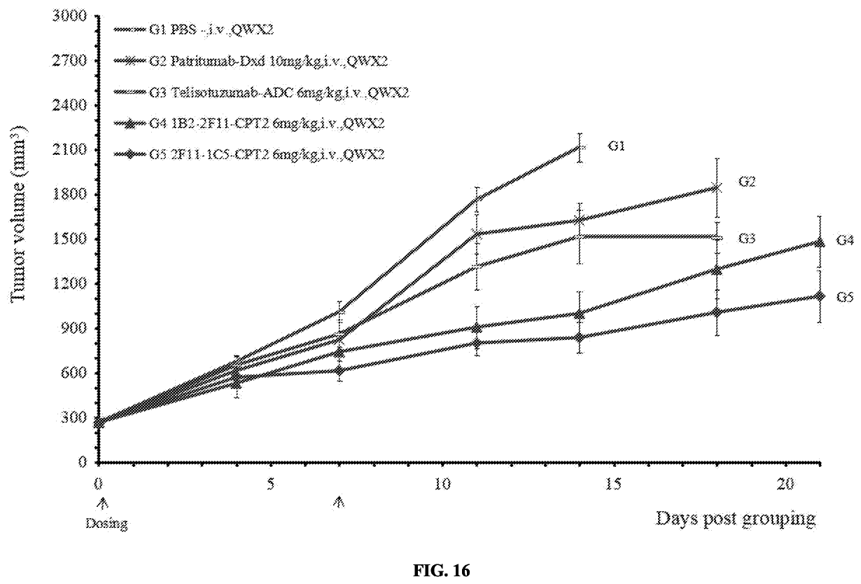

As shown in FIG. 16, the tumor volumes in all treatment groups (G2-G5) were smaller than those in the control group (G1). The results also showed that 1B2-2F11-CPT2 (G4) and 2F11-1C5-CPT2 (G5) had better tumor inhibitory effects a dose level of 6 mg/kg than that of the positive controls Patritumab-Dxd (G2) and Telisotuzumab-ADC (G3).

On Day 18, all mice in G1 group died, and one mice died in G2 and G3 groups. At the end of the experiment, all mice died in G2 and G3 groups, while all mice in G4 and G5 groups survived, indicating that 1B2-2F11-CPT2 (G4) and 2F11-1C5-CPT2 (G5) have excellent tumor inhibitory effect and good safety.

In another similar experiment, gastric cancer PDX model mice were injected with PBS (G1), Patritumab-Dxd (G2), Telisotuzumab-ADC (G3), 3E1-8D9-CPT2 (G4), 1B2-8D9-CPT2 (G5), 1C5-8D9-CPT2 (G6), 3E1-8H10-CPT2 (G7), 1B2-8H10-CPT2 (G8), or 1C5-8H10-CPT2 (G9). The table below summarizes the results for this experiment, including the tumor volumes on the day of grouping (Day 0), 7 days after grouping (Day 7) and 14 days after grouping (Day 14), TGI and P value of tumor volume between the treatment and control groups. The results showed that the anti-HER3/MET bispecific ADCs (G4 to G9) inhibited tumor growth with a higher TGI % than the positive controls Patritumab-Dxd (G2) and Telisotuzumab-ADC (G3).

TABLE 18

| | Tumor volume (mm³) | | | TGI % | P value |
|---|---|---|---|---|---|
| Group | Day 0 | Day 7 | Day 14 | (Day 14) | (Day 14) |
| G1 | 266 ± 11 | 1008 ± 71 | 2113 ± 96 | NA | NA |
| G2 | 266 ± 28 | 820 ± 44 | 1622 ± 118 | 26.6 | 0.014 |
| G3 | 265 ± 29 | 859 ± 100 | 1513 ± 179 | 32.4 | 0.018 |
| G4 | 265 ± 23 | 689 ± 80 | 937 ± 203 | 63.6 | 0.001 |
| G5 | 265 ± 27 | 585 ± 106 | 729 ± 163 | 74.9 | 8.14E−05 |
| G6 | 265 ± 23 | 687 ± 65 | 1029 ± 170 | 58.6 | 0.001 |
| G7 | 266 ± 26 | 706 ± 110 | 1098 ± 169 | 55.0 | 0.001 |
| G8 | 265 ± 28 | 746 ± 135 | 902 ± 170 | 65.5 | 2.56E−04 |
| G9 | 266 ± 28 | 759 ± 116 | 1104 ± 130 | 54.6 | 2.49E−04 |

### Example 18. Anti-Tumor Activity of Anti-HER3 ADC in Colorectal Cancer PDX Model

The ADCs were tested for their effect on tumor growth in vivo in a colorectal cancer PDX model. Specifically, patient-derived colorectal tumor fragments (2 mm×2 mm×2 mm) were engrafted in the right flank of B-NDG mice. When the tumors in the mice reached a volume of about 250 mm³, the mice were randomly placed into different groups based on tumor volumes (5 mice per group), and then injected with PBS (G1), ISO-ADC (G2), ISO-Dxd (G3), ISO-CPT2 (G4), Patritumab-Dxd (G5), Telisotuzumab-ADC (G6), 2F11-3E1-CPT2 (G7) or 3E1-8D9-CPT2 (G8) by i.v. administration with equal molar dosage. The frequency of administration was once a week (2 administrations in total).

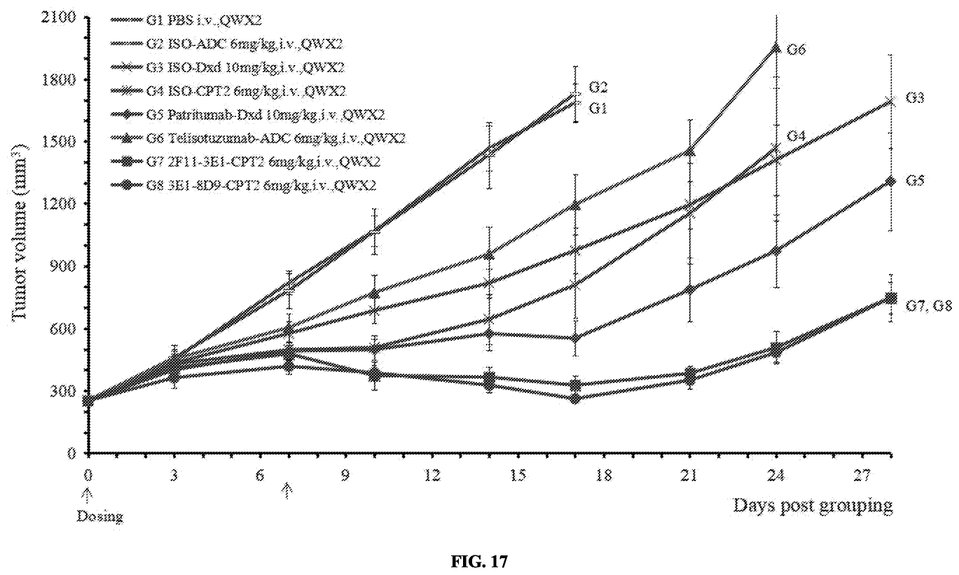

As shown in FIG. 17, 2F11-3E1-CPT2 (G7) and 3E1-8D9-CPT2 (G8) exhibited better tumor inhibitory effects than that of the positive controls Patritumab-Dxd (G5) and Telisotuzumab-ADC (G6).

In another similar experiment, patient-derived colorectal tumor fragments were engrafted in the right flank of B-NDG mice. When the tumors in the mice reached a volume of about 250 mm³, the mice were randomly placed into different groups based on tumor volumes (5 mice per group), and then injected with PBS (G1), ISO-Dxd (G2), Patritumab-Dxd (G3), 2F11-3E1-CPT2 (G4), 3E1-CPT2 (G5) or 3E1-8D9-CPT2 (G6) by i.v. administration with equal molar dosage.

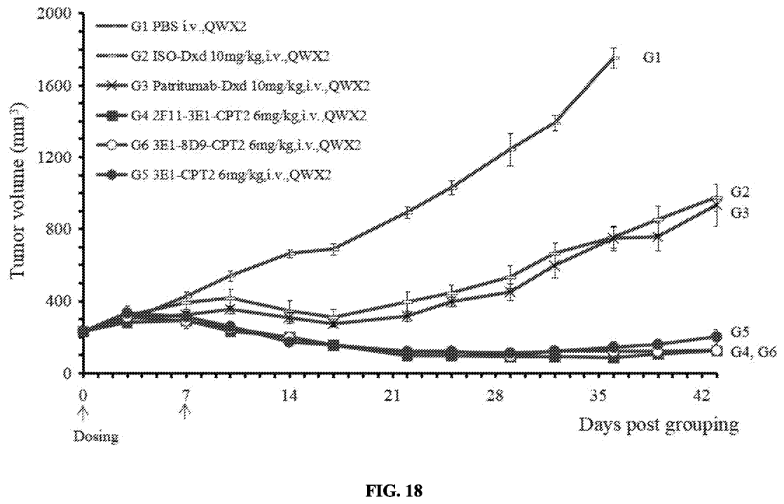

As shown in FIG. 18, 2F11-3E1-CPT2 (G4), 3E1-8D9-CPT2 (G6) and 3E1-CPT2 (G5) showed better tumor growth inhibition effects than that of the positive controls Patritumab-Dxd (G3). In addition, 2F11-3E1-CPT2 (G4) and 3E1-8D9-CPT2 (G6) exhibited better anti-tumor activities than that of the corresponding monoclonal ADC 3E1-CPT2 (G5).

### Example 19. Anti-Tumor Activity of Anti-HER3 ADC in Lung Cancer PDX Model

The ADCs were tested for their effect on tumor growth in vivo in a lung cancer PDX model. Specifically, patient-derived lung tumor fragments (2 mm×2 mm×2 mm) were engrafted in the right flank of B-NDG mice. When the tumors in the mice reached a volume of about 250 mm³, the mice were randomly placed into different groups based on tumor volumes (5 mice per group), and then injected with PBS, ISO-ADC, ISO-Dxd, ISO-CPT2, Patritumab-Dxd, Telisotuzumab-ADC, or 2F11-3E1-CPT2 (G7) with equal molar dosage by i.v. administration. The frequency of administration was once a week (2 administrations in total).

Similar to the previous results, the experimental results showed that 2F11-3E1-CPT2 exhibited better anti-tumor activities than that of the positive controls Patritumab-Dxd and Telisotuzumab-ADC.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to HER3 (human epidermal growth factor receptor 3) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 19-21, respectively;
(7) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 19-21, respectively;
(8) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 19-21, respectively;
(9) the selected VH CDRs 1, 2, 3 amino acid sequences are set forth in SEQ ID NOs: 31-33, respectively, and the selected VL CDRs 1, 2, 3 amino acid sequences are set forth in SEQ ID NOs: 19-21, respectively; and
(10) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 19-21, respectively.

2. The antibody or antigen-binding fragment thereof of claim 1, wherein the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 1-3, respectively, according to Kabat definition.

3. The antibody or antigen-binding fragment thereof of claim 1, wherein the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 1-3, respectively, according to Kabat definition.

4. The antibody or antigen-binding fragment thereof of claim 1, wherein the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 1-3, respectively, according to Kabat definition.

5. The antibody or antigen-binding fragment thereof of claim 1, wherein the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 1-3, respectively, according to Kabat definition.

6. The antibody or antigen-binding fragment thereof of claim 1, wherein the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 1-3, respectively, according to Kabat definition.

7. The antibody or antigen-binding fragment thereof of claim 1, wherein the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 19-21, respectively, according to Chothia definition.

8. The antibody or antigen-binding fragment thereof of claim 1, wherein the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 19-21, respectively, according to Chothia definition.

9. The antibody or antigen-binding fragment thereof of claim 1, wherein the VH comprises CDRs 1, 2, 3 with the amino acid sequences set forth in SEQ ID NOs: 28-30, respectively, and the VL comprises CDRs 1, 2, 3 with the amino acid sequences set forth in SEQ ID NOs: 19-21, respectively, according to Chothia definition.

10. The antibody or antigen-binding fragment thereof of claim 1, wherein the VH comprises CDRs 1, 2, 3 with the amino acid sequences set forth in SEQ ID NOs: 31-33, respectively, and the VL comprises CDRs 1, 2, 3 with the amino acid sequences set forth in SEQ ID NOs: 19-21, respectively, according to Chothia definition.

11. The antibody or antigen-binding fragment thereof of claim 1, wherein the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 19-21, respectively, according to Chothia definition.

12. The antibody or antigen-binding fragment thereof of any one of claims 1-11, wherein the antibody or antigen-binding fragment thereof specifically binds to human, mouse, monkey, or dog HER3.

13. The antibody or antigen-binding fragment thereof of any one of claims 1-12, wherein the antibody or antigen-binding fragment thereof is a human antibody or antigen-binding fragment thereof, a single-chain variable fragment (scFv), a one-armed antibody, and/or a multi-specific antibody (e.g., a bispecific antibody).

14. The antibody or antigen-binding fragment thereof of any one of claims 1-13, wherein the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

15. An antibody or antigen-binding fragment thereof that binds to HER3 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

16. The antibody or antigen-binding fragment thereof of claim 15, wherein the VH comprises the sequence of SEQ ID NO: 38 and the VL comprises the sequence of SEQ ID NO: 37.

17. The antibody or antigen-binding fragment thereof of claim 15, wherein the VH comprises the sequence of SEQ ID NO: 39 and the VL comprises the sequence of SEQ ID NO: 37.

18. The antibody or antigen-binding fragment thereof of claim 15, wherein the VH comprises the sequence of SEQ ID NO: 40 and the VL comprises the sequence of SEQ ID NO: 37.

19. The antibody or antigen-binding fragment thereof of claim 15, wherein the VH comprises the sequence of SEQ ID NO: 41 and the VL comprises the sequence of SEQ ID NO: 37.

20. The antibody or antigen-binding fragment thereof of claim 15, wherein the VH comprises the sequence of SEQ ID NO: 42 and the VL comprises the sequence of SEQ ID NO: 37.

21. An antibody or antigen-binding fragment thereof that binds to HER3 comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

22. The antibody or antigen-binding fragment thereof of any one of claims 1-21, wherein the antibody or antigen-binding fragment thereof specifically binds to human, mouse, monkey, or dog HER3.

23. The antibody or antigen-binding fragment thereof of any one of claims 1-22, wherein the antibody or antigen-binding fragment thereof is a human antibody or antigen-binding fragment thereof, a single-chain variable fragment (scFv), a one-armed antibody, and/or a multi-specific antibody (e.g., a bispecific antibody).

24. The antibody or antigen-binding fragment thereof of any one of claims 1-23, wherein the antibody or antigen-binding fragment is a human IgG1 antibody or antigen-binding fragment thereof, a human IgG2 antibody or antigen-binding fragment thereof, or a human IgG4 antibody or antigen-binding fragment thereof.

25. An antibody or antigen-binding fragment thereof, comprising: a first antigen-binding domain that specifically binds to a first epitope of HER3; and a second antigen-binding domain that specifically binds to a second epitope of HER3.

26. The antibody or antigen-binding fragment thereof of claim 25, wherein the first antigen-binding and the second antigen-binding domain comprises a second heavy chain variable region (VH2) and a second light chain variable region (VL2).

27. The antibody or antigen-binding fragment thereof of claim 26, wherein
the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively;
(7) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 19-21, respectively;
(8) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 19-21, respectively;
(9) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively; and
(10) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively.

28. The antibody or antigen-binding fragment thereof of claim 27, wherein the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively.

29. The antibody or antigen-binding fragment thereof of claim 27, wherein the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively.

30. The antibody or antigen-binding fragment thereof of claim 2, wherein the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively.

31. The antibody or antigen-binding fragment thereof of claim 27, wherein the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively.

32. The antibody or antigen-binding fragment thereof of claim 27, wherein the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively.

33. The antibody or antigen-binding fragment thereof of any one of claims 26-32, wherein
the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 4-6, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 10-12, respectively, and the selected VL2 CDRs 1, 2, 3 amino acid sequences are set forth in SEQ ID NOs: 1-3, respectively;
(4) the selected VH2 CDRs 1, 2, 3 amino acid sequences are set forth in SEQ ID NOs: 13-15, respectively, and the selected VL2 CDRs 1, 2, 3 amino acid sequences are set forth in SEQ ID NOs: 1-3, respectively;
(5) the selected VH2 CDRs 1, 2, 3 amino acid sequences are set forth in SEQ ID NOs: 16-18, respectively, and the selected VL2 CDRs 1, 2, 3 amino acid sequences are set forth in SEQ ID NOs: 1-3, respectively;
(6) the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 19-21, respectively;
(7) the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively;
(8) the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 19-21, respectively;
(9) the selected VH2 CDRs 1, 2, 3 amino acid sequences are set forth in SEQ ID NOs: 31-33, respectively, and the selected VL2 CDRs 1, 2, 3 amino acid sequences are set forth in SEQ ID NOs: 19-21, respectively; and
(10) the selected VH2 CDRs 1, 2, 3 amino acid sequences are set forth in SEQ ID NOs: 34-36, respectively, and the selected VL2 CDRs 1, 2, 3 amino acid sequences are set forth in SEQ ID NOs: 19-21, respectively.

34. The antibody or antigen-binding fragment thereof of any one of claims 26-33, wherein the selected VH2 CDRs 1, 2, 3 amino acid sequences are set forth in SEQ ID NOs: 4-6, respectively, and the selected VL2 CDRs 1, 2, 3 amino acid sequences are set forth in SEQ ID NOs: 1-3, respectively.

35. The antibody or antigen-binding fragment thereof of any one of claims 26-33, wherein the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively.

36. The antibody or antigen-binding fragment thereof of any one of claims 26-33, wherein the selected VH2 CDRs 1, 2, 3 amino acid sequences are set forth in SEQ ID NOs: 10-12, respectively, and the selected VL2 CDRs 1, 2, 3 amino acid sequences are set forth in SEQ ID NOs: 1-3, respectively.

37. The antibody or antigen-binding fragment thereof of any one of claims 26-33, wherein the selected VH2 CDRs 1, 2, 3 amino acid sequences are set forth in SEQ ID NOs: 13-15, respectively, and the selected VL2 CDRs 1, 2, 3 amino acid sequences are set forth in SEQ ID NOs: 1-3, respectively.

38. The antibody or antigen-binding fragment thereof of any one of claims 26-33, wherein the selected VH2 CDRs 1, 2, 3 amino acid sequences are set forth in SEQ ID NOs: 16-18, respectively, and the selected VL2 CDRs 1, 2, 3 amino acid sequences are set forth in SEQ ID NOs: 1-3, respectively.

39. The antibody or antigen-binding fragment thereof of claim 26 or claim 27, wherein
(1) The selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively;
(2) The selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively;
(3) The selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively;
(4) The selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively;
(5) The selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 7-9, respectively, and the selected VL2 CDRs 1, 2, 3 amino acid sequences are set forth in SEQ ID NOs: 1-3, respectively; or
(6) The selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 19-21, respectively.

40. The antibody or antigen-binding fragment thereof of any one of claims 26-39, wherein the VH1 comprises an amino acid sequence that is at least 80%, 85%, 90%, 95%, 99%, or 100% identical to a selected VH sequence, and the VL1 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

41. The antibody or antigen-binding fragment thereof of any one of claims 26-40, wherein the VH2 comprises an amino acid sequence that is at least 80%, 85%, 90%, 95%, 99%, or 100% identical to a selected VH sequence, and the VL2 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

42. The antibody or antigen-binding fragment thereof of claim 26 or claim 27, wherein the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

43. The antibody or antigen-binding fragment thereof of claim 26 or claim 27, wherein the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

44. An antibody or antigen-binding fragment thereof, comprising: a first antigen-binding domain that specifically binds to HER3; and a second antigen-binding domain that specially binds to MET.

45. The antibody or antigen-binding fragment thereof of claim 44, wherein the first antigen-binding and the second antigen-binding domain comprises a second heavy chain variable region (VH2) and a second light chain variable region (VL2).

46. The antibody or antigen-binding fragment thereof of claim 45, wherein
the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 19-21, respectively;
(7) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 19-21, respectively;
(8) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 19-21, respectively;
(9) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 19-21, respectively; and
(10) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 19-21, respectively.

47. The antibody or antigen-binding fragment thereof of claim 45 or claim 46, wherein the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 19-21, respectively;
(4) the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 19-21, respectively;
(5) the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(6) the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively;
(7) the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 19-21, respectively; and
(8) the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 19-21, respectively.

48. The antibody or antigen-binding fragment thereof of any one of claims 45-47, wherein
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively;
(15) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively; or
(16) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 1-3, respectively.

49. The antibody or antigen-binding fragment thereof of any one of claims 45-48, wherein the VH1 comprises an amino acid sequence that is at least 90% identical to a selected VH sequence, and the VL1 comprises an amino acid sequence that is at least 90% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37; and
(5) the selected VH sequence is SEQ ID NO: 42, and the selected VL sequence is SEQ ID NO: 37.

50. The antibody or antigen-binding fragment thereof of any one of claims 45-49, wherein the VH2 comprises an amino acid sequence that is at least 90% identical to a selected VH sequence, and the VL2 comprises an amino acid sequence that is at least 90% identical to a selected VL sequence, wherein the selected VH sequence and the selected VL sequence are one of the following:
(1) the selected VH sequence is SEQ ID NO: 63, and the selected VL sequence is SEQ ID NO: 37;
(2) the selected VH sequence is SEQ ID NO: 64, and the selected VL sequence is SEQ ID NO: 37;
(3) the selected VH sequence is SEQ ID NO: 81, and the selected VL sequence is SEQ ID NO: 37; and
(4) the selected VH sequence is SEQ ID NO: 82, and the selected VL sequence is SEQ ID NO: 37.

51. An antibody or antigen-binding fragment thereof that cross-competes with the antibody or antigen-binding fragment thereof of any one of claims 1-50.

52. The antibody or antigen-binding fragment thereof of any one of claims 1-51, wherein the antibody or antigen-binding fragment thereof comprises a fragment crystallizable region (Fc region).

53. The antibody or antigen-binding fragment thereof of claim 52, wherein the Fc region has increased complement-dependent cytotoxicity (CDC) or antibody-dependent cellular cytotoxicity (ADCC).

54. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 4-6, respectively, and wherein the VH, when paired with a light chain variable region (VL) comprising the amino acid sequence set forth in SEQ ID NO: 37 binds to HER3;
(2) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 38 binds to HER3;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7-9, respectively, and wherein the VH, when paired with a light chain variable region (VL) comprising the amino acid sequence set forth in SEQ ID NO: 37 binds to HER3;
(4) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 39 binds to HER3;
(5) an immunoglobulin heavy chain or a fragment thereof comprising a VH comprising complementarity determining regions (CDRs) 1, 2, and 3 comprising the amino acid sequences set forth in SEQ ID NOs: 10-12, respectively, and wherein the VH, when paired with a light chain variable region (VL) comprising the amino acid sequence set forth in SEQ ID NO: 37 binds to HER3;
(6) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 40 binds to HER3;
(7) an immunoglobulin heavy chain or a fragment thereof comprising a VH comprising complementarity determining regions (CDRs) 1, 2, and 3 comprising the amino acid sequences set forth in SEQ ID NOs: 13-15, respectively, and wherein the VH, when paired with a light chain variable region (VL) comprising the amino acid sequence set forth in SEQ ID NO: 37 binds to HER3;
(8) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 41 binds to HER3;
(9) an immunoglobulin heavy chain or a fragment thereof comprising a VH comprising complementarity determining regions (CDRs) 1, 2, and 3 comprising the amino acid sequences set forth in SEQ ID NOs: 16-18, respectively, and wherein the VH, when paired with a light chain variable region (VL) comprising the amino acid sequence set forth in SEQ ID NO: 37 binds to HER3;
(10) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 42 binds to HER3;
(11) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 22-24, respectively, and wherein the VH, when paired with a light chain variable region (VL) comprising the amino acid sequence set forth in SEQ ID NO: 37 binds to HER3;
(12) an immunoglobulin light chain or a fragment thereof comprising a VL comprising complementarity determining regions (CDRs) 1, 2, and 3 comprising the amino acid sequences set forth in SEQ ID NOs: 19-21, respectively, and wherein the VL, when paired with a VH comprising the amino acid sequence set forth in SEQ ID NO: 38 binds to HER3;
(13) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25-27, respectively, and wherein the VH, when paired with a light chain variable region (VL) comprising the amino acid sequence set forth in SEQ ID NO: 37 binds to HER3;
(14) an immunoglobulin light chain or a fragment thereof comprising a VL comprising complementarity determining regions (CDRs) 1, 2, and 3 comprising the amino acid sequences set forth in SEQ ID NOs: 19-21, respectively, and wherein the VL, when paired with a VH comprising the amino acid sequence set forth in SEQ ID NO: 39 binds to HER3;
(15) an immunoglobulin heavy chain or a fragment thereof comprising a VH comprising complementarity determining regions (CDRs) 1, 2, and 3 comprising the amino acid sequences set forth in SEQ ID NOs: 28-30, respectively, and wherein the VH, when paired with a light chain variable region (VL) comprising the amino acid sequence set forth in SEQ ID NO: 37 binds to HER3;
(16) an immunoglobulin light chain or a fragment thereof comprising a VL comprising CDRs 1, 2, and 3 comprising the amino acid sequences set forth in SEQ ID NOs: 19-21, respectively, and wherein the VL, when paired with a VH comprising the amino acid sequence set forth in SEQ ID NO: 40 binds to HER3;
(17) an immunoglobulin heavy chain or a fragment thereof comprising a VH comprising complementarity determining regions (CDRs) 1, 2, and 3 comprising the amino acid sequences set forth in SEQ ID NOs: 31-33, respectively, and wherein the VH, when paired with a light chain variable region (VL) comprising the amino acid sequence set forth in SEQ ID NO: 37 binds to HER3;
(18) an immunoglobulin light chain or a fragment thereof comprising a VL comprising CDRs 1, 2, and 3 comprising the amino acid sequences set forth in SEQ ID NOs: 19-21, respectively, and wherein the VL, when paired with a VH comprising the amino acid sequence set forth in SEQ ID NO: 41 binds to HER3;
(19) an immunoglobulin heavy chain or a fragment thereof comprising a VH comprising complementarity determining regions (CDRs) 1, 2, and 3 comprising the amino acid sequences set forth in SEQ ID NOs: 34-36, respectively, and wherein the VH, when paired with a light chain variable region (VL) comprising the amino acid sequence set forth in SEQ ID NO: 37 binds to HER3; or
(20) an immunoglobulin light chain or a fragment thereof comprising a VL comprising CDRs 1, 2, and 3 comprising the amino acid sequences set forth in SEQ ID NOs: 19-21, respectively, and wherein the VL, when paired with a VH comprising the amino acid sequence set forth in SEQ ID NO: 42 binds to HER3.

55. The nucleic acid of claim 54, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1-3, respectively.

56. The nucleic acid of claim 54, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 19-21, respectively.

57. The nucleic acid of claim 54, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 4-6, respectively.

58. The nucleic acid of claim 54, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7-9, respectively.

59. The nucleic acid of claim 54, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 10-12, respectively.

60. The nucleic acid of claim 54, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13-15, respectively.

61. The nucleic acid of claim 54, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 16-18, respectively.

62. The nucleic acid of claim 54, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 22-24, respectively.

63. The nucleic acid of claim 54, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 25-27, respectively.

64. The nucleic acid of claim 54, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 28-30, respectively.

65. The nucleic acid of claim 54, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 31-33, respectively.

66. The nucleic acid of claim 54, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 34-36, respectively.

67. The nucleic acid of any one of claims 54-66, wherein the VH when paired with a VL specifically binds to human, mouse, monkey, or dog HER3, or the VL when paired with a VH specifically binds to human, mouse, monkey, or dog HER3.

68. The nucleic acid of any one of claims 54-67, wherein the immunoglobulin heavy chain or the fragment thereof is a human immunoglobulin heavy chain or a fragment thereof (e.g., a human IgG1 heavy chain or a fragment thereof, a human IgG2 antibody or antigen-binding fragment thereof, or a human IgG4 heavy chain or a fragment thereof), and the immunoglobulin light chain or the fragment thereof is a human immunoglobulin light chain or a fragment thereof.

69. The nucleic acid of any one of claims 54-68, wherein the nucleic acid encodes a single-chain variable fragment (scFv), a multi-specific antibody (e.g., a bispecific antibody), or a chimeric antigen receptor (CAR).

70. The nucleic acid of any one of claims 54-69, wherein the nucleic acid is cDNA.

71. A nucleic acid encodes the antibody or antigen-binding fragment thereof of any one of claims 1-53.

72. A vector comprising one or more of the nucleic acids of any one of claims 54-71.

73. A vector comprising two of the nucleic acids of any one of claims 54-71, wherein the vector encodes the VL region and the VH region that together bind to HER3.

74. A pair of vectors, wherein each vector comprises one of the nucleic acids of any one of claims 54-71, wherein together the pair of vectors encodes the VL region and the VH region that together bind to HER3.

75. A cell comprising the vector of claim 72 or 73, or the pair of vectors of claim 74.

76. The cell of claim 75, wherein the cell is a CHO cell.

77. A cell comprising one or more of the nucleic acids of any one of claims 54-71.

78. A method of producing an antibody or an antigen-binding fragment thereof, the method comprising
(a) culturing the cell of any one of claims 75-77 under conditions sufficient for the cell to produce the antibody or the antigen-binding fragment; and
(b) collecting the antibody or the antigen-binding fragment produced by the cell.

79. A chimeric antigen receptor (CAR) comprising the antibody or antigen-binding fragment thereof of any one of claims 1-53.

80. An antibody-drug conjugate comprising the antibody or antigen-binding fragment thereof of any one of claims 1-53 covalently bound to a therapeutic agent.

81. The antibody drug conjugate of claim 80, wherein the therapeutic agent is a cytotoxic or cytostatic agent.

82. The antibody-drug conjugate of claim 80 or 81, wherein the therapeutic agent is MMAE or MMAF.

83. The antibody-drug conjugate of claim 80, wherein the therapeutic agent is selected from

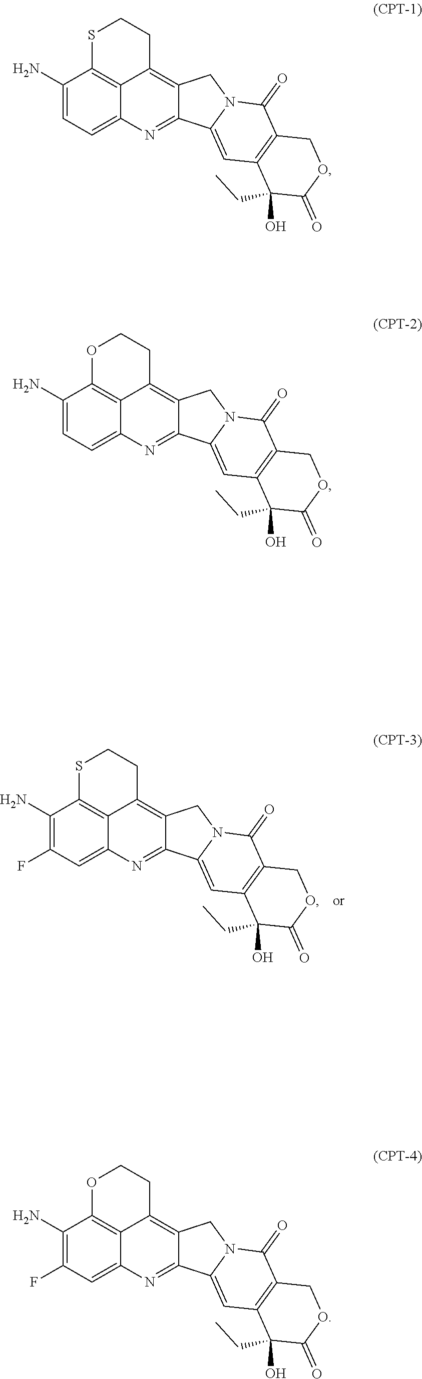 [SMILES: CC[C@@]1(O)C(=O)OCc2c1cc1n(c2=O)Cc2c-1nc1ccc(N)c3c1c2CCS3 ; CC[C@@]1(O)C(=O)OCc2c1cc1n(c2=O)Cc2c-1nc1ccc(N)c3c1c2CCO3 ; CC[C@@]1(O)C(=O)OCc2c1cc1n(c2=O)Cc2c-1nc1cc(F)c(N)c3c1c2CCS3 ; CC[C@@]1(O)C(=O)OCc2c1cc1n(c2=O)Cc2c-1nc1cc(F)c(N)c3c1c2CCO3]

84. The antibody-drug conjugate of claim 80 or 83, wherein the therapeutic agent is linked to the antibody or antigen-binding fragment thereof via a linker.

85. The antibody-drug conjugate of any one of claims 80, 83, and 84, wherein the linker has a structure of:

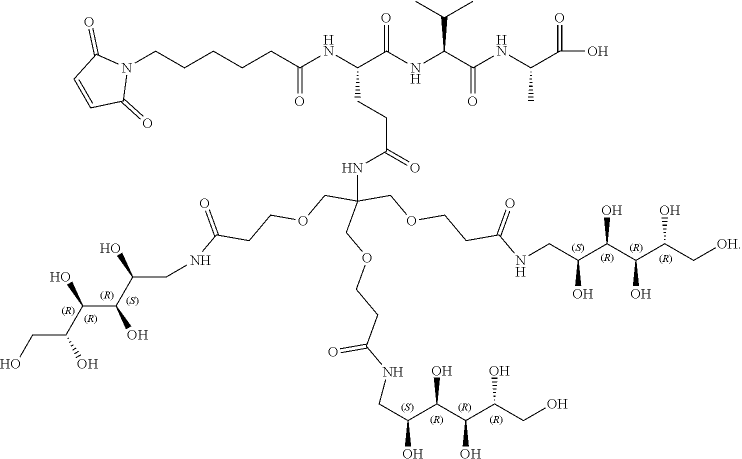

86. The antibody-drug conjugate of any one of claims 80 and 83-85, wherein the antibody-drug conjugate has a structure of: 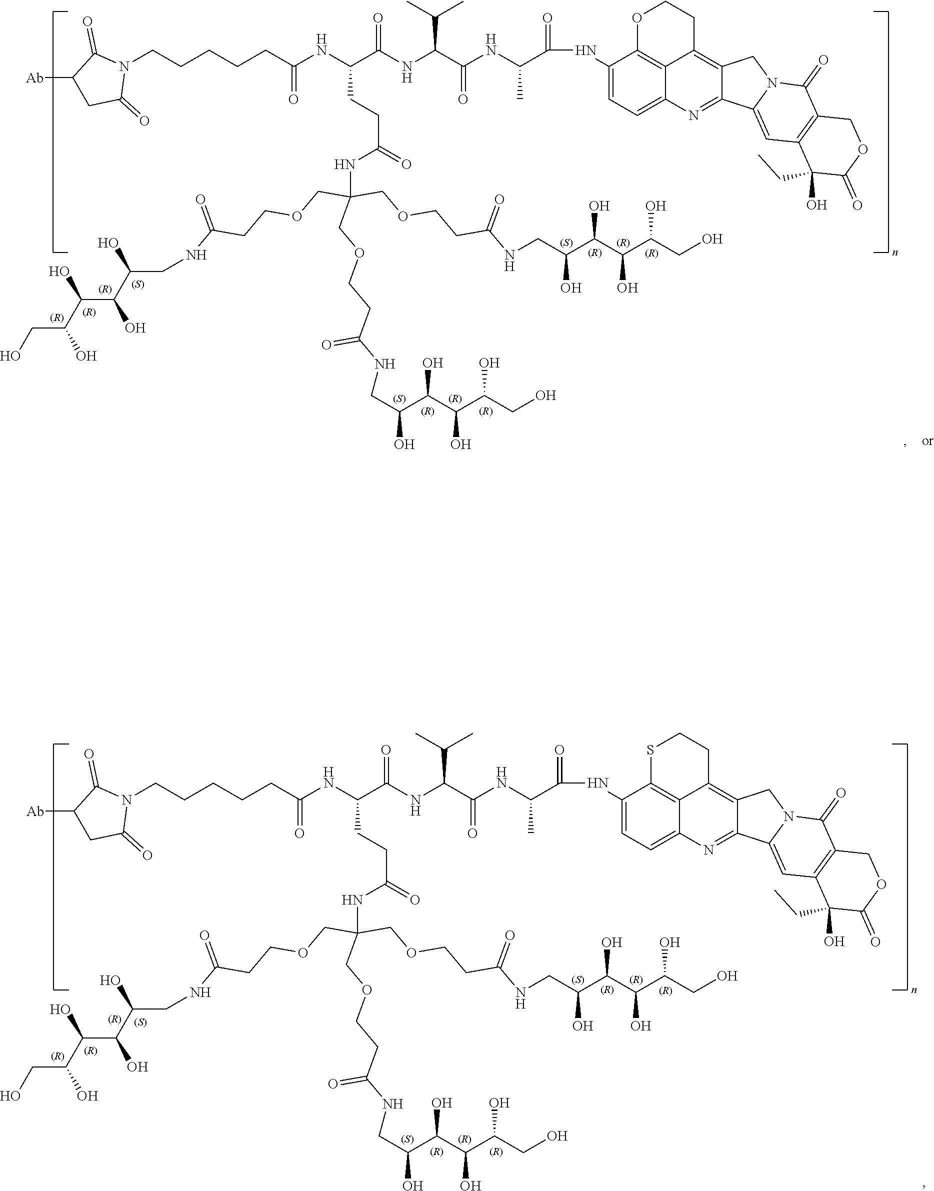
wherein n=1, 2, 3, 4, 5, 6, 7, or 8; wherein “Ab” represents the antibody or antigen-binding fragment thereof.

87. A method of treating a subject having cancer, the method comprising administering a therapeutically effective amount of a composition comprising the antibody or antigen-binding fragment thereof of any one of claims 1-53, the CAR of claim 79, or the antibody-drug conjugate of any one of claims 80-86, to the subject.

88. The method of claim 87, wherein the subject has a solid tumor.

89. The method of claim 87, wherein the cancer is colorectal cancer, gastric cancer, breast cancer, lung cancer, melanoma, ovarian cancer, head and neck cancer, pancreatic cancer, liver carcinoma or cervical cancer.

90. The method of claim 87, wherein the subject is further treated with an effective amount of an anti-4-1BB antibody, an anti-OX40 antibody, an anti-PD-1 antibody, an anti-CTLA4 antibody, an anti-CD40 antibody, or an anti-PD-L1 antibody.

91. A method of decreasing the rate of tumor growth, the method comprising contacting a tumor cell with an effective amount of a composition comprising an antibody or antigen-binding fragment thereof of any one of claims 1-53, the CAR of claim 79, or the antibody-drug conjugate of any one of claims 80-86.

92. A method of killing a tumor cell, the method comprising contacting a tumor cell with an effective amount of a composition comprising the antibody or antigen-binding fragment thereof of any one of claims 1-53, the CAR of claim 79, or the antibody-drug conjugate of any one of claims 80-86.

93. A method of increasing immune response in a subject, the method comprising administering to the subject an effective amount of a composition comprising the antibody or antigen-binding fragment thereof of any one of claims 1-53, the CAR of claim 79, or the antibody-drug conjugate of any one of claims 80-86.

94. A pharmaceutical composition comprising the antibody or antigen-binding fragment thereof of any one of claims 1-53, and a pharmaceutically acceptable carrier.

95. A pharmaceutical composition comprising the antibody drug conjugate of any one of claims 80-86, and a pharmaceutically acceptable carrier.

96. The antibody-drug conjugate of any one of claims 80-86, wherein the drug-to-antibody ratio (DAR) is about 4 or 8.
